(19) United States
(12) Patent Application Publication
(10) Pub. No.: US 2014/0220617 A1
(43) Pub. Date: Aug. 7, 2014

(54) DIALYSIS LIKE THERAPEUTIC (DLT) DEVICE

(71) Applicants: Chong Wing Yung, Milpitas, CA (US); Karel Domansky, Charlestown, MA (US); Richard Terry, Belmont, MA (US); David Kalish, Needham, MA (US); Alexa Schulte, Boston, MA (US); Joo Hun Kang, Boston, MA (US); Donald E. Ingber, Boston, MA (US); Michael Super, Lexington, MA (US); Ryan M. Cooper, Cambridge, MA (US)

(72) Inventors: Chong Wing Yung, Milpitas, CA (US); Karel Domansky, Charlestown, MA (US); Richard Terry, Belmont, MA (US); David Kalish, Needham, MA (US); Alexa Schulte, Boston, MA (US); Joo Hun Kang, Boston, MA (US); Donald E. Ingber, Boston, MA (US); Michael Super, Lexington, MA (US); Ryan M. Cooper, Cambridge, MA (US)

(73) Assignees: CHILDREN'S MEDICAL CENTER CORPORATION, Boston, MA (US); PRESIDENT AND FELLOWS OF HARVARD COLLEGE, Cambridge, MA (US)

(21) Appl. No.: 14/007,738

(22) PCT Filed: Apr. 2, 2012
(86) PCT No.: PCT/US2012/031864

§ 371 Date: Mar. 20, 2014

Related U.S. Application Data
(60) Provisional application No. 61/470,987, filed on Apr. 1, 2011.

(51) Int. Cl. C12Q 1/04; B03C 1/00; B01L 3/00

(52) U.S. Cl. CPC C12Q 1/04; B01L 3/56; B03C 1/002
USPC 435/34; 422/503; 210/695

(57) ABSTRACT

A dialysis like therapeutic (DLT) device is provided. The DLT device includes at least one source channel connected at least one collection channels by one or more transfer channels. Fluid contacting surface of the channels can be an anti-fouling surface such as slippery liquid-infused porous surface (SLIPS). Fluids can be flown at high flow rates through the channels. The target components of the source fluid can be magnetic or bound to magnetic particles using an affinity molecule. A source fluid containing magnetically bound target components can be pumped through the source channel of the microfluidic device. A magnetic field gradient can be applied to the source fluid in the source channel causing the magnetically bound target components to migrate through the transfer channel into the collection channel. The collection channel can include a collection fluid to flush the target components out of the collection channel. The target components can be subsequently analyzed for detection and diagnosis. The source channel and the collection channels of the microfluidic device are analogous to the splenic arterioles and venules, respectively; the transfer channels mimic the vascular sinusoids of the spleen where opsonized particles are retained. Thus, the device acts as a dialysis like therapeutic device by combining fluidics and magnetics.

### CROSS-REFERENCE TO RELATED APPLICATIONS

This application claims benefit under 35 U.S.C. §119(e) of the U.S. Provisional Application No. 61/470,987, filed Apr. 1, 2011, the content of which is incorporated herein by reference in its entirety

### GOVERNMENT SUPPORT

This invention was made with government support under grant no. N66001-11-1-4180 awarded by the Defense Advanced Research Projects Agency (DARPA) and no. W81XWH-07-2-0011 awarded by the Department of Defense. The government has certain rights in the invention.

### TECHNICAL FIELD

The present disclosure relates generally to a microfluidic device with microchannels and methods of use and manufacturing thereof.

### BACKGROUND

Sepsis is a major killer of infected soldiers in the field, as well as patients in state-of-the art hospital intensive care (ICUs), because microbial loads in blood often overcome even the most powerful existing antibiotic therapies, resulting in multi-systems failure and death.

Most DLTs, such as hemofiltration or hemoadsorption systems, use semi-permeable filtration membranes to remove small solutes, and sometimes larger circulating toxins, antibodies and inflammatory mediators that can contribute to multisystem failure in sepsis. However, these methods do not enable most pathogens (e.g., other than some small viruses) to be separated, and removal of anti-microbial immune proteins and cytokines interfere with body's natural protective response to infection. Other technologies being explored for this application use catheters or hollow fibers coated with pathogen-specific ligands (e.g., antibodies, lectins) to pull pathogens out of the blood, but local binding and aggregation of pathogens can disturb blood flow, causing coagulation and clot formation that can be devastating. Ligand-coated surfaces and semi-permeable membranes also can become “fouled” with bound plasma components, serum proteins, or bacterial biofilms. Further, the capacity of these systems is also limited by exposed surface area. Another major limitation is the narrow and specific binding of the ligands, which commonly only recognize one type pathogen or pathogen class.

Accordingly, there is need in the art for an extracorporeal dialysis-like therapeutic (DLT) device that can be inserted into peripheral blood vessels and rapidly clear the blood of infectious pathogens without removing normal blood cells, proteins, fluids or electrolytes can remedy this problem. The present disclosure provides such a dialysis-like therapeutic device.

### SUMMARY

Disclosed herein is a microfluidic device that can facilitate the separation and removal of target components, e.g., pathogens, from a source fluid, e.g., blood, flowing in a source microchannel without removing or altering other components in the source fluid. The fluid can be a liquid or a gas. The target components can be any particulate, molecule or cellular material that is magnetic or can be bound to a magnetic particle introduced to the flowing source fluid.

The source microchannel(s) can be connected to a collection microchannel(s) by one or more transfer channels. The source microchannel(s) and the collection microchannel(s) can be separated by the transfer channel(s) and the source microchannel(s) and the collection channel(s) can be arranged in any orientation, e.g., horizontally co-planar, vertically co-planar, or any angle in between. A collection fluid, flowing in the collection channel(s) can be arranged in used to flush the target components out of the microfluidic device. One or more magnets or a magnetic sources can be positioned adjacent the collection channel(s), or an external magnetic field gradient can be applied, to attract the magnetic target components or magnetic particle bound to the target components into the transfer channels and into the collection channel(s) where they can be carried away in the collection fluid. The magnets or the magnetic field gradient source can be positioned relative to the collection channel(s) to permit the magnetic field gradient to draw the target components or magnetic particle bound to the target components into the transfer channels and the collection channels, but not so strong as to cause the target components or magnetic particle bound to the target components to lodge in the collection channels, unable to be flushed out by the flow of the collection fluid. As one of ordinary skill would appreciate, the position of the magnet or the source of the magnetic field gradient (in the case of an electromagnet) relative to the channels can be determined as a function of any or all of the following: the strength of the magnetic field and field gradient, the magnetic properties of the magnetic particles, the size of the target components and/or the magnetic particles, the size and/or shape of the channels, or the speed and/or viscosity of the fluids used.

The collection fluid containing the target components can be further processed to analyze the target components. The collection fluid containing target components can be collected in a reservoir and batch techniques, such as immunostaining, culturing, polymerase chain reaction (PCR), mass spectrometry and antibiotic sensitivity testing can be used to analyze the target components for use in identification, diagnosis and the like. Alternatively or in addition, the collection fluid containing the target components can be directed into an inline or on-chip diagnostic or analysis device that can process the target components as they flow with the collection fluid. Because target components are either magnet or bound to magnetic particles, magnetic field gradients can be used to collect the target components for inline or on-chip analysis or direct the target components to other devices for detection or analysis.

In operation, the source fluid can be pumped into the source channels and the magnet field gradient can be applied to the source fluid as it flows through the source channel. Pumping can be achieved using a powered or manual pump, centripetal or gravitational forces. The magnetic field can be applied in a direction perpendicular to the direction of fluid flow in order to apply additional forces on the target components carried by the source fluid flowing through the source channel and cause the magnetic target components or the magnetically bound target components to travel into the transfer channels and eventually become drawn into the collection channels. While in some embodiments, the collection channels extended parallel to the source channels, the collection channels can be arranged transverse to the source channels.

In accordance with the invention, the magnet field gradient can apply attraction forces or repulsion forces on the magnetic particles or the magnetic target components to cause them to flow into a transfer channel.

### BRIEF DESCRIPTION OF THE DRAWINGS

The accompanying drawings illustrate exemplary embodiments of the invention and depict the above-mentioned and other features of this invention and the manner of attaining them. In the drawings:

FIG. 1 shows a view of a microfluidic device according to an embodiment of the invention.

FIG. 2 shows a view of a central body of a microfluidic device according to an embodiment of the invention.

FIGS. 3A and 3B show various exemplary branching configurations of microfluidic devices according to the invention.

FIG. 4 shows a cross-sectional view of a microfluidic device according to an embodiment of the invention.

FIGS. 5A-5C show effect of different magnet configurations. FIG. 5A shows a picture of the polysulfone DLT device inserted into the docking station where a single bar magnet is installed. FIG. 5B shows the improved design of the magnetic setup which consists of 6 stationary magnets (assembled together). FIG. 5C, the finite element method magnet (FEMM) revealed that the magnetic flux density gradient was significantly enhanced in a configuration of magnetic setup of FIG. 5B, especially in the middle of the magnet (Δ vs ). This improved configuration of magnetic setup allows ones to utilize extremely enhanced magnetic field gradient (several thousand times larger than that of a single magnet) across the DLT device.

FIG. 6 is photograph showing a central body fabricated from aluminum.

FIG. 7 shows a block diagram of an overall system according to an embodiment.

FIG. 8A shows various views of a syringe mixer.

FIG. 8B is line graph showing binding efficiency of C. albicans using the syringe mixer shown in FIG. 8A.

FIG. 9A shows high magnification view of magnetic antibody opsonins binding specifically to individual C. albicans fungi in whole blood.

FIG. 9B shows lower magnification view of magnetic mannose binding lectin (MBL) opsonin binding multiple fungi pathogens with large magnetic clumps.

FIG. 9C shows lower magnification view of MBL opsonin binding to GFP-labeled E. coli bacteria.

FIG. 9D shows pathogen clearance efficiencies close to 100% at flow rates up to 80 mL/hr can be obtained.

FIGS. 10A and 10B show schematic representations of docking stations.

FIG. 11 shows results of computer simulations of magnetic flux concentrators designed for collection of magnetic beads within a microfluidic device described herein compared with experimental measurements of actual magnetic fields.

FIGS. 12A-12C shows views of a slippery liquid-infused porous surface (SLIPS). An array of micropoasts (1 μm diameter×2 μm space) at low (FIG. 12A) and high (FIG. 12B) magnification, which can create a blood repellent surface by infiltrating spaces with a biocompatible oil that smoothes the rough surface (FIG. 12C).

FIGS. 13A and 13B show fresh unheparinized human blood rapidly clots on conventional glass, PDMS, and Teflon (PTFE) surface, but not on the nanostructured Teflon surfaces impregnated with biocompatible oil (Oil-Infiltrated PTFE).

FIG. 14A shows an experimental setup for circulating blood through the dialysis like therapeutic (DLT) system using a peristaltic pump. Blood flows from the Vacutainer tubes to the polysulfone DLT device through the peristaltic pump.

FIGS. 14B and 14C show that after running heparinized human whole through the device at 100 and 200 mL/h for 2 hours, the devices were washed by flowing PBS buffer for 5 min and no blood clots were found at both flow rates (FIG. 14B, 100 mL/h) and (FIG. 14C, 200 mL/h) for 2 hours.

FIG. 14D shows that circulation of non-heparinized human blood formed large blood clots and clumps in the channels when blood was flown at 100 mL/h for 2 hours.

FIGS. 15A and 15B show that two DLT devices connected in parallel can dramatically increase throughputs up to 836 mL/h of blood. Two DLT devices were inserted in the top and the bottom slots of the docking station and blood collected from two outlets was analyzed to determine isolation efficiency of the spiked C. albicans into blood.

FIG. 16A is a line graph and a bar graph showing improvements in device design and pathogen separation. Candida albicans pathogens were pre-bound to MBL-coupled 1 micron beads and spiked into heparin anticoagulated human blood. Line graph shows data with a S-layer polysulfone device based on the previous design and MBL-coupled 1 micron beads presented in QPR1. Bar graph shows data with MBL-fp1 (FcMBL: IgG Fc fused to mannose binding lectin) coated magnetic beads and the new laminated device/multiple magnet setup. With the new design, >99% of the pathogens were removed at flow rates of 360 mL/hr whereas, with the previous design, the isolation efficiency fell to 36% at 360 mL/hr.

FIG. 16B shows improvements in device design and pathogen separation. Photograph, an exemplary setup of the laminated DLT device with multiple magnets. Line graph, Candida albicans pathogens were pre-bound to MBL-coupled 1 micron beads and spiked into heparin anticoagulated human blood. Data from a 3 layer device based on the previous design were compared with the two cassettes of the new laminated device running in parallel. With the new design, >85% of the pathogens were removed at flow rates of 836 mL/hr whereas, with the previous design, the isolation efficiency fell to 36% at 360 mL/hr.

FIG. 17A is a schematic representation of a DLT system integrated with an in-line mixer and a syringe pump for adding magnetic beads into blood in tubing continuously. The blood sample mixed with the magnetic beads added throughout the in-line mixer flows into the DLT device and then magnetically labeled pathogens are removed from blood, and then cleansed blood flows out through the outlet that can be connected to a femoral catheter on the rat sepsis model.

FIG. 17B shows a “simplified animal” model for using the microfluidic device for pathogen clearance/separation from blood. A disposable in-line mixer (OMEGA Engineering Inc.,) was used to introduce MBLfp1 beads into blood containing spiked C. albicans. In this simplified animal model, 88% of Candida were cleared from the blood at a flow rate of 10 mL/hr through the DLT Device.

FIG. 18 is a photograph of a bubble trapping device. This device removes all bubbles coming in through the tubing by buoyancy of air bubbles that move upward rapidly, and liquid solution without bubbles flows through the device. An excess amount of large air bubbles can be removed from the 3-way valve.

FIG. 19 shows schematic representation of a microfluidic device fabricated from four polysulfone plastic layers. The device comprises a source channel positioned between two collection channels.

FIG. 20 shows a schematic representation of multiplexing multiple microfluidic devices in parallel to create a biomimetic spleen device with high throughput (>1.25 L/hr) flow capabilities.

### DESCRIPTION OF EXEMPLARY EMBODIMENTS

Disclosed herein is a fluidic device that can facilitate the separation and removal of target components from a source fluid flowing in a source channel without removing or altering other components in the source fluid.

The fluid can be a liquid or a gas. The target components can be any particulate, molecule or cellular material that is magnetic or can be bound to a magnetic particle introduced to the flowing fluid. Multiple fluidic devices can be coupled together in series and/or parallel to improve the throughput and efficiency of the system. The target components are collected in a collection fluid that can be further processed to analyze the target components. The collection fluid containing target components can be collected in a reservoir and batch techniques, such as immunostaining, immunoassaying, culturing, polymerase chain reaction (PCR), mass spectrometry, and antibiotic sensitivity testing can be used to analyze the target components for use in identification, diagnosis, and the like. Alternatively, the collection fluid containing the target components can be directed into an inline or on-chip diagnostic or analysis device that can process the target components as they flow with the collection fluid. Because target components are either magnet or bound to magnetic particles, magnetic field gradients can be used to collect the target components for inline or on-chip analysis or direct the target components to other devices for detection or analysis.

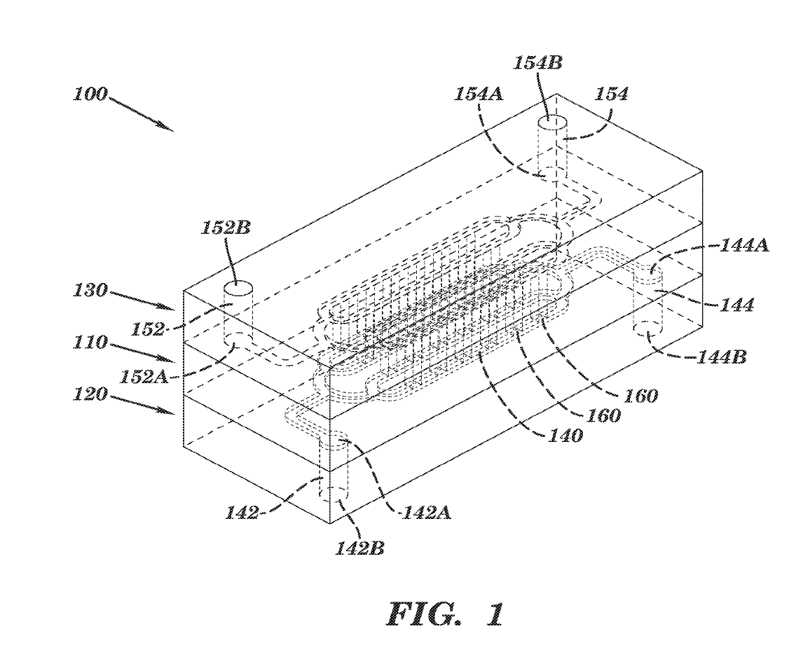

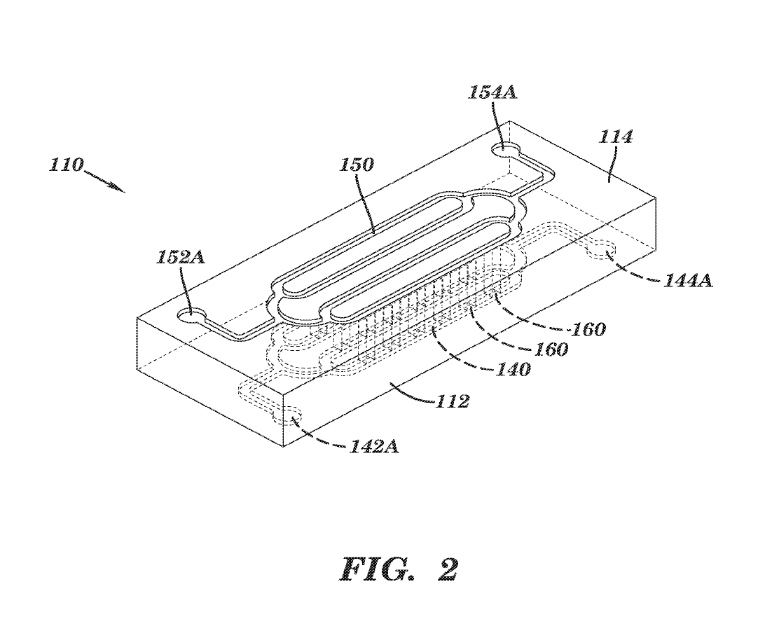

FIG. 1 shows the microfluidic device 100 in accordance with an embodiment of the present disclosure. The microfluidic device 100 shown in FIG. 1 can include a rectangular body although other shapes can also be used (e.g. circular, elliptical, trapezoidal, polygonal, and the like). As shown in FIG. 1, the microfluidic device can include a central body 110, shown in more detail in FIG. 2, and outer laminating layers 120 and 130. The central body 110 comprises a first outer surface 112 which is in contact with laminating layer 120 and a second outer surface 114 which is contact with laminating layer 130. Surfaces 112 and 114 can be the opposing surfaces of the central body 110. The laminating layers 120 and 130 can be bonded to the surface of the central body by medical grade adhesive.

As shown in FIG. 2, surface 112 of central body 110 can include one or more source fluid channels 140 extending between one or more inlets 142A and one more outlets 144A. As shown in FIG. 1, the one more inlets 142A can be in communication with inlet ports 142 extended from an aperture 142B on the outer surface 122 of the laminating layer 120. The one more inlets 144A can be in communication with inlet ports 144 extended from an aperture 144B on the outer surface 122 of the laminating layer 120. Inlet port 142 and outlet port 144, while shown oriented perpendicular (i.e., along the z-direction) to the source fluid channels 140, can be oriented in any angel (including straight through) with respect to the source fluid channels 140. The source fluid containing the target components flows into the source channels 140 through one or more inlet ports 142 and exits from the microfluidic device 100 through one or more outlet ports 144.

While the collection channels 150 are shown extending parallel to the source channels 140, in some embodiments, the collection channels 150 can extend perpendicular to (or angle) to the source channels 140. They can be arranged horizontally or vertically.

The source fluid channels 140 can extend along the length of the central body 110 (e.g. the y-direction), as shown in FIG. 2. The source channels 140 can be of any polygonal, non-polygonal, circular, or oval cross-section. In some embodiments, the source channel 140 can be rectangular in cross-section. The cross-sectional dimension of the individual source fluid channels 140 can be designed to more effectively expose the target components to the magnetic field and guide the attracted target components toward the transfer channels 160. In one embodiment, the source fluid channels 140 can have a flattened geometry in order to maximize the area of exposure to the magnetic fields. In addition, the source fluid channels 140 can be designed to slow the flow rate of the source fluid as it passes through the source channels 140 to maximize the number of magnetically bound target components to migrate into the transfer channels 160.

As shown in FIG. 2, surface 114 of central body 110 can include one or more collection fluid channels 150 extending between one or more inlets 152A and one more outlets 154A. As shown in FIG. 1, the one more inlets 152A can be in communication with inlet ports 152 extended from an aperture 152B on the outer surface 132 of the laminating layer 130. The one more inlets 154A can be in communication with inlet ports 154 extended from an aperture 154B on the outer surface 132 of the laminating layer 130. Inlet port 152 and outlet port 154, while shown oriented perpendicular (i.e., along the z-direction) to the collection fluid channels 150, can be oriented in any angel (including straight through) with respect to the source fluid channels 150. The collection fluid flows into the collection channels 130 through one or more inlet ports 132 and exits from the microfluidic device 100 through one or more outlet ports 134.

Like the source channels 140, the collection channels 150 can be of any polygonal, non-polygonal, circular, or oval cross-section. However, it is to be understood that cross-section of each source channel 140 and collection channel 150 is independently selected. Thus, the cross-section of all of the source channels 140 and collection channels 150 can be the same, all different, or any combinations of same and different. In some embodiments, collections channels 140 can be rectangular in cross-section.

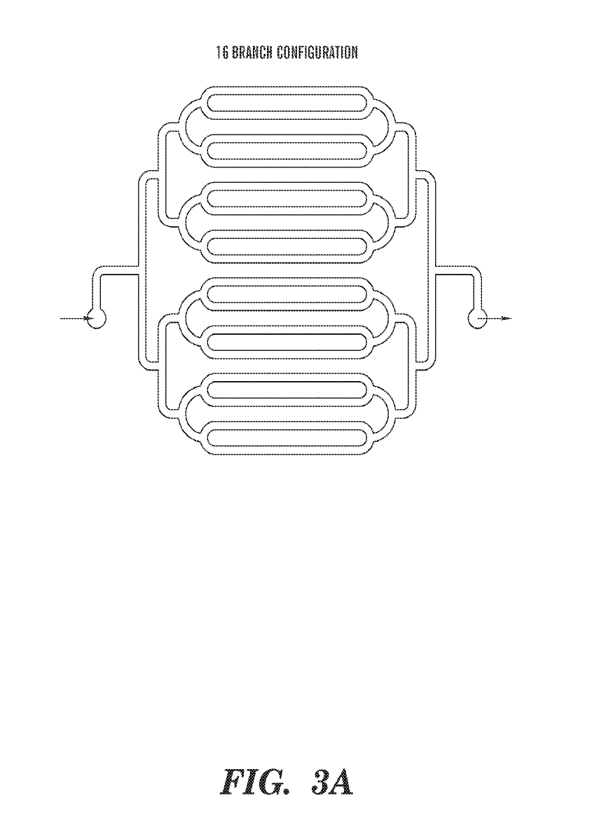

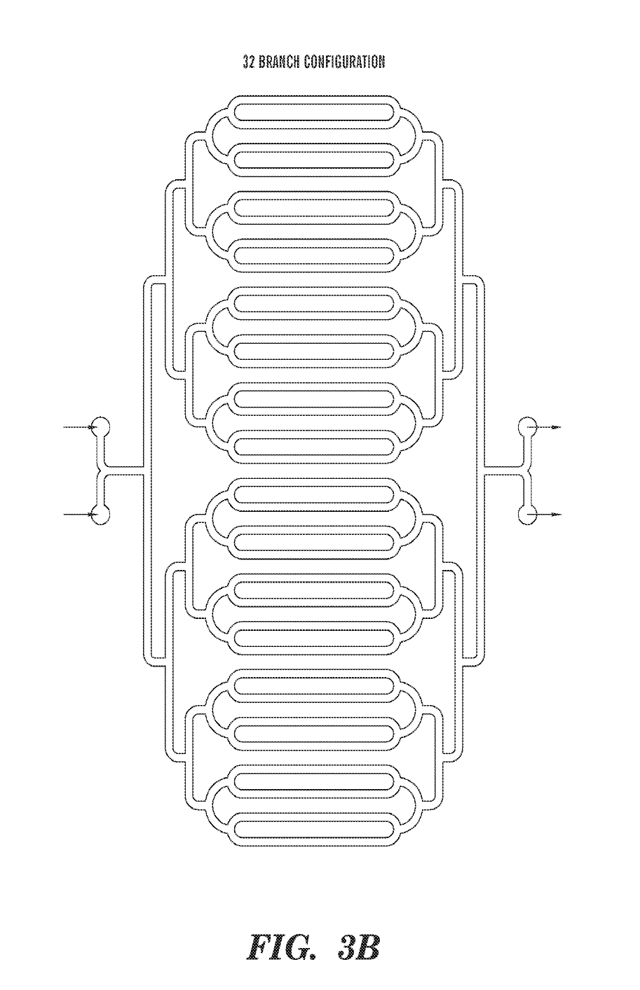

As shown in FIG. 2, the central body 110 can include one or more transfer channels 160 connecting the source channels 140 with the collection channels 150. While the transfer channels 160 are shown oriented substantially perpendicular to the source channels 140 and collection channels 150, the transfer channels 160 can be oriented in a range of angles (e.g., 1 to 90 degrees, where 0 degrees corresponds to the direction of flow in the source channels 140, see FIG. 3) with respect to the source channels 140. In some embodiments, the transfer channels 160 can be oriented substantially perpendicular to the collection channel 150 and the source channel 140. This perpendicular configuration can exploit the Bernoulli principle that the collection fluid flowing in the collection channel 150 will have the lower static pressure compared to the fluid in the transfer channel(s) 160 and cause the magnetic beads and bound target components in the transfer channel(s) 160 to be drawn into the collection fluid.

The transfer channels 160 can be of any polygonal, non-polygonal, circular, or oval cross-section. In some embodiments, the transfer channels can be rectangular in cross-section. The transfer channels 160 serve to transport target components, e.g., magnetic particle bound target components, from the source channels 140 to eventually be flushed out of the microfluidic device 100 via the collection channels 150. The target components bound to the magnetic particles can be separated from the remaining components of the source fluid flowing in the source channels 140 by applying an external magnetic force that drives the magnetic particles into the transport channels 160. While the transfer channels 160 are shown having 90 degree corners, other corner angles and shapes, such as angles higher or lower than 90 degrees or rounded corners, can also be utilized. The spacing between transfer channels can also be adjusted as desired. For example, the transfer channels can be spaced apart by about 10 μm to about 5 mm. In some embodiments, the transfer channels can be spaced apart by about 100 μm to about 500 μm.

The number, size, shape, orientation and spacing of the source fluid channels 140 and the collection fluid channels 150, as well as the transfer channels 160 can be varied depending on the desired system performance and efficiency.

The source fluid channels 140 and the collection fluid channels 150 can independently have a length of about 1 mm to about 10 cm, a width of about 0.1 mm to about 10 mm and a depth of about 0.1 mm to about 2 mm. In some embodiments, the source channels 140 and the collection channels 150 have the same dimension, i.e., same length, width, and depth.

In one preferred embodiment, the source channel 140 for transporting source fluid can be 2 cm long by 2 mm wide by 0.16 mm high.

In some embodiments, the collection channels 150 for transporting collection fluid can be independently 2 cm long by 2 mm wide by 0.16 mm high.

In some embodiments, the transfer channels 160 have a cross-section dimension of about 1 mm×200 μm to about 10 mm×1 mm. In some embodiments, the transfer channels 160 have a cross-section dimension of about 100 um (thickness)×100 um (width) to about 1 mm×400 um.

As shown in FIG. 1 the outer surfaces 112 and 114 of the central body 110 can be laminated with laminating layers 120 and 130 respectively to form a sealed and enclosed set of channels which allows the fluids to travel between the device without leakage or such. Surface of the laminating layer 120, which is in contact with the central body 110 can include a portion of the source fluid channels 140, inlets 142A, or outlets 144A, i.e., a part of the source fluid channels 140, inlets 142A, or outlets 144A is in the laminating layer 120. Alternatively, the laminating layer 112 does not include a portion of the source fluid channels 140, inlets 142A, or outlets 144A, i.e., the source fluid channels 140, inlets 142A, or outlets 144A are fully in the central body.

Similarly, surface of the laminating layer 130, which is in contact with the central body 110 can include a portion of the collection fluid channels 150, inlets 152A, or outlets 154A, i.e., a part of the collection fluid channels 150, inlets 152A, or outlets 154A is in the laminating layer 130. Alternatively, the laminating layer 130 does not include a portion of the source fluid channels 150, inlets 152A, or outlets 154A, i.e., the source fluid channels 150, inlets 152A, or outlets 154A are fully in the central body.

It should also be noted that the configurations of one or more of the microchannel assemblies as well as the overall device can have other designs and should not be limited to that shown in the figures. Further, although the channels in the channel assemblies may be shown to have a circular cross section, the channels can have other cross-sectional shapes including, but not limited to square, rectangular, oval, polygonal and the like, or channels that vary in their dimensions and shape along their length as can be created with micromaching technologies.

As shown in FIGS. 1 and 2, the source fluid channels 140 as well as the collection fluid channels 150 can branch out into individual branches from their respective inlet ports and the individual branches of the source fluid channels 140 and the collection channels 150 converge to their respective outlet ports. Although four branches are shown in FIGS. 1 and 2 any number of branches, even one branch, can be used. For example, FIG. 3A illustrates 16 branches each of the collection channels and source channels, and FIG. 3B illustrates 32 branches each of collection channels and source channels in accordance with the invention. As one of ordinary skill will appreciate, the number of branches can be selected as a function of the desired performance and efficiency of the system.

The source fluid channels 140 and the collection fluid channels 150 can mirror each other and have the same or similar branched configuration. In addition, each individual branch of the source channel 140 and the corresponding branch of the collection channels 150 can include at least one transfer channel 160 connecting them.

The source channels 140 and the collection channels 150 can be substantially parallel to each other. The spacing between the source channel 140 and the collection channel 150 can range from about 5 μm to about 10 mm. In some embodiments, the spacing between source channels 140 and the collection channels 150 can range from about 10 um to 500 um.

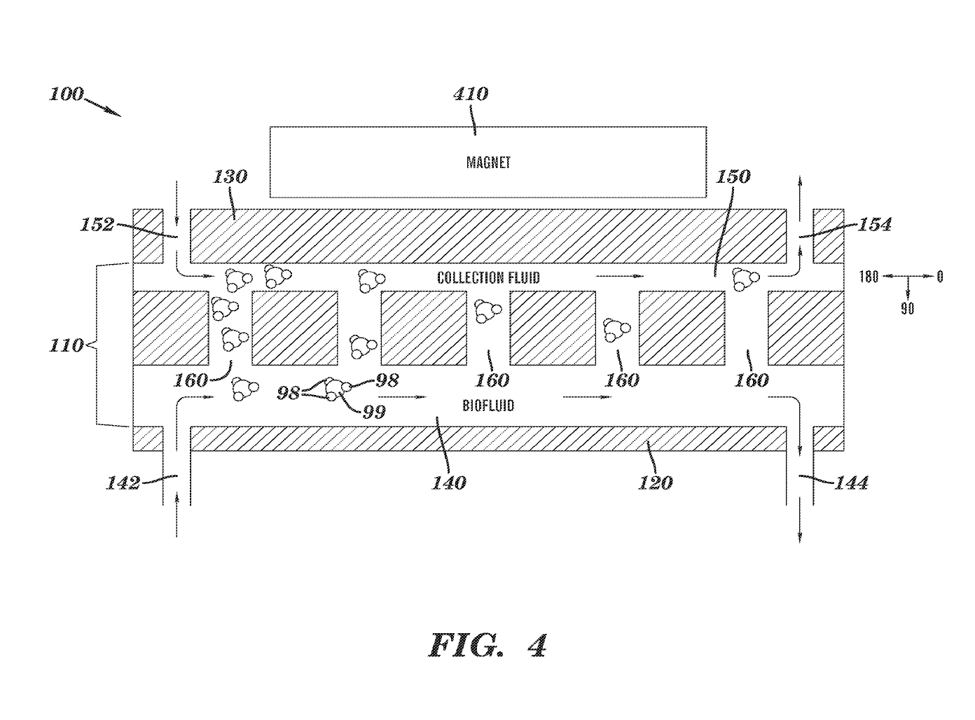

FIG. 4 illustrates a cross-sectional view of a microfluidic device in accordance with the present invention. As shown in FIG. 4, a source fluid enters the source channel 140 via the inlet port 142, wherein the source fluid (shown by arrows) passes through the device 100 via the source channel 140 and exits the device 100 via outlet port 144.

The source fluid can be a source fluid that contains target components 99, such as pathogens, including bacteria and yeast, cancer/tumor cells or a desirable target component such a stem cell, fetal cell, cytokine or antibody. These target components 99 can be mixed with magnetic particles 98 which are conditioned or modified to attach to the predetermined target components 99 prior to entering the microfluidic device 100.

In order to capture the target components 99 from the flowing source fluid, one or more magnetic sources 410, such as Neodymium magnets, can be positioned adjacent to the collection channels 150 of the microfluidic device 100. It should be noted that other types of magnets can be used and are thus not limited to Neodymium. For instance the magnet(s) can be made of Samarium Cobalt, Ferrite, Alnico and the like, or an internal or external electromagnet may be used to generate magnetic field gradients. As shown in FIG. 4, the magnet 410 is positioned vertically over the transfer channels 160, such that magnetic field gradient applied by the magnet 310 attracts the magnetic beads 98 and cause the magnetic beads 98 to move toward the magnet 310. Specifically, the magnetic field gradient from the magnet 410 causes the magnetically bound target components 99 in the source fluid to migrate through the transfer channels 160 and into the collection channels 150. These components can be removed and collected when the collection fluid is flushed there through. In some embodiments of the invention, the magnetically bound target components 99 can migrate into and settle in the transfer channels 160 to be drawn into the collection channel 150 by the flushing operation. It should be noted that although the source fluid and the collection fluid are shown flowing in the same direction within the microfluidic device 100, the source fluid and the collection fluid can flow in opposite directions within the microfluidic device 100.

As shown in FIG. 4, collection fluid enters the collection fluid channel 150 via inlet port 152 and passes through the collection fluid channel 110 toward the outlet port 154. The inlet ports 106A and 106B can be the same inlet port and outlet ports 108A and 108B can be the same outlet port.

It should be noted that the collection channels 150, and desirably the ports 152 and 154, are filled to capacity with the collection fluid. However, in some embodiments, the collection fluid does not continually flowing through the collection channel 150, and instead is flowed through the collection channel 150 intermittently or on a periodic basis where there are intervals in which the collection fluid flows and intervals in which the collection fluid is stationary or flows at a slower rate. Because the collection fluid is not continuously flowing, but is allowed to become stagnant in the collection channel 150, the magnetically bound target components entering the transfer channels can become retained in these transfer channels 160 for a time without exiting the device.

Once the collection fluid begins flowing, changing from the stagnant condition to a flowing condition in the collection channel 150, the magnetically bound target components remaining in the transfer channels 160 can be drawn into the collection channel 150, analogous to the periodic flow of lymph fluid that carries away waste material from the sinuses of the spleen. The flowing collection fluid in the collection channels can have a lower static pressure relative to the transfer channels and cause the magnetic beads and bound target components present in the transfer channels to flow into the collection fluid stream. This predetermined pressure or flow differential can be created when the collection fluid flows through the collection channels 150 during the “flushing” operation, wherein the flushing operation can be controlled to have a desired duration. By controlling the duration of the flushing operation, the amount of source fluid that transfers into the collection channels 150 can also be controlled.

The microfluidic devices can include one or more optical or impedance microelectronic sensors integrated therein which detect target component or pathogen buildup. The microfluidic devices can incorporate a feedback loop in which sensors communicate with a controller and/or one or more pumps to automatically control the flow (e.g. start/stop duration, flow rate, and the like) of the collection fluid. In addition, one or more magnetic bead traps, external to the microfluidic device, can be used in the system in FIG. 1 to remove any remaining particles that are not cleared by other mechanisms before the source fluid is returned to the source or input to the source fluid collector. The microfluidic device can include one or more valves at the inlets and/or outlets of the collection channels and/or source fluid channels. The microfluidic device can include one or more valves at the transfer channels to control the flow of the magnetically bound target components entering or exiting the transfer channels.

To provide high throughput, two or more of the microfluidic devices can be multiplexed together in a multiplexed system. For example, one, two, three, four, five, six, seven, eight, nine, ten, eleven, twelve, thirteen, fourteen, fifteen or more microfluidic devices can be connected together. In the multiplexed system, the microfluidic devices can be connected together in series or parallel to maximize the cleansing efficiency or throughput flow rate, respectively.

For parallel connection. The source inlet of each device can be connected to the same source fluid source and the source outlet can be connected to the same source fluid collector. For connection in series, source outlet of one microfluidic device can be connected to the source inlet of a second device. In addition, the microfluidic devices in a multiplexed system can be placed such that two microfluidic devices can share a magnetic source.

In a multiplex system, multiple microfluidic devices can be connected together using spacers. Spacers can be fabricated from the same material as the microfluidic devices. The spacers can provide gaps between the individual microfluidic devices for insertion of magnets and can contain holes for interconnecting source channel and collection channel ports of individual microfluidic devices. When the source fluid is a biological fluid, e.g., blood, the end microfluidic device of the multiplexed system can contain a bonded block with standard blood and saline connectors. The multiplexed devices can be cleaned, sterilized, and inserted into sterile bags to be opened immediately prior to use. The channel geometry, number of channels per device, and number of devices per multiplexed system can be optimized to satisfy the desired source fluid, e.g., blood, flow capacity as well as pathogen separation efficiency.

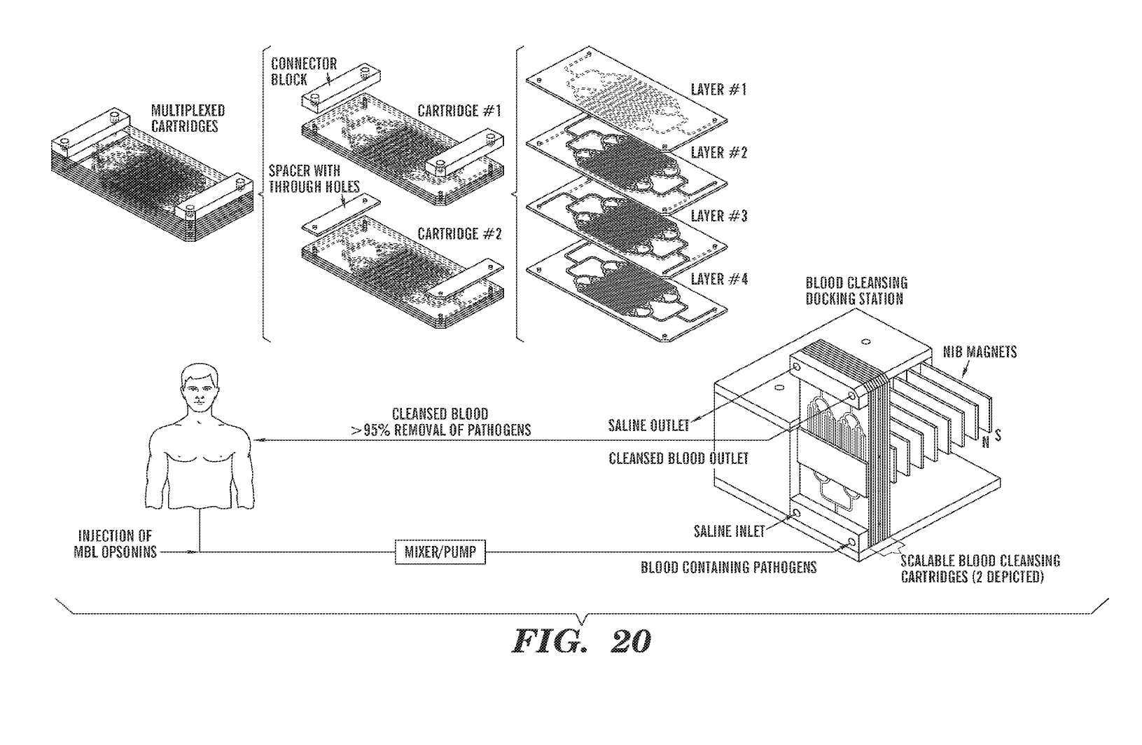

When the source fluid is blood, the source channel and the collection channels of the microfluidic device are analogous to the splenic arterioles and venules, respectively; the transfer channels mimic the vascular sinusoids of the spleen where flow is episodic and opsonized particles are retained; and the carrier fluid channels mimic the lymphatic fluids that eventually clear the opsonized particles. FIG. 20 shows a schematic representation of multiplexing multiple microfluidic devices in parallel to create a biomimetic spleen device with high throughput (>1.25 L/hr) flow capabilities.

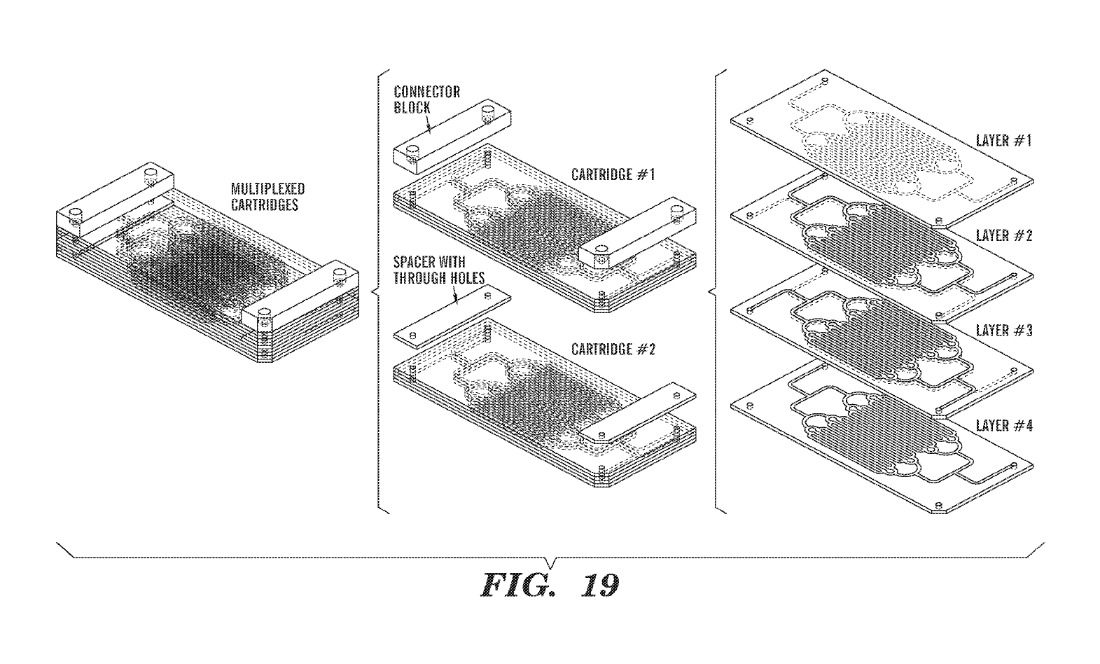

To further increase the throughput of the microfluidic device, the microfluidic device can be comprises a source fluid channel positioned between two collection channels. The source fluid channel can be connected to each of the two collection channels by one or more transfer channels. For example, over 95% of all bead-bound fungal pathogens was separated from whole blood with flow rates of up to 80 mL/hr using a 16-channel PDMS microfluidic device with channel cross-sections of 2×0.16 mm from a single source fluid channel aligned with a single collection fluid channel. By doubling the cross-section to 2×0.32 mm and using two collection channels (one above and one below the source channel), similar clearance efficiencies can be obtained at maximum flow rates ˜1600 mL/hr. FIG. 19 shows how a microfluidic device can be constructed from four polysulfone plastic layers comprising a source channel positioned between two collection channels. Fluids such as blood and saline flow in “fluidic-layer” that are formed between the “plastic-layers” which have recessed channels features micromilled on their surfaces. The blood-fluidic-channel, i.e., the source channel, is formed between plastic-layers 2 and 3. Plastic layers 1 and 2, as well as 3 and 4 form saline-fluidic-layers, i.e., collection channels, above and below the blood-fluidic layer.

To minimize the risk that the platelets activate and induce clotting, the shape of the channels can be carefully chosen to mimic the shape of living, high flow blood vessels (e.g., aorta of small animals) and hence to minimize shear. Channel geometry and flow rate can be optimized to minimize shear disturbances throughout the channel via computer simulations (Fluent and CFX software packages of ANSYS) of non-Newtonina fluid dynamics. Multiphase simulations between blood and saline can be used to minimize mixing and blood loss or dilution. If unmodified machined surfaces induce blood clotting in the presence of heparin, they can be physically or chemically modified (chemical vapor polishing, plasma treatment, nanpatterning, etc.) to provide an anti-fouling surface.

Other channel considerations include the rapidly decaying reach of the magnetic field which can limit the channel depth, the diminishing structural integrity of the channels with increasing width, and the increasing shear stress with decreasing channel dimensions.

Blood clotting on synthetic surfaces is a long-standing and widespread problem in medicine, which is initiated on surfaces by protein absorption that promotes platelet adhesion and activation, as well as release of thrombin that activates fibrin clot formation. Accordingly, the fluid contacting surfaces of the microfluidic device, e.g., channels or tubing or catheters that connect the device to a source or collector, can be coated or treated to resist degradation or facilitate flow and operation. For example, fluid contacting surface of the source fluid channels, the collection channels, the transfer channels, or the tubing or catheter connecting the channels to fluid sources can be an anti-fouling surface.

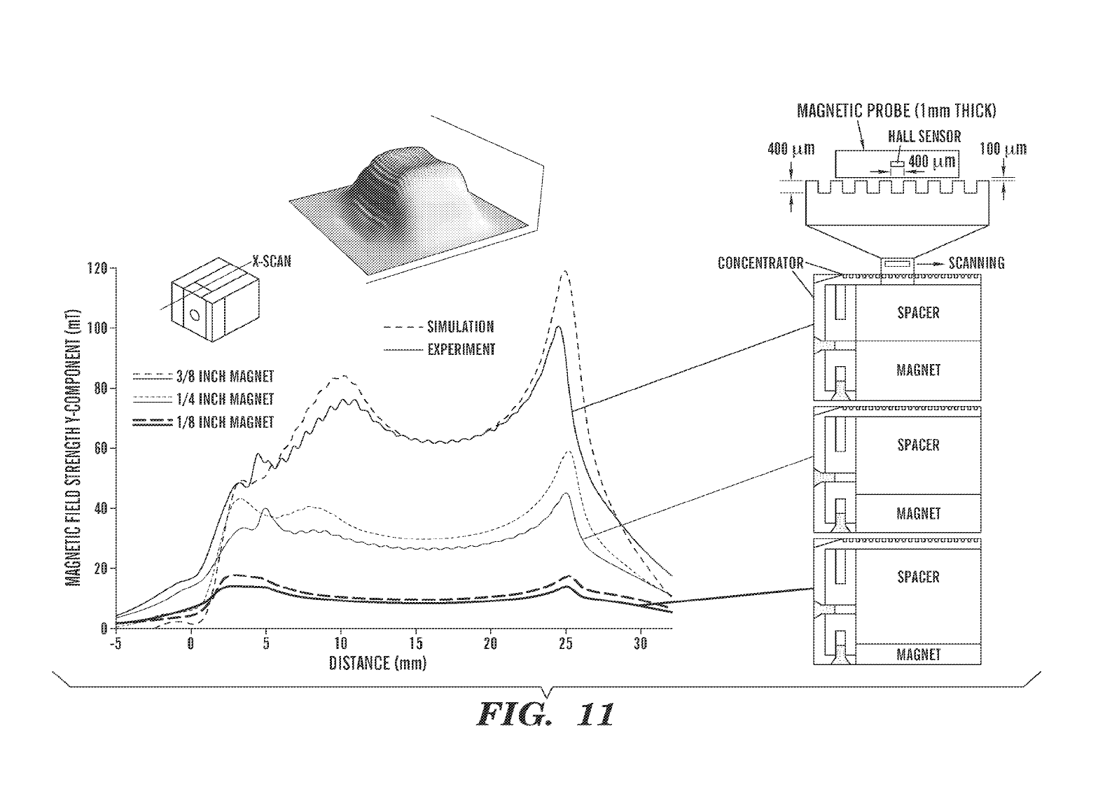

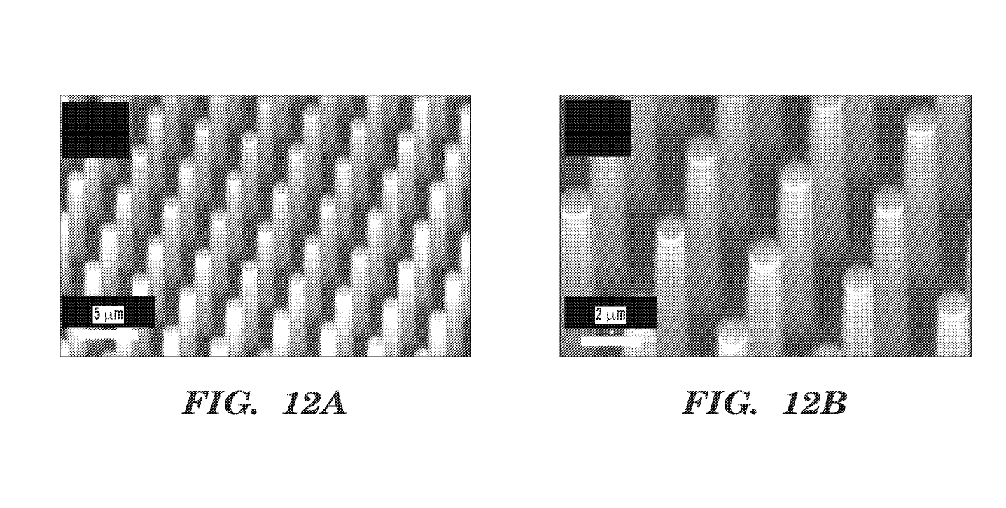

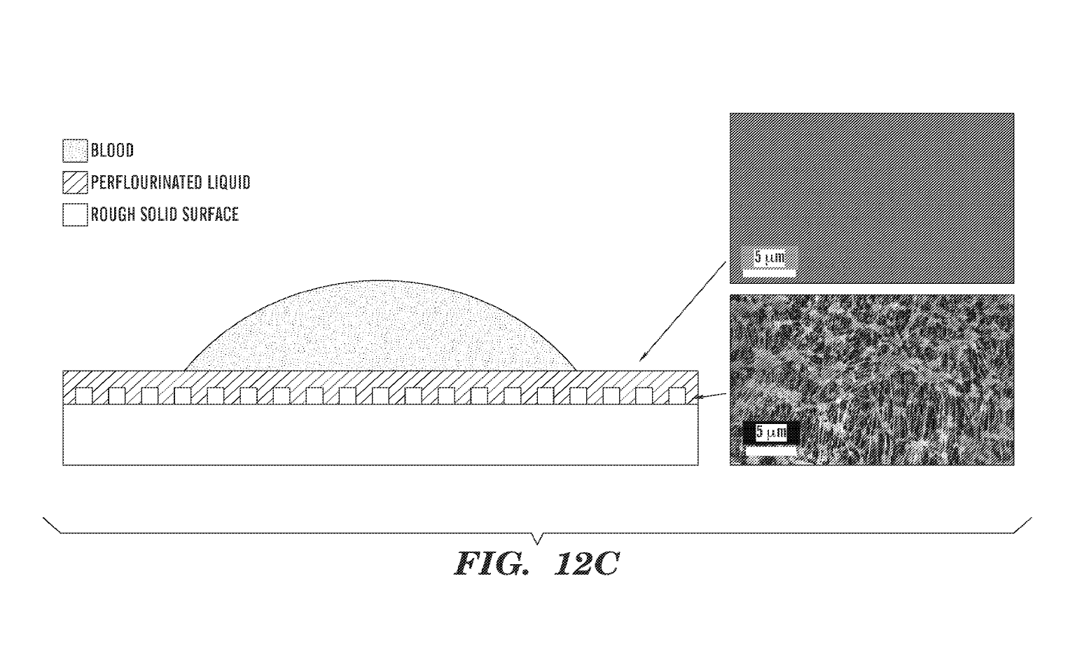

Wong et al., Nature, 2011, 477: 443-447, content of which is incorporated herein by reference, describe anti-fouling surfaces that can be employed for a microfluidic device described herein. As described in Wong et al. an anti-fouling surface can employ an array of nano- and micro-structures separated by infiltrating layer of low surface energy, chemically inert, perfluorinated oil, which is held in place by features of the surface structures (FIG. 12).

The combination of these can produce a physically smooth lubricating film on the surface because the porous structure holds the low energy liquid in place. This thin lubricating film minimizes surface inhomogeneities, reduces retention forces and enhances liquid mobility along the surface, not unlike the lipid bilayer of cells. Hence, contact with the surface is minimal, and the liquid remains highly mobile. The lubricating film can be generated by a liquid imbibing process induced by porous materials as described, for example in, Wenzel, R. N. Ind. Eng. Chem. 1936, 28: 988-994 and Courbin, L., et al. Nature Materials, 2007, 6: 661-664. The physical roughness of the porous material not only induces wetting of the lubricating fluid, it also can provide additional surface area for adhesion of the lubricating fluid to the surface.

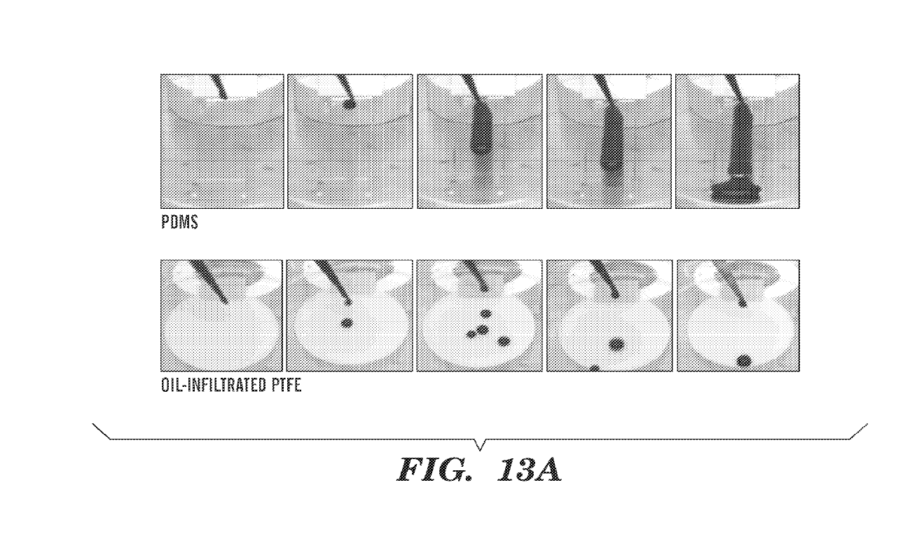

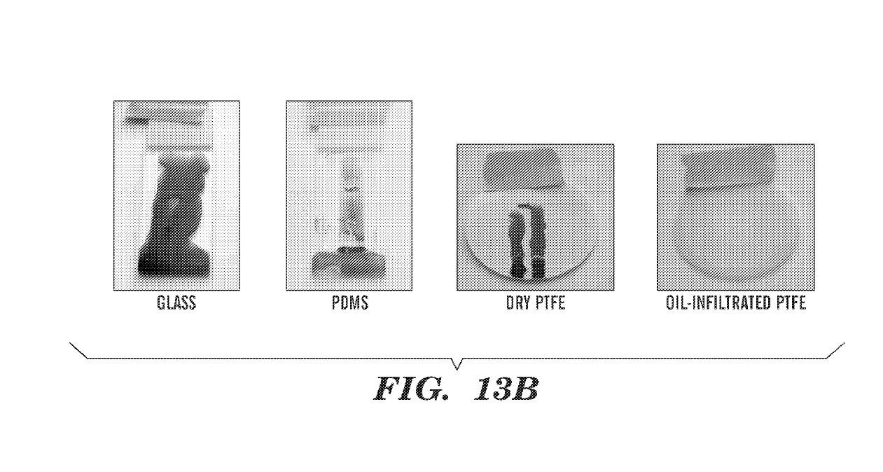

The “liquid-like” surface can be extremely effective at preventing adhesion of platelets and fibrin clot formation when in contact with fresh unheparinized human blood. As seen in FIG. 13A, fresh, whole, human, unhepranized blood (0.75 mL) beaded up and slid off substrates composed of microstructured PTFE (Teflon; 1 μm pore size) impregnated with perfluorinated oil (Flluorinert FC-70, 3M Corp.), whereas it rapidly coagulated and adhered to control smooth PTFE, as well as glass.

Thus, this property represents a first of its kind since no other artificial surface is able to prevent the activation and thrombosis for extended periods of time. These anti-coagulant surfaces offer a new way to control adhesion of blood components and clot formation. In addition, these anti-coagulant surfaces can support blood flow through the microfluidic device without producing coagulation. Hence the need for adding anti-coagulant agents into the blood or in the microfluidic device can be reduced. The “liquid-like” surface is also referred to as a slippery liquid-infused porous surface (SLIPS).

Micromolding techniques can be utilized to create arrays of hydrophobic raised surface structures at the micrometer scale, such as posts and intersecting walls patterned in polymers, such as Teflon or polysulfone, which is already FDA approved for blood compatibility. The infiltrating liquid can be selected from a number of different liquids, such as FDA-approved polyfluoroalkoxy (PFA). The fabricated anti-coagulant surface is smooth and it is capable of repelling a variety of liquids, including blood. A range of surface structures having different feature sizes and porosities can be utilized, to determine their effectiveness for confining the infiltrating liquid or for resisting attachment of blood components and clots. Arrays of nanostructured posts in silicon substrates can be fabricated to leverage the precision of semiconductor processing methods and techniques. The post array substrate can be used as masters for making replica in FDA-approved materials, such as polysulfone or PDMS. Feature sizes can be in the range of hundreds of nanometers to microns (e.g., 100 to 1000 nm), and with aspect ratios from about 1:1 to about 10:1. Porous nano-fibrous structures can be generated in situ on the fluid contacting surface of metallic microfluidic devices using electrochemical deposition. In situ synthesis of biocompatible polypyrrol nanostructures in diversity of morphologies and porosities is known in the art. See for example, U.S. Prov. Pat. App. No. 61/353,505, filed Jul. 19, 2010 and Kim, P. et al., Nano Letters, in press (2011).

These structures can be utilized to determine the optimal wetting and adhesion of different lubricating liquids. A number of different oils can be utilized from the family of polyfluorinated compounds. The candidates can be selected on the basis of their anti-clotting performance, chemical stability under physiological conditions, and levels of leaching from the surface of the devices. For example, compounds that are approved for use in biomedical application (e.g. blood substitutes, MRI contrast agents, and the like), can be utilized. In some embodiments, PFC Perflubron or Perfluorooctylbromide (Alliance Pharmaceutical) can be utilized.

The surfaces can be analyzed after exposure to blood to look for evidence of platelet or fibrin adhesion using surface characterization techniques, such as fluorescence and scanning electron microscopy (SEM). Polyflurinated compounds have poor solubility in a variety of solvents, which can raise certain challenges for monitoring. In order to overcome these challenges, the analysis can involve a combination of extraction into a fluorinated solvent, followed by chromatography, mass spectrometry, and ¹⁹F—NNMR.

After testing the effectiveness and stability of these surfaces in the presence of high blood flows, the structural design (i.e., post-spacing, pore size, and the like) can be further optimized to minimize any effects of fluid leeching. A range of accelerated leaching tests at higher than body temperatures can be performed, in order to acquire data that can be translated to the long-term performance of the non-fouling surface in contact with biological fluids. While many of these compounds are reported to be non-toxic, necessary toxicological screening of the selected impregnating fluids can be performed when desired.

In some embodiments, fluid contacting surfaces of the microfluidic device, e.g., channels, tubing or catheters, can be coated by an anti-coagulant agent. Exemplary anti-coagulants include, but are not limited to, heparin, heparin substitutes, salicylic acid, D-phenylalanyl-L-prolyl-L-arginine chloromethyl ketone (PPACK), Hirudin, ANCROD® (snake venom, VIPRONAX®), tissue plasminogen activator (tPA), urokinase, streptokinase, plasmin, prothrombopenic anticoagulants, platelet phosphodiesterase inhibitors, dextrans, thrombin antagonists/inhibitors, ethylene diamine tetraacetic acid (EDTA), acid citrate dextrose (ACD), sodium citrate, citrate phosphate dextrose (CPD), sodium fluoride, sodium oxalate, potassium oxalate, lithium oxalate, sodium iodoacetate, lithium iodoacetate and mixtures thereof.

Suitable heparinic anticoagulants include heparins or active fragments and fractions thereof from natural, synthetic, or biosynthetic sources. Examples of heparin and heparin substitutes include, but are not limited to, heparin calcium, such as calciparin; heparin low-molecular weight, such as enoxaparin and lovenox; heparin sodium, such as heparin, lipo-hepin, liquaemin sodium, and panheprin; heparin sodium dihydroergotamine mesylate; lithium heparin; and ammonium heparin.

Suitable prothrombopenic anticoagulants include, but are not limited to, anisindione, dicumarol, warfarin sodium, and the like.

Examples of phosphodiesterase inhibitors suitable for use in the methods described herein include, but are not limited to, anagrelide, dipyridamole, pentoxifyllin, and theophylline.

Suitable dextrans include, but are not limited to, dextran70, such as HYSKON™ (CooperSurgical, Inc., Shelton, Conn., U.S.A.) and MACRODEX™ (Pharmalink, Inc., Upplands Vasby, Sweden), and dextran 75, such as GENTRAN™ 75 (Baxter Healthcare Corporation).

Suitable thrombin antagonists include, but are not limited to, hirudin, bivalirudin, lepirudin, desirudin, argatroban, melagatran, ximelagatran and dabigatran.

As used herein, anticoagulants can also include factor Xa inhibitors, factor Ha inhibitors, and mixtures thereof. Various direct factor Xa inhibitors are known in the art including, those described in Hirsh and Weitz, Lancet, 93:203-241, (1999); Nagahara et al. Drugs of the Future, 20: 564-566, (1995); Pinto et al, 44: 566-578, (2001); Pruitt et al, Biorg. Med. Chem. Lett., 10: 685-689, (1000); Quan et al, J. Med. Chem. 42: 2752-2759, (1999); Sato et al, Eur. J. Pharmacol, 347: 231-236, (1998); Wong et al, J. Pharmacol. Exp. Therapy, 292:351-357, (1000). Exemplary factor Xa inhibitors include, but are not limited to, DX-9065a, RPR-120844, BX-807834 and SEL series Xa inhibitors. DX-9065a is a synthetic, non-peptide, propanoic acid derivative, 571 D selective factor Xa inhibitor. It directly inhibits factor Xa in a competitive manner with an inhibition constant in the nanomolar range. See for example, Herbert et al, J. Pharmacol. Exp. Ther. 276:1030-1038 (1996) and Nagahara et al, Eur. J. Med. Chem. 30(suppl):140s-143s (1995). As a non-peptide, synthetic factor Xa inhibitor, RPR-120844 (Rhone-Poulenc Rorer), is one of a series of novel inhibitors which incorporate 3-(S)-amino-2-pyrrolidinone as a central template. The SEL series of novel factor Xa inhibitors (SEL1915, SEL-2219, SEL-2489, SEL-2711: Selectide) are pentapeptides based on L-amino acids produced by combinatorial chemistry. They are highly selective for factor Xa and potency in the pM range.

Factor Ha inhibitors include DUP714, hirulog, hirudin, melgatran and combinations thereof. Melagatran, the active form of pro-drug ximelagatran as described in Hirsh and Weitz, Lancet, 93:203-241, (1999) and Fareed et al. Current Opinion in Cardiovascular, pulmonary and renal investigational drugs, 1:40-55, (1999).

A permanent magnet or an electromagnet can be used to generate magnetic field gradients that are directed toward the source channels, whereby the strong magnetic field gradients direct magnetically bound target components, such as cells, molecules, and/or pathogens, to migrate from the source fluid and into the transfer channels and optionally, into the collection channels. Examples of electromagnets as well as associated plates for shaping and/or concentrating the magnet field gradient are disclosed published US Patent Application No. 2009-such as Neodymium magnets, can be positioned adjacent to the collection channels 150 of the microfluidic device 100. It should be noted that other types of magnets can be used and are thus not limited to Neodymium.

Magnetic gradient configurations that ensure complete removal of the magnetic beads from the source fluid can be created. Bead trajectory in arbitrary magnetic fields and fluid flows can be predicted using simulations, which can allow finding suitable device configurations. For example, FIG. 11 shows results of computer simulations of magnetic flux concentrators designed for collection of magnetic beads within a microfluidic device described herein compared with experimental measurements of actual magnetic fields. As can be seen simulation results were in agreement with the actual data. Thus, simulations can be used to find device configurations for optimal separation efficiencies.

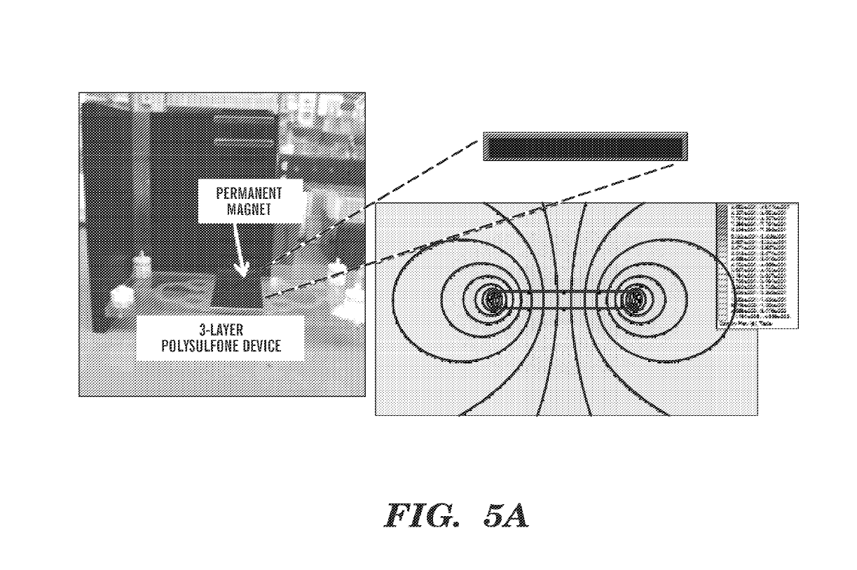

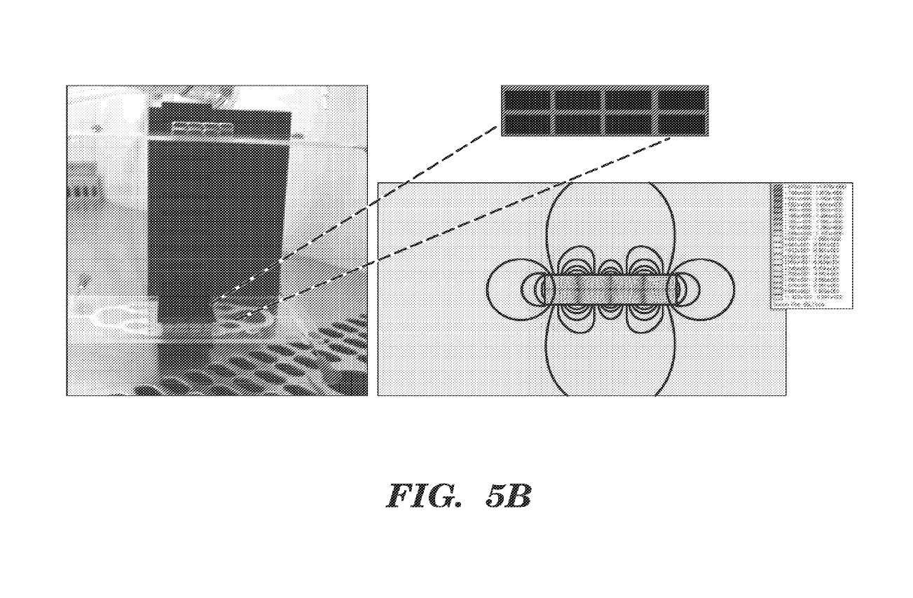

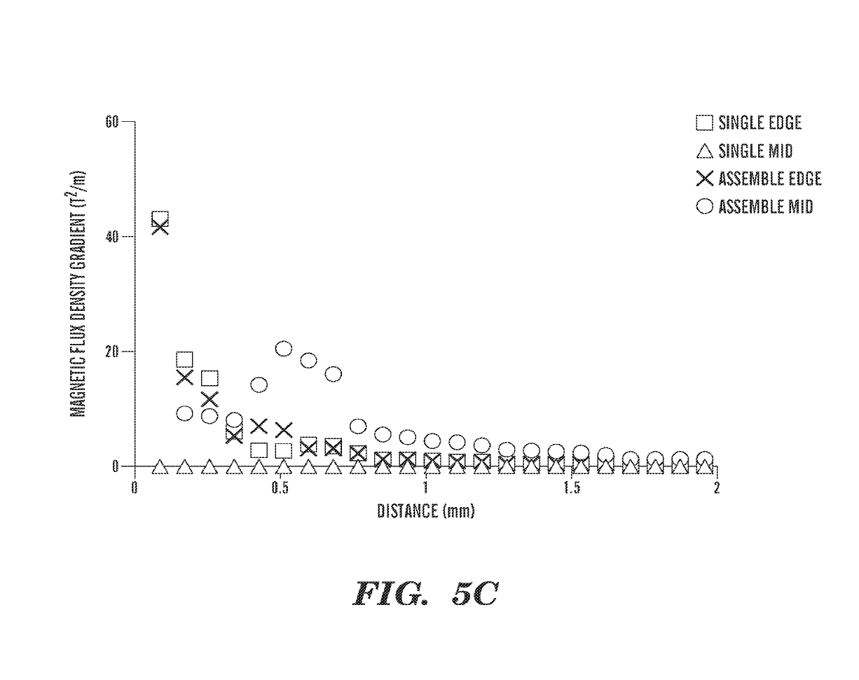

The inventors have discovered that magnetic field gradient can be improved by modifying the geometry of the magnetic source. As shown in FIGS. 5A-5C, positioning a number of smaller magnets along the collection channels provides can increase the magnetic flux density gradient by about 10³ times relative to using a single magnet adjacent to a collection channel. Accordingly, in some embodiments, two or more (e.g., two, three, four, five, six, seven, eight, nine, ten, eleven, twelve, thirteen, fourteen, fifteen or more) magnets can be positioned adjacent to a collection channel. For example, a collection channel can be subdivided into two or more (e.g., two, three, four, five, six, seven, eight, nine, ten, eleven, twelve, thirteen, fourteen, fifteen or more) adjacent sections and each section supplied with its own magnetic source.

A magnet adjacent to the collection channel can be a stack of two or more (e.g., two, three, four, five, six, seven, eight, nine, ten, eleven, twelve, thirteen, fourteen, fifteen or more) magnets. Thus, in some embodiments, two or more (e.g., two, three, four, five, six, seven, eight, nine, ten, eleven, twelve, thirteen, fourteen, fifteen or more) magnets can be positioned adjacent to a collection channel, wherein at least one, (e.g., one, two, three, four, five, six, seven, eight, nine, ten, eleven, twelve, thirteen, fourteen, fifteen, or more, including all) of the magnets is a stack of two or more (e.g., two, three, four, five, six, seven, eight, nine, ten, eleven, twelve, thirteen, fourteen, fifteen or more) magnets.

In some embodiments, the magnetic source can be a single magnet. In some embodiments, the magnetic source can be a plurality of magnets stacked together. For example, the magnetic source can be a single NdFeB N42 magnet having the dimensions 4″×1″×⅛″. In some embodiments, the magnetic source can be two or more NdFeB N42 magnets stacked together, e.g., NdFeB N42 magnets having the dimensions 2″×¼″×⅛″ and magnetized through thickness.

In some embodiments, the magnetic source can be an electromagnet constructed from a 1500 turn, 47 solenoid, and a C-shaped steel core, although other magnet designs can be used. The magnetic field concentrator, also machined from high magnetic permeability steel, can have two or more individual ridges (1×1×20 mm; w×h×l), spaced 3 mm apart, and be attached to the top side of the magnet. The total air gap between the top surface of the ridges and the opposing face of the magnet can be 5.7 mm. The electromagnetic field strength of the concentrator can be measured using a Teslameter (F. W. Bell 5080) and field gradient can be quantified by measuring the change in the field strength at a distance of 0.25 mm normal to the surface of a ridge.

A separate magnetic field gradient concentrator layer can be employed with surface ridges that run directly above the entire length of each channel to shape and/or concentrate the magnetic field gradient applied to the source channel. Since this magnetic field concentrator is not placed within the device body, multiple channels can be densely arrayed within a single device body to increase throughput. In some embodiments, further multiplexing can be achieved by stacking multiple devices vertically, interposed with multiple magnetic field gradient concentrators that are placed between each microfluidic device body inside a single electromagnet housing.

A periodic flow of the collection fluid through the collection channels can cause the magnetically bound target components in the transfer channels to flow into the collection fluid, whereby the target cells can then be removed and collected by flushing them from the device. Multiplexing can be achieved by increasing the number of channels within each device, and by stacking up multiple devices in parallel and/or serial configurations.

Depending on the fluid and device characterization, the source fluid and the collection fluid can flow through a microfluidic device at a rate ranging from about 1 mL/hr to about 2000 mL/hr. Similarly, the collection fluid can also flow through a microfluidic device at a rate ranging from about 1 mL/hr to about 2000 mL/hr.

In some embodiments, the source fluid can flow at a rate ranging from about 5 mL/hr to about 1000 mL/hr through a microfluidic device.

In some embodiment, the source fluid can flow at a flow rate that is substantially similar to venous blood flow rate of a subject.

When the source fluid is blood, the microfluidic device can support blood flow at 100 mL/hr for at least 2 hours without platelet activation or clotting by incorporating anti-fouling surfaces. In some embodiments, microfluidic device can support blood flow at 500 mL/hr for 8 hours without platelet activation or clotting. In some embodiments, microfluidic device can support blood flow at 1000 mL/hr for at least 12 hours. In some embodiments, microfluidic device can support blood flow at 1250 mL/hr for at least 24 hours. In some embodiments, the microfluidic device can support blood flow at 1500 mL/hr for at least 24 hours.

High flow rates can be obtained by connecting two or more microfluidic devices in parallel. For example, flow rates of over 800 mL/hr can be obtained by connecting 2 microfluidic devices in parallel. Flow rate of 1250 mL/hr can be obtained by connecting 3 or more microfluidic devices in parallel. These estimates are based on channels having a cross-section of 2 mm×0.16 mm. Physiologically relevant blood flows can be evaluated using a small animal pulsatile blood pump (Ismatech), which is available at the Wyss Institute and can provide flows up to 1.2 L/hr (models with larger flow rates for larger animals are also available. For example, blood can be flowed through the DLT device connected to the rat sepsis model (300 g of Wistar male rats) at flow rates ranging from 5 mL/hr to 30 mL/h. For higher mammals, such as humans, flow rates ranging from 500 mL/hr to 2000 mL/hr for continuous veno-venous circuits can be used. When used in connection with dialysis type flow circuits that use an arterivenous fistula, rates over 1 L/hr can be obtained. The optimal flow rate can be determined based on the physiologically tolerable blood flow in femoral vein/artery of animals.

The devices described herein can be fabricated from a biocompatible material. As used herein, the term “biocompatible material” refers to any polymeric material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood. Suitable biocompatible materials include derivatives and copolymers of a polyimides, poly(ethylene glycol), polyvinyl alcohol, polyethyleneimine, and polyvinylamine, polyacrylates, polyamides, polyesters, polycarbonates, and polystyrenes. A device can be fabricated from a single type of material or a combination of different types of materials.

In some embodiments, the device is fabricated from a material selected from the group consisting of aluminum, polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, the device can be fabricated from materials that are compatible with the fluids used in the system. While the plastics described herein can be used with many fluids, some materials may break down when highly acidic or alkaline fluids are used and it is recognized that the removal of the target component from the source fluid can change the composition and characteristics of the source fluid. In these embodiments, non-magnetic metals and other materials such as stainless steels, titanium, platinum, alloys, ceramics and glasses can be used.

In some embodiments, the device can be fabricated from aluminum.

In some embodiments, the device can be fabricated from FDA-approved materials.

In some embodiments, it can be desirable to use different materials in the source channel, the transfer channels and the collection channels.

A thermoplastic blood compatible material, such as the FDA-approved polysulfone polymer, can be utilized which increases the rigidity of the microfluidic device, making them easier to multiplex and to mass produce. Source channels, collection channels, and transfer channels in the thermoplastic sheet can be formed with 5 axis Microlution 5100-S micromilling machine with 1 μm resolution. Alternatively, mass replication techniques such as hot embossing or injection molding can be utilized.

The microfluidic device can be fabricated by bonding two or more individual layers of micromolded biocompatible materials. For example, the central body comprising the source fluid channels and the collection fluid channels can be first fabricated. The appropriate laminating layers can then be bonded to the fabricated central body.

Individual layers can be fabricated from the same material or different material. For example, one or more of the laminating layers of the device can be of a material different than that used for the central body of the device. For example, laminating layer of the device in contact with or next to the magnetic source can be made from a different material than rest of the device. Such a layer can be a thin polymer film. This can reduce the distance between magnetic source and source channel where the magnetic beads bound target components flow. In some embodiments, the laminating layer can be made from polypropylene, polyester, polyurethane, bi-axially oriented polypropylene (BOPP), acryl, or any combination thereof.

The laminating layer can be of any thickness. However, the inventors have discovered that thinner laminating layers allow better separation efficiencies. Accordingly, in some embodiments, the laminating layers can range in thickness from about 0.01 mm to about 10 mm. In one embodiment, the laminating layer has a thickness of about 0.1 mm.

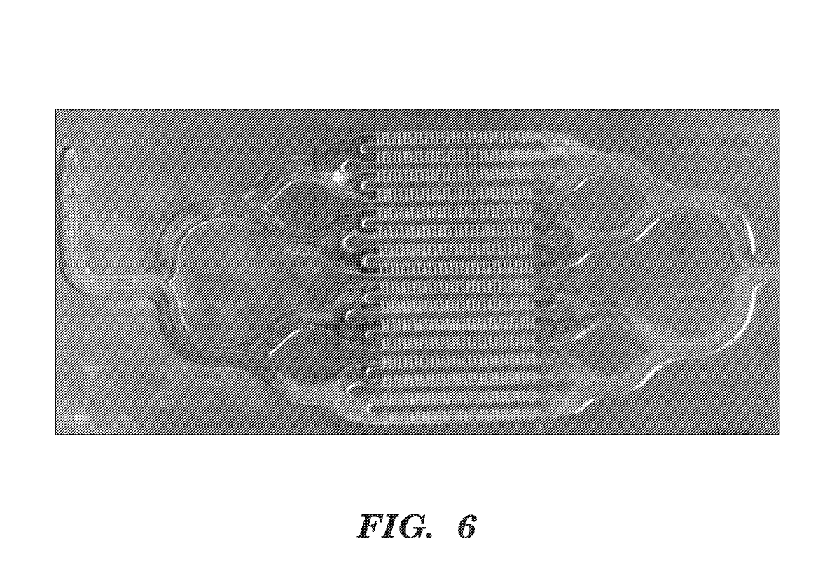

Microfluidic devices for obtaining anticoagulant SLIP surface are treated by a succession of physicochemical processes which operate in extreme conditions requiring tolerance to high temperature and mechanical stress. Accordingly, a microfluidic device can be fabricated from a material able to withstand the extreme conditions used in fabricating SLIP surface. Accordingly, in some embodiments, the central body of the microfluidic device can be fabricated from aluminum. Using aluminum for the central body allows more options to fabricate SLIPS surface on the microfluidic device channels. Aluminum provides an easy fabrication and capability to tolerate many surface modification processes, including chemical vapor deposition, chemical cleansing processes, polymer deposition at high temperatures. FIG. 6 shows a central body fabricated from aluminum.

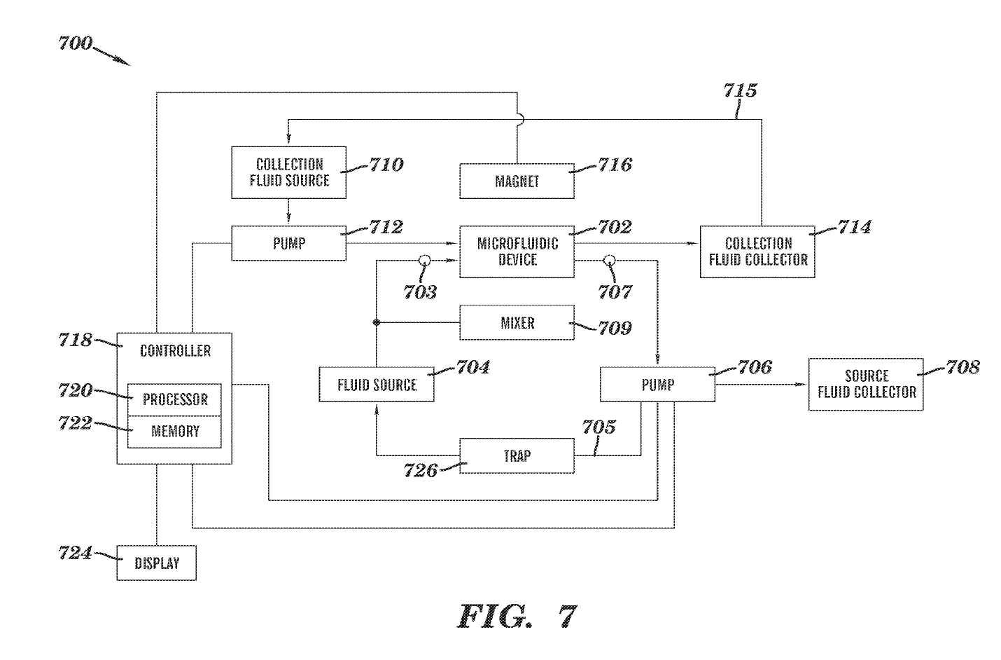

FIG. 7 illustrates a block diagram of an overall system incorporating a microfluidic device 702 described herein. In particular, the system 700 can include one or more microfluidic devices 702. It should be noted that although only one device 702 is shown in FIG. 7, more than one device 700 can be utilized as part of a system in which multiple microfluidic devices 702 can be connected to one another in serial and/or parallel fashion. Alternatively, multiple microfluidic devices 702 can be employed in a system whereby each microfluidic device 702 can be separately or individually connected between one or more fluid source(s) 704 and one or more fluid collector(s) 708.

The system in FIG. 7 can include one or more source fluid sources 704 and be configured to pump the source fluid to the microfluidic device 702. The fluid source 704 can be a human or animal, wherein the blood and/or other biological fluids are taken directly from the human or animal. The fluid source 704 can also be the source of a non-biological fluid, such as a contaminated water supply, a liquefied food source, or any fluid (liquid or gas) that can benefit from the removal of particulates or components. This can include, for example, removing contaminants from water, cleaning petroleum based lubricants and removing particulate emissions from combustion exhaust gases.

In some embodiments, a mixing component 709, such as a low-shear mixer or magnetic agitator, can be used to inject and mix magnetic particles with the source fluid prior to entering the microfluidic device 702. For example, a low-shear mixer can be used to mix magnetic particles with the source fluid. A disposable in-line mixer, which comprises a series of mixing elements having spiral baffles in a polymer tubing, can be obtained from OMEGA Engineering Inc., CT (cat #FMX8213 and FMX8214).

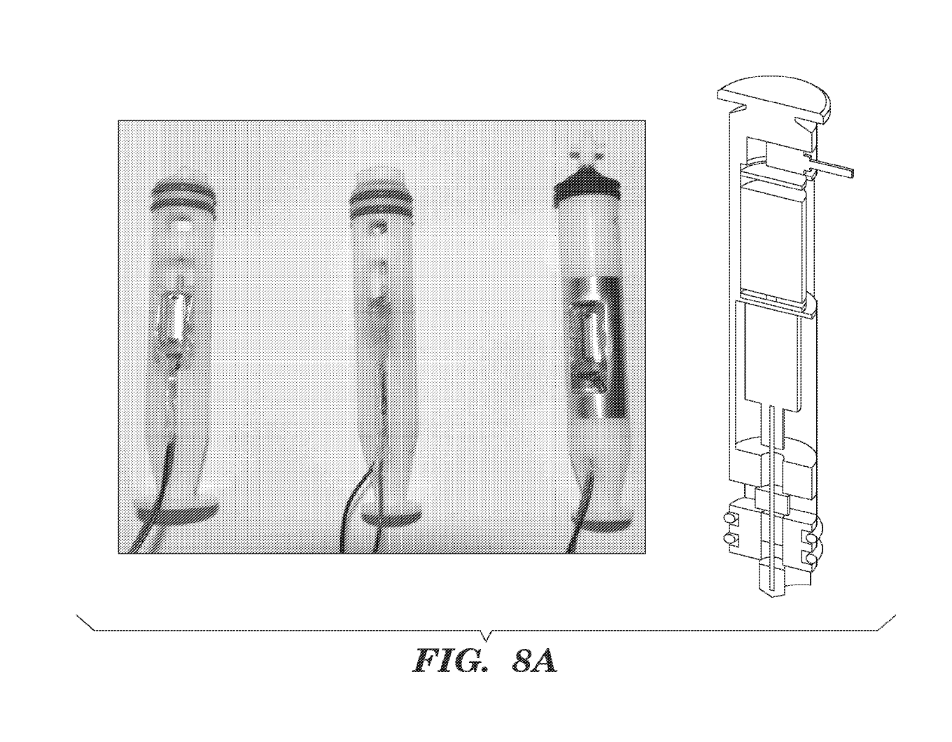

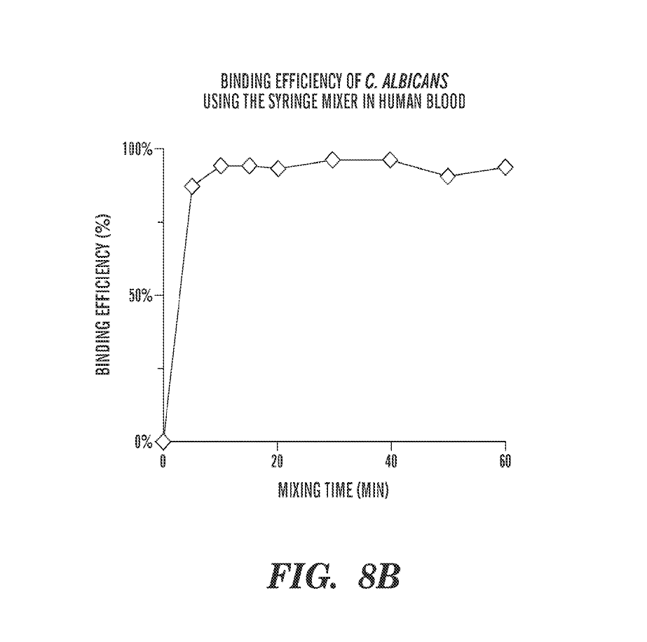

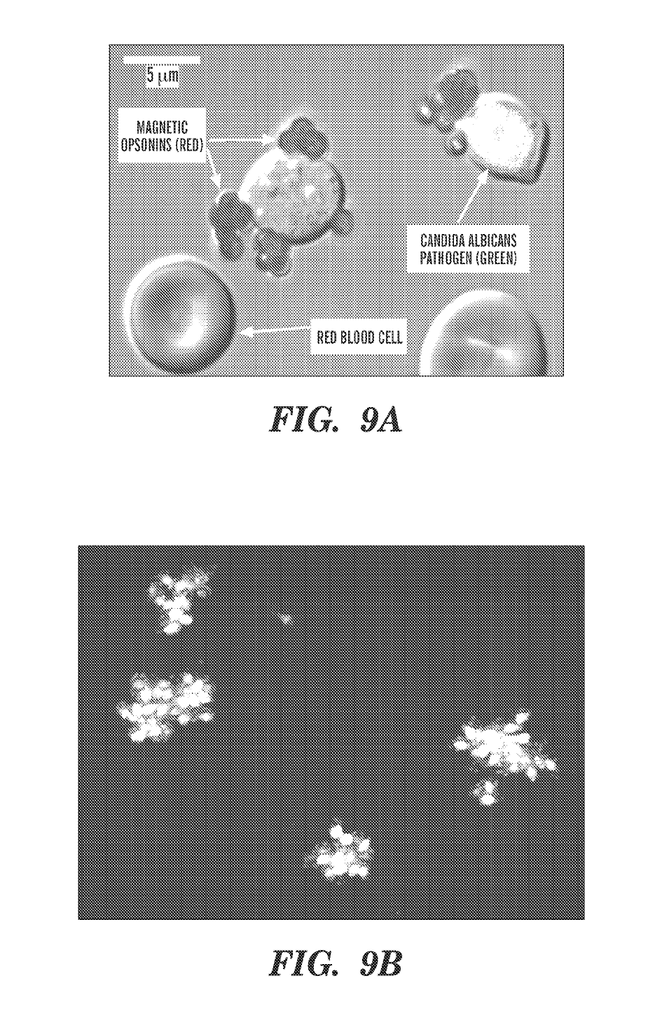

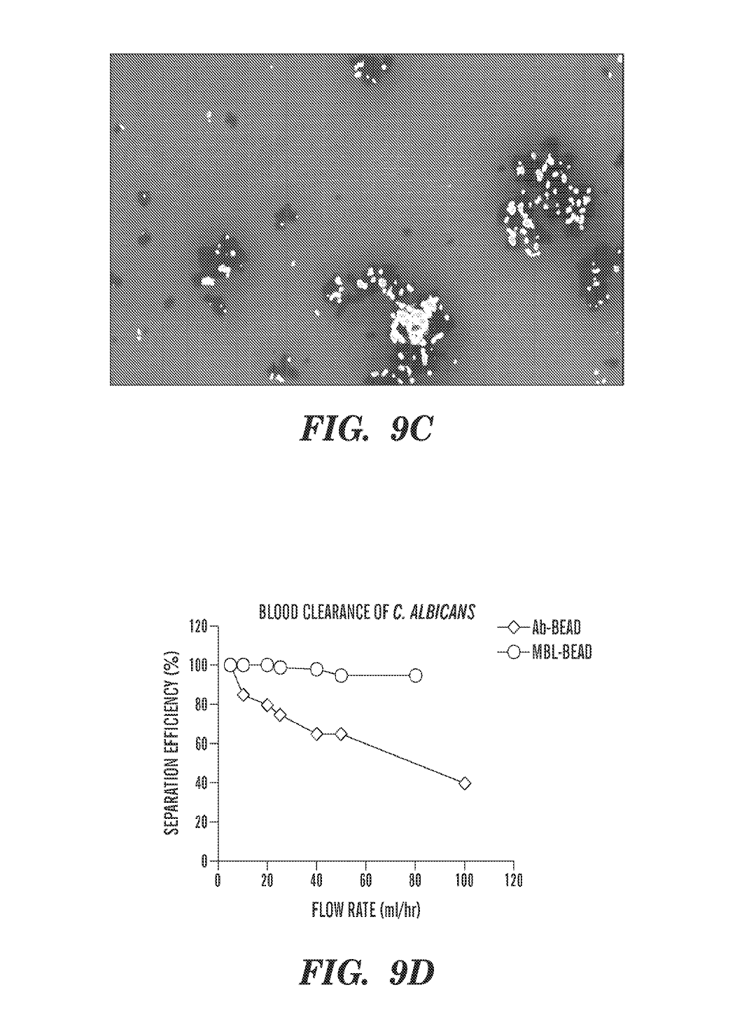

In some embodiments, the mixer is a spiral in-line mixer. In some embodiments, the mixer is a syringe mixer (FIG. 8A). The syringe mixer can accelerate magnetic particle binding to the target components, e.g., pathogens, in whole blood during pumping to obtain 90% binding of particles to pathogens in <5 minutes without inducing coagulation (FIG. 8B). As a result, pathogen clearance efficiencies in whole human blood close to 95% at flow rates above 35 mL/hr, and nearly 80% at a flow rate of more than 70 mL/hr can be achieved using magnetic beads coated with pathogen-specific antibodies. Because magnetic MBL-opsonins bind more pathogens and produce larger magnetic bead-cell clusters when bound to either fungi or E. coli compared to antibody-coated beads, eve greater pathogen clearance efficiencies close to 100% at flow rates up to 80 mL/hr can be obtained (FIGS. 9A-9D).

To accomplish efficient bead binding to the target components, e.g. pathogens, in the source fluid, e.g., blood, while maintain continuous source fluid flow at high rates, two or more syringe mixers can be connected with check-valves and they can be mounted on a single reciprocating syringe pump. While the first syringe is mixing blood with beads, the second is dispensing the last mixed batch and the cycle repeats continuously. For example, if the desired flow rate is 100 mL/hr (=1.67 mL/min) and the mixing period is 10 minutes, then each syringe can be set to draw 16.7 mL of blood on each cycle. One advantage is that that flow rates and incubation times can be adjusted separately within the syringe mixers, and as each reciprocating syringe pump can handle up to 4×60 mL syringes (240 mL capacity on each 10 minute cycle). With multiple setups linked in parallel, a continuous flow rate of 1440 mL/hr can be produced. In addition, opsonin coated beads be reutilized after they are magnetically collected so that they can be recycled to provide continuous pathogen capture capabilities with a single device. To accomplish this, engineered MBL can be used or unbound magnetic particles can collected from pathogen bound ones using flow filtration across a 2 μm track-etched membrane; unbound beads that pass through this size pore can be reused.

Magnetic particles can be continually infused into the mixer 709 at an optimized rate. At this stage, the magnetic particles will selectively bind to the target components in the source fluid and confer magnetic mobility only to these target components. As the source fluid flows from the mixer 709 into the microfluidic device 702, the low aspect ratio of the microfluidic channel effectively flattens out the geometry of the source fluid to maximize the area of exposure to the magnetic field gradients, as well as to minimize the distance that magnetically bound pathogens travel to reach the transfer channels on their way to the collection channel. The transfer channels and source fluid channel(s) can be pre-filled with the collection fluid, such as saline, although other compatible fluids, such as the collection fluids described herein can also be used.

As shown in FIG. 7 one or more pumps 706 can be connected to the microfluidic device 702 causing the fluid to flow through the microfluidic device 702. It should be noted that although the pump 706 is shown downstream from the microfluidic device 702, a pump 706 can be additionally/alternatively located upstream from the microfluidic device 702. In one embodiment, the pump 706 can be connected to one or more source fluid collectors 708 where some or all of the exit fluid is collected and stored.

In one embodiment where the source fluid is a biological fluid, the biological fluid that passes through the microfluidic device 702 can be returned to the human or animal from where the biological fluid was taken. Additionally or alternatively, the pump 706 can be connected to the fluid source 704 (via line 705), whereby the exiting fluid can be recirculated to the fluid source 104 to be processed by the microfluidic device 702. The pump 706 can be an electronic, automatically-controlled pump or a manually-operated pump. Alternatively, the fluid source can be elevated to allow gravity to push, with or without the assistance of a pump, the source fluid through the microfluidic device 702. The microfluidic system 700 can include one or more flow valves 703, 707 connected at the inlet and/or the outlet of the microfluidic device 702 to allow the flow of the source fluid to be stopped, for example, during the time when the collection fluid flows through the collection channel.

As shown in FIG. 7, one or more air bubble traps 726 can be connected to the microfluidic device 702 causing any air bubbles in the fluid lines to be trapped or removed from the fluid that flow through the microfluidic device 702. It should be noted that although the trap 726 is shown downstream from the microfluidic device 702, a trap 726 can be additionally/alternatively located upstream from the microfluidic device 702. In one embodiment, the trap 726 can be connected to the source fluid collector 708 where some or all of the exit fluid is collected and stored.

In one embodiment, the microfluidic device 702 can also be connected to one or more collection fluid sources 710 which supply the collection fluid to the microfluidic device 702. In an embodiment, one or more pumps 712 can be connected to the collection fluid source 710 to supply the collection fluid to the microfluidic device 702. It should be noted that, as with pump 706, one or more pumps 712 can be additionally/alternatively located downstream from the microfluidic device 702 instead of upstream, as shown in FIG. 7. It should also be noted that the pump 712 is optional and a syringe or other appropriate device (or gravity) can be used to drive the collection fluid through the microfluidic device 702 to the collection fluid collector 114 or an inline analysis or detection device.

In one embodiment, the microfluidic device 702 can be connected to a collection fluid collector 714, whereby exiting collection fluid is stored in the collector 714. Additionally or alternatively, the collector 714 can be connected to the collection fluid source 710 (via line 715), whereby the exiting collection fluid can return to the collection fluid source 710 to be recirculated through to the microfluidic device 702. Prior to returning the collection fluid to the collection fluid source 710, the collection fluid can be processed to remove the magnetically bound target components, such as by filtering or using magnetic separating techniques.

As shown in FIG. 7, one or more magnetic sources 716 can be positioned proximal to the microfluidic device 702. The magnetic source 716 aid in removing magnetic particles that are attached to target components in the source fluid, as discussed herein.

The system 700 can also include one or more controllers 718 coupled to one or more of the components in the system. The controller 718 preferably includes one or more processors 720 and one or more local/remote storage memories 722. A display 724 can be coupled to the controller 718 to provide a user interface to control the operation of the system and display resultant, operational and/or performance data in real time to the user. The controller 718 can be optionally connected to pump 706 and/or pump 712 to individually or collectively control operational parameters of these components, such the flow rates and/or initiating and terminating flow of the respective fluids in and out of the microfluidic device 702. Optionally, the controller 718 can be connected to the fluid sources 704, 710, the valves 703, 707, the mixer component 709 and/or the collectors 708, 714 to operate valves in these components and/or to selectively dispense respective fluids or magnetic beads in a controlled manner within the system. Optionally, the controller 718 can be connected to the one or more magnetic sources 716 to selectively control power, voltage and/or current supplied to the magnetic sources 716 to control and adjust the magnetic field gradients in order to control the performance of the microfluidic device 702. It is also possible for the controller 718 to selectively position and control the force levels of the magnet field gradients at desired distances with respect to the microfluidic device 702 to selectively control the magnetic field gradient applied to the channels of the microfluidic device 702. Although not shown, the controller 718 can be connected to various sensors in the microfluidic device 702 and/or other components in the system 700 to monitor and analyze the behavior and interaction of the fluids and/or target components traveling in the system 700. The controller 718 can be a personal computer including software and hardware interfaces connected to the pumps, valves and sensors to control the operation of the system 700. Alternatively, controller 718 can be dedicated micro controller specifically designed or programmed with dedicated software to interface with the pumps, valves and sensors to control the system 700. It should be noted that the system shown in FIG. 7 is exemplary and that additional, other or less components may be employed without departing from the inventive concepts herein.

In some embodiments, the system 700 can include sensors that monitor the migration of the target components through the transfer channel 714 into the collection channel 150 in order to determine how to control the flow in the collection channel 150 to remove the accumulated target components. The sensor can be one or more optical sensors that detect the accumulation of target components as they block light projected through the transfer channel or the collection channel onto the sensor or detect light reflected by target components. The optical detector can be a simple photodiode or a more complex imaging device, such as a CCD based camera. When the sensor detects that a predefined amount of target components has accumulated in the transfer channel or the collection channel, the signal from the sensor to the controller can cause the controller to change (e.g. increase) the flow in the collection channel, or initiate the flushing operation. At the same time the controller can stop the pump 106 and/or operate the valves 703, 707 to stop or reduce the flow of the source fluid through the source channel 140.

The microfluidic devices and systems described herein exhibits simplicity of design and fabrication, very high flow throughput, higher separation efficiency, and minimal blood alteration (e.g., clots, loss, dilution). This simple design also obviates the need for complex control of two fluids and maintenance of a stable border between adjacent laminar flow streams, and simplifies multiplexing. It will likely be less expensive and simpler to manufacture and assemble, and exhibit a similar or enhanced ability to be integrated into existing blood filtration biomedical devices such as those used for continuous renal replacement therapy (CRRT), extracorporeal membrane oxygenation (ECMO), and continuous veno-venous hemofiltration (CVVH).

The microfluidic device 702 and the magnet 716 can be located in a housing, i.e., device housing. The device housing can be used to connect and physically assemble multiple microfluidic devices and magnetic sources. The housing can have a scalable assembly that can accommodate 1 or more, (e.g., one, two, three, four, five, six, seven, eight, nine, ten, eleven, twelve, thirteen, fourteen, fifteen or more) sets of microfluidic devices and magnetic sources. For example, individual permanent magnets (such as NIB magnets) with alternating poles can be fixed in the housing such that portions of the magnets can be left exposed like fins in heat sink. The magnetic fins can be spaced appropriately to fit between multiplexed microfluidic devices and enable separation of magnetic particle bound target components on both sides.

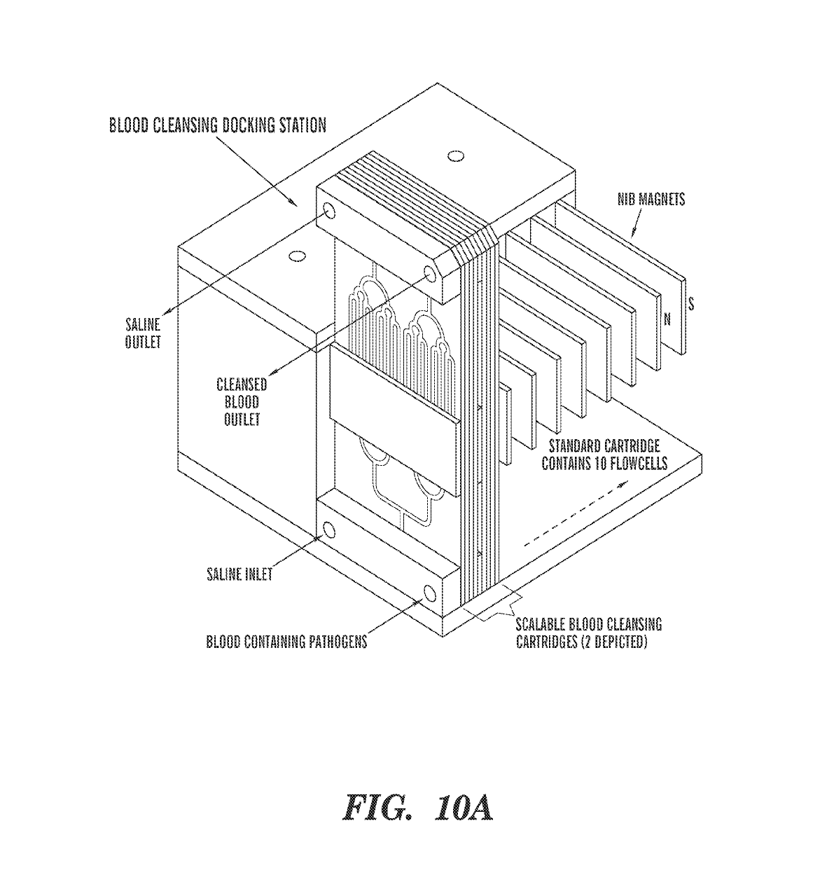

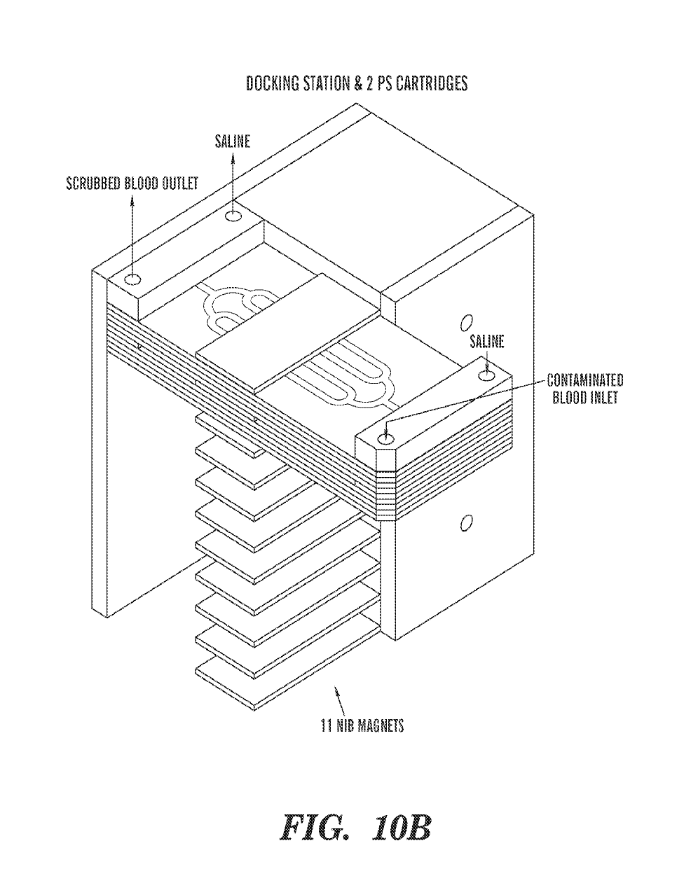

The housing can be made from any non-magnetic material. For example, housing can be made from aluminum, plastic, plastic (e.g. Darlin plastic), and the like. FIGS. 10A and 10B show schematic representations of docking stations.

As used herein, the term “source fluid” refers to any flowable material that comprises the target component. Without wishing to be bound by theory, the source fluid can be liquid (e.g., aqueous or non-aqueous), supercritical fluid, gases, solutions, suspensions, and the like.

In some embodiments, the source fluid is a biological fluid. The terms “biological fluid” and “biofluid” are used interchangeably herein and refer to aqueous fluids of biological origin, including solutions, suspensions, dispersions, and gels, and thus may or may not contain undissolved particulate matter. Exemplary biological fluids include, but are not limited to, blood (including whole blood, plasma, cord blood and serum), lactation products (e.g., milk), amniotic fluids, peritoneal fluid, sputum, saliva, urine, semen, cerebrospinal fluid, bronchial aspirate, perspiration, mucus, liquefied feces, synovial fluid, lymphatic fluid, tears, tracheal aspirate, and fractions thereof.

Another example of a group of biological fluids are cell culture fluids, including those obtained by culturing or fermentation, for example, of single- or multi-cell organisms, including prokaryotes (e.g., bacteria) and eukaryotes (e.g., animal cells, plant cells, yeasts, fungi), and including fractions thereof.

Yet another example of a group of biological fluids are cell lysate fluids including fractions thereof. For example, cells (such as red blood cells, white blood cells, cultured cells) may be harvested and lysed to obtain a cell lysate (e.g., a biological fluid), from which molecules of interest (e.g., hemoglobin, interferon, T-cell growth factor, interleukins) may be separated with the aid of the present invention.

Still another example of a group of biological fluids are culture media fluids including fractions thereof. For example, culture media comprising biological products (e.g., proteins secreted by cells cultured therein) may be collected and molecules of interest separated therefrom with the aid of the present invention.

In some embodiments, the source fluid is a non-biological fluid. As used herein, the term “non-biological fluid” refers to any aqueous, non-aqueous or gaseous sample that is not a biological fluid as the term is defined herein. Exemplary non-biological fluids include, but are not limited to, water, salt water, brine, organic solvents such as alcohols (e.g., methanol, ethanol, isopropyl alcohol, butanol etc. . . . ), saline solutions, sugar solutions, carbohydrate solutions, lipid solutions, nucleic acid solutions, hydrocarbons (e.g. liquid hydrocarbons), acids, gasolines, petroleum, liquefied samples (e.g., liquefied foods), gases (e.g., oxygen, CO₂, air, nitrogen, or an inert gas), and mixtures thereof.

In some embodiments, the source fluid is a media or reagent solution used in a laboratory or clinical setting, such as for biomedical and molecular biology applications. As used herein, the term “media” refers to a medium for maintaining a tissue or cell population, or culturing a cell population (e.g. “culture media”) containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. The media can include media to which cells have been already been added, i.e., media obtained from ongoing cell culture experiments, or in other embodiments, be media prior to the addition of cells.

As used herein, the term “reagent” refers to any solution used in a laboratory or clinical setting for biomedical and molecular biology applications. Reagents include, but are not limited to, saline solutions, PBS solutions, buffer solutions, such as phosphate buffers, EDTA, Tris solutions, and the like. Reagent solutions can be used to create other reagent solutions. For example, Tris solutions and EDTA solutions are combined in specific ratios to create “TE” reagents for use in molecular biology applications.

The source fluid can flow at any desired flow rate through the microchannel. For example, the source fluid can flow at a rate of 1 mL/hr to 2000 mL/hr through source channel.

As used herein, the term “collection fluid” refers to any flowable material that can be used for collecting the target component magnetic particle complexes. Like source fluids, collection fluid can also be liquid (e.g., aqueous or non-aqueous), supercritical fluid, gases, solutions, suspensions, and the like.

Choice of collection fluid depends on the particular application and the source fluid. Generally, the collection fluid is chosen so that it is compatible with the source fluid and/or the target component-magnetic particle complex. As used herein, compatibility with the source fluid means that collection fluid has similar density, Cₚ, enthalpy, internal energy, viscosity, Joule-Thomson coefficient, specific volume, Cᵥ, entropy, thermal conductivity, isotonicity, and/or surface tension to the source fluid. In some embodiments, the collection fluid is miscible with the source fluid. In some other embodiments, the collection fluid is not miscible with the source fluid.

In accordance with the invention, the collection fluid can be a fluid that is compatible with the source fluid and cleansing process. Thus, the collection fluid can be any fluid that will not contaminate the source fluid when mixed therein. In some embodiments, the collection fluid can be the same or similar composition as the source fluid. For example, where the source fluid is a biofluid, a compatible collection fluid such as an isotonic saline solution, a saline solution containing serum, such as fetal bovine serum, a physiological salt solution, a buffer, a cell culture media, or the like. Generally, the collection fluid should be isotonic compared to the biofluid to minimize diffusional mass transfer and osmotic damage to cells. Although collection fluid does not need to match the viscosity of the source fluid for proper operations, similar viscosities can minimize shear mixing. When the source fluid is a biological fluid, the collection fluid is generally a non-toxic fluid. Biocompatible or injectable solutions are desirable, especially for therapeutic applications involving human patients. In some embodiments, the collection fluid is a biological fluid, a biocompatible fluid or a biological fluid substitute.

As used herein, the term “biocompatible fluid” refers to any fluid that is appropriate for infusion into a subject's body, including normal saline and its less concentrated derivatives, Ringer's lactate, and hypertonic crystalloid solutions; blood and fractions of blood including plasma, platelets, albumin and cryoprecipitate; blood substitutes including hetastarch, polymerized hemoglobin, perfluorocarbons; LIPOSYN (lipid emulsion used for intravenous feeding); blood or serum components reconstituted with saline or sterile water, and combinations thereof.

In some embodiments, the collection fluid includes one or more fluids selected from the group consisting of biological fluids, physiologically acceptable fluids, biocompatible fluids, water, organic solvents such as alcohols (e.g., methanol, ethanol, isopropyl alcohol, butanol etc. . . . ), saline solutions (e.g., isotonic saline solution), sugar solutions, hydrocarbons (e.g. liquid hydrocarbons), acids, and mixtures thereof. In some embodiments, the collection fluid is the source fluid without the target component. In some embodiments, the collection fluid is a gas such as oxygen, CO₂, air, nitrogen, or an inert gas.

In some embodiments, the collection fluid is saline or is formed from saline.

The collection fluid can flow at the same or different flow rates compared to the source fluid. For example, the collection fluid can flow at a rate of 1 mL/hr to 1000 L/hr through collection channel 150. In addition, the pressure applied to the collection fluid in the microfluidic device 100 can be controlled to prevent the mixing or loss of the source fluid. For example, the collection fluid can be maintained at a lower pressure than the source fluid to prevent the collection fluid from entering the transfer channels 160 and mixing with the source fluid. Alternatively, the collection fluid, being compatible with the source fluid, can be maintained at a higher pressure than the source fluid allowing some collection fluid to enter the transfer channels 160 to prevent the entry and loss of the source fluid into the collection channel 150. In one embodiment and as described further below, the flow of the collection fluid can be cycled between flowing and stagnant or nearly stagnant. For example, the collection fluid can be stationary or stagnant and maintain a relatively high pressure for a period of time sufficient for target components to accumulate in the collection channel 150 and/or the transfer channels 160 and, when a determined amount of target components have accumulated (e.g., as a function of time or volume), the collection fluid can be cycled into the flowing state at the same pressure to flush out the target components and replace the collection channel 150 with cleaner collection fluid without altering the remaining source fluid. The periodic flushing operation can lower the pressure in the collection channel 150 to draw the fluid in the transfer channels into the collection channel 150 to facilitate flushing of the target components. During the flushing operation, the source fluid can be stopped, stagnant, or nearly stagnant to minimize or prevent the loss of source fluid into the transfer channel 160 and/or the collection channel 150.

The magnetic particles can be of any size or shape. For example, magnetic particles can be spherical, rod, elliptical, cylindrical, disc, and the like. In some embodiments, magnetic particles having a substantially spherical shape can be used. Particles of defined surface chemistry can be used to minimize chemical agglutination and non-specific binding.

As used herein, the term “magnetic particle” refers to a nano- or micro-scale particle that is attracted or repelled by a magnetic field gradient or has a non-zero magnetic susceptibility. The term “magnetic particle” also includes magnetic particles that have been conjugated with affinity molecules. The magnetic particles can be paramagnetic or super-paramagnetic particles. In some embodiments, the magnetic particles can be superparamagnetic. Magnetic particles are also referred to as beads herein.

In some embodiments, magnetic particles having a polymer shell can be used to protect the target component from exposure to iron. For example, polymer coated magnetic particles can be used to protect target cells from exposure to iron. In some embodiments, the magnetic particles or beads can be selected to be compatible with the fluids being used, so as not to cause undesirable changes to the source fluid. For example, for biological fluids, the magnetic particles can made from well know biocompatible materials.

The magnetic particles can range in size from 1 nm to 1 mm. For example, magnetic particles can be about 250 nm to about 250 μm in size. In some embodiments, magnetic particle can be from about 0.1 μm to about 50 μm in size. In some embodiments, magnetic particle can be from about 0.1 μm to about 10 μm in size. In some embodiments, magnetic particle can be from about 50 nm to about 5 μm in size. In some embodiments, magnetic particle can be from about 100 nm to about 1 μm in size. In some embodiments, magnetic particle can be about 1 μm in size. In some embodiments, magnetic particle can be about 114 nm in size. In some embodiments, magnetic beads cab be about 50 nm, 2.8 μm or about 4.5μ, in size.

The inventors have also discovered that different target components, e.g., pathogens, bind with different efficiencies to magnetic particles of different sizes. Accordingly, magnetic particles of different sizes can be used together. This can enhance target component binding the magnetic particle or allow separating different target components from the source fluid.

In some embodiments, the magnetic particle can be a magnetic nano-particle or magnetic microparticle. Magnetic nanoparticles are a class of nanoparticle which can be manipulated using magnetic field. Such particles commonly consist of magnetic elements such as iron, nickel and cobalt and their chemical compounds. Magnetic nano-particles are well known and methods for their preparation have been described in the art, for example in U.S. Pat. Nos. 6,878,445; 5,543,158; 5,578,325; 6,676,729; 6,045,925 and 7,462,446, and U.S. Pat. Pub. Nos.: 2005/0025971; 2005/0200438; 2005/0201941; 2005/0271745; 2006/0228551; 2006/0233712; 2007/01666232 and 2007/0264199, contents of all of which are herein incorporated by reference in their entirety.

Magnetic particles are easily and widely available commercially, with or without functional groups capable of binding to affinity molecules. Suitable superparamagnetic particles are commercially available such as from Dynal Inc. of Lake Success, N.Y.; PerSeptive Diagnostics, Inc. of Cambridge, Mass.; Invitrogen Corp. of Carlsbad, Calif.; Cortex Biochem Inc. of San Leandro, Calif.; and Bangs Laboratories of Fishers, Ind. Magnetic beads or particles are also available from Miltenyi Biotech (50 nm magnetic nanoparticles), and Invitrogen (2.8 um or 4.5 um magnetic microbeads). In some embodiments, magnetic particles are Dynal Magnetic beads such as MyOne Dynabeads.

The surfaces of the magnetic particles can be functionalized to include binding molecules that bind selectively with the target component. These binding molecules are also referred to as affinity molecules herein. The binding molecule can be bound covalently or non-covalently (e.g. adsorption of molecule onto surface of the particle) to each magnetic particle. The binding molecule can be selected such that it can bind to any part of the target component that is accessible. For example, the binding molecule can be selected to bind to any antigen of a pathogen that is accessible on the surface, e.g., a surface antigen.

As used herein, the term “binding molecule” or “affinity molecule” refers to any molecule that is capable of binding a target component. Representative examples of affinity molecules include, but are not limited to, antibodies, portions of antibodies, antigen binding fragments of antibodies, antigens, opsonins, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); and biological, chemical or other molecules that have affinity for another molecule, such as biotin and avidin. The binding molecules need not comprise an entire naturally occurring molecule but can consist of only a portion, fragment or subunit of a naturally or non-naturally occurring molecule, as for example the Fab fragment of an antibody. The binding molecule may further comprise a marker that can be detected.

In some embodiments, the affinity molecule can comprise an opsonin or a fragment thereof. The term “opsonin” as used herein refers to naturally-occurring and synthetic molecules which are capable of binding to or attaching to the surface of a microbe or a pathogen, of acting as binding enhancers for a process of phagocytosis. Examples of opsonins which can be used in the engineered molecules described herein include, but are not limited to, vitronectin, fibronectin, complement components such as C1q (including any of its component polypeptide chains A, B and C), complement fragments such as C3d, C3b and C4b, mannose-binding protein, conglutinin, surfactant proteins A and D, C-reactive protein (CRP), alpha2-macroglobulin, and immunoglobulins, for example, the Fc portion of an immunoglobulin.

In some embodiments, the affinity molecule comprises a carbohydrate recognition domain or a carbohydrate recognition portion thereof. As used herein, the term “carbohydrate recognition domain” refers to a region, at least a portion of which, can bind to carbohydrates on a surface of a pathogen.

In some embodiments, affinity molecule comprises a lectin or a carbohydrate recognition or binding fragment or portion thereof. The term “lectin” as used herein refers to any molecules including proteins, natural or genetically modified, that interact specifically with saccharides (i.e., carbohydrates). The term “lectin” as used herein can also refer to lectins derived from any species, including, but not limited to, plants, animals, insects and microorganisms, having a desired carbohydrate binding specificity. Examples of plant lectins include, but are not limited to, the Leguminosae lectin family, such as ConA, soybean agglutinin, and lentil lectin. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some embodiments, the carbohydrate recognition domain can be derived from a C-type lectin, or a fragment thereof.

Collectins are soluble pattern recognition receptors (PRRs) belonging to the superfamily of collagen containing C-type lectins. Exemplary collectins include, without limitations, mannan-binding lectin (MBL) or mannose-binding protein, surfactant protein A (SP-A), surfactant protein D (SP-D), collectin liver 1 (CL-L1), collectin placenta 1 (CL-P1), conglutinin, collectin of 43 kDa (CL-43), collectin of 46 kDa (CL-46), and a fragment thereof.

In some embodiments, the affinity molecule comprises the full amino acid sequence of a carbohydrate-binding protein.

Generally, any art-recognized recombinant carbohydrate-binding proteins or carbohydrate recognition domains can be used in affinity molecules. For example, recombinant manose-binding lectins, e.g., but not limited to, the ones disclosed in the U.S. Pat. Nos. 5,270,199; 6,846,649; and U.S. Patent Application No. US 2004/0,229,212, content of all of which is incorporated herein by reference, can be used in constructing an affinity molecule.

In some embodiments, affinity molecule comprises a mannose-binding lectin (MBL) or a carbohydrate binding fragment or portion thereof. Mannose-binding lectin, also called mannose binding protein (MBP), is a calcium-dependent serum protein that can play a role in the innate immune response by binding to carbohydrates on the surface of a wide range of microbes or pathogens (viruses, bacteria, fungi, protozoa) where it can activate the complement system. MBL can also serve as a direct opsonin and mediate binding and uptake of pathogens by tagging the surface of a pathogen to facilitate recognition and ingestion by phagocytes.

In some embodiments, the affinity molecule comprises an MBL or an engineered form of MBL (FcMBL: IgG Fc fused to mannose binding lectin, or Akt-FcMBL: IgG Fc fused to mannose binding lectin with the N-terminal amino acid tripeptide of sequence AKT (alanine, lysine, threonine)) as described in PCT Application No. PCT/US2011/021603, filed Jan. 19, 2011 and U.S. Provisional Application No. 61/508,957, filed Jul. 18, 2011, content of both of which is incorporated herein by reference. Amino acid sequences for MBL and engineered MBL are:

| | |
|---|---|
| (i) | MBL full length (SEQ ID NO. 1): MSLFPSLPLL LLSMVAASYS ETVTCEDAQK |
| | TCPAVIACSS PGINGFPGKD GRDGTKGEKG EPGQGLRGLQ GPPGKLGPPG |
| | NPGPSGSPGP KGQKGDPGKS PDGDSSLAAS ERKALQTEMA RIKKWLTFSL |
| | GKQVGNKFFL TNGEIMTFEK VKALCVKFQA SVATPRNAAE NGAIQNLIKE |
| | EAFLGITDEK TEGQFVDLTG NRLTYTNWNE GEPNNAGSDE DCVLLLKNGQ |
| | WNDVPCSTSH LAVCEFPI |
| (ii) | MBL without the signal sequence (SEQ ID NO. 2): ETVTCEDAQK TCPAVIACSS |
| | PGINGFPGKD GRDGTKGEKG EPGQGLRGLQ GPPGKLGPPG NPGPSGSPGP |
| | KGQKGDPGKS PDGDSSLAAS ERKALQTEMA RIKKWLTFSL GKQVGNKFFL |
| | TNGEIMTFEK VKALCVKFQA SVATPRNAAE NGAIQNLIKE EAFLGITDEK |
| | TEGQFVDLTG NRLTYTNWNE GEPNNAGSDE DCVLLLKNGQ |
| | WNDVPCSTSH LAVCEFPI |
| (iii) | Truncated MBL (SEQ ID NO. 3): AASERKALQT EMARIKKWLT FSLGKQVGNK |
| | FFLTNGEIMT FEKVKALCVK FQASVATPRN AAENGAIQNL IKEEAFLGIT |
| | DEKTEGQFVD LTGNRLTYTN WNEGEPNNAG SDEDCVLLLK |
| | NGQWNDVPCS TSHLAVCEFP I |
| (iv) | Carbohydrate recognition domain (CRD) of MBL (SEQ ID NO. 4): VGNKFFLTNG |
| | EIMTFEKVKA LCVKFQASVA TPRNAAENGA IQNLIKEEAF LGITDEKTEG |
| | QFVDLTGNRL TYTNWNEGEP NNAGSDEDCV LLLKNGQWND |
| | VPCSTSHLAV CEFPI |
| (v) | Neck + Carbohydrate recognition domain of MBL (SEQ ID NO. 45): PDGDSSLAAS |
| | ERKALQTEMA RIKKWLTFSL GKQVGNKFFL TNGEIMTFEK VKALCVKFQA |
| | SVATPRNAAE NGAIQNLIKE EAFLGITDEK TEGQFVDLTG NRLTYTNWNE |
| | GEPNNAGSDE DCVLLLKNGQ WNDVPCSTSH LAVCEFPI |
| (vi) | FcMBL.81 (SEQ ID NO. 6): EPKSSDKTHT CPPCPAPELL GGPSVFLFPP |
| | KPKDTLMISR TPEVTCVVVD VSHEDPEVKFNWYVDGVEVH NAKTKPREEQ |
| | YNSTYRVVSV LTVLHQDWLN GKEYKCKVSN KALPAPIEKT ISKAKGQPRE |
| | PQVYTLPPSR DELTKNQVSL TCLVKGFYPS DIAVEWESNG |
| | QPENNYKTTPPVLDSDGSFF LYSKLTVDKS RWQQGNVFSC SVMHEALHNH |
| | YTQKSLSLSP GAPDGDSSLAASERKALQTE MARIKKWLTF SLGKQVGNKF |
| | FLTNGEIMTF EKVKALCVKF QASVATPRNA AENGAIQNLI KEEAFLGITD |
| | EKTEGQFVDL TGNRLTYTNW NEGEPNNAGS DEDCVLLLKN |
| | GQWNDVPCST SHLAVCEFPI |
| (vii) | Akt-FcMBL (SEQ ID NO. 7): AKTEPKSSDKTHT CPPCPAPELL GGPSVFLFPP |
| | KPKDTLMISR TPEVTCVVVD VSHEDPEVKF NWYVDGVEVH NAKTKPREEQ |
| | YNSTYRVVSV LTVLHQDWLN GKEYKCKVSN KALPAPIEKT ISKAKGQPRE |
| | PQVYTLPPSR DELTKNQVSL TCLVKGFYPS DIAVEWESNG QPENNYKTTP |
| | PVLDSDGSFF LYSKLTVDKS RWQQGNVFSC SVMHEALHNH YTQKSLSLSP |
| | GAPDGDSSLA ASERKALQTE MARIKKWLTF SLGKQVGNKF FLTNGEIMTF |
| | EKVKALCVKF QASVATPRNA AENGAIQNLI KEEAFLGITD EKTEGQFVDL |
| | TGNRLTYTNW NEGEPNNAGS DEDCVLLLKN GQWNDVPCST SHLAVCEFPI |
| (viii) | FcMBL.111 (SEQ ID NO. 8): EPKSSDKTHT CPPCPAPELL GGPSVFLFPP |
| | KPKDTLMISR TPEVTCVVVD VSHEDPEVKF NWYVDGVEVH NAKTKPREEQ |
| | YNSTYRVVSV LTVLHQDWLN GKEYKCKVSN KALPAPIEKT ISKAKGQPRE |
| | PQVYTLPPSR DELTKNQVSL TCLVKGFYPS DIAVEWESNG QPENNYKTTP |
| | PVLDSDGSFF LYSKLTVDKS RWQQGNVFSC SVMHEALHNH YTQKSLSLSP |
| | GATSKQVGNKF FLTNGEIMTF EKVKALCVKF QASVATPRNA AENGAIQNLI |
| | KEEAFLGITD EKTEGQFVDL TGNRLTYTNW NEGEPNNAGS DEDCVLLLKN |
| | GQWNDVPCST SHLAVCEFPI |

In some embodiments, microbe-targeting molecule comprises an amino acid sequence selected from SEQ ID NO. 1-SEQ ID NO. 8.

The affinity molecules comprising lectins or modified versions thereof can act as broad-spectrum pathogen binding molecules. Accordingly, devices and methods utilizing lectins (e.g., MBL and genetically engineered version of MBL (FcMBL and Akt-FcMBL)) as broad-spectrum pathogen binding molecules to capture or separate pathogens can be carried out without identifying the pathogen.

In pathogen binding studies carried out in vitro using opsonin coated magnetic beads (1 μm) in diameter that restored the natural multivalency of MBL, both the native and engineered forms of MBL were found to bind a similar wide range of living pathogens including (C. albicans, P. aeriginosa, B. subtilis, E. coli, B. cenocepacia, Klebsiella, S. epidermidis) when magnetically isolated from a saline solution, a serum substitute (saline containing serum albumin) or whole blood. Using fungal pathogens (C. albicans), the inventors have been able to achieve 97.5±3.2% isolation efficiency after only 10 minutes of binding in the serum substitute.

The engineered MBL (FcMBL or AKT-FcMBL) can be produced in 293F cells by transient transfection. A stable expression system in CHO-K1 cells can be developed to provide large amounts of reagent (>10 pg/cell/day: ˜1 gm/L). After selecting clones, the protein product can be tested against benchmark engineered MBL (produced by transient expression) in multiple assays, including anti-Fc ELISA for productivity, mannan binding for potency, and HPLC-SEC and SDS-PAGE for purity and assembly. Once about 1 gm of engineered MBL is produced, stable clones producing the engineered MBL can be used to manufacture this opsonin.

Although MBL has a wide spectrum binding, there are a number of pathogenic microbes (e.g., encapsulated gram positive bacteria, such as S. aureus and S. pneumonia, as well E. fecaelis and H1N virus) that currently elude recognition by MBL. In order to achieve a generic pathogen isolating microfluidic device capability, knowledge of MBL's mannose binding site (Chang et al., J. Mol. Biol., 1994, 5: 241(1): 125-127) can be leveraged and mutagenesis can be used with directed evolution technologies to increase MBL's spectrum of pathogen binding. An opsonin display library with carbohydrate binding regions of MBL displayed on phage can be built, combined with many rounds of positive and negative screening in a short period of time using different surface targets from various pathogens that are not recognized by native MBL>Because the phage is expressed in bacteria, the Multiplexed Automated Genome Engineering (MAGE) technology recently developed by George Church at the Wyss Institute can be used to rapidly modify the sequence of the phage DNA encoding the MBL. MAGE utilizes an automated recombination-based genetic engineering approach to rapidly alter thousands of specific chromosomal sites in a living cell at high efficiency, providing the ability to generate up to 4.3 billion different genomic variants per day. This can allow creation of MBL opsonins that can be selectively induced to release bound pathogens so that opsonin-coated beads can be recycled back into the microfluidic device for repeated rounds of pathogen isolation. Selection techniques using panels of pathogenic microbes that are not recognized by natural MBL (or antigens from these pathogens expressed as Fc fusion proteins) can be used to identify modified versions of engineered MBL that bind to a broader spectrum of pathogens. One can screen for bound proteins using pull down assay with magnetically-tagged pathogens or toxins. In addition, the avidity of pathogen binding can be increased by fusing MBL to IgM rather than IgG, and these engineered ligands can be tested at different bead coating densities to optimize mutlivalency.

Nucleic acid based binding molecules include aptamers. As used herein, the term “aptamer” means a single-stranded, partially single-stranded, partially double-stranded or double-stranded nucleotide sequence capable of specifically recognizing a selected non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and normucleotide residues, groups or bridges. Methods for selecting aptamers for binding to a molecule are widely known in the art and easily accessible to one of ordinary skill in the art. The oligonucleotides including aptamers can be of any length, e.g., from about 1 nucleotide to about 100 nucleotides, from about 5 nucleotides to about 50 nucleotides, or from about 10 nucleotides to about 25 nucleotides. Generally, a longer oligonucleotide for hybridization to a nucleic acid scaffold can generate a stronger binding strength between the engineered microbe surface-binding domain and substrate.

In some embodiments of the aspects described herein, the binding molecules can be polyclonal and/or monoclonal antibodies and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Accordingly, as used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)]. Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

In some embodiments, the binding molecule can bind with a cell-surface marker or cell-surface molecule. In some further embodiments, the binding molecule binds with a cell-surface marker but does not cause initiation of downstream signaling event mediated by that cell-surface marker. Binding molecules specific for cell-surface molecules include, but are not limited to, antibodies or fragments thereof, natural or recombinant ligands, small molecules, nucleic acids and analogues thereof, intrabodies, aptamers, lectins, and other proteins or peptides.

As used herein, a “cell-surface marker” refers to any molecule that is present on the outer surface of a cell. Some molecules that are normally not found on the cell-surface can be engineered by recombinant techniques to be expressed on the surface of a cell. Many naturally occurring cell-surface markers present on mammalian cells are termed “CD” or “cluster of differentiation” molecules. Cell-surface markers often provide antigenic determinants to which antibodies can bind to.

Accordingly, as defined herein, a “binding molecule specific for a cell-surface marker” refers to any molecule that can selectively react with or bind to that cell-surface marker, but has little or no detectable reactivity to another cell-surface marker or antigen. Without wishing to be bound by theory, affinity molecules specific for cell-surface markers generally recognize unique structural features of the markers. In some embodiments of the aspects described herein, the preferred affinity molecules specific for cell-surface markers are polyclonal and/or monoclonal antibodies and antigen-binding derivatives or fragments thereof.

The binding molecule can be conjugated to the magnetic particle using any of a variety of methods known to those of skill in the art. The affinity molecule can be coupled or conjugated to the magnetic particles covalently or non-covalently. The covalent linkage between the affinity molecule and the magnetic particle can be mediated by a linker. The non-covalent linkage between the affinity molecule and the magnetic particle can be based on ionic interactions, van der Waals interactions, dipole-dipole interactions, hydrogen bonds, electrostatic interactions, and/or shape recognition interactions.

As used herein, the term “linker” means an organic moiety that connects two parts of a compound. Linkers typically comprise a direct bond or an atom such as oxygen or sulfur, a unit such as NH, C(O), C(O)NH, SO, SO₂, SO₂NH or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, C(O).

In some embodiments, the binding molecule is coupled to the magnetic particle by use of a coupling molecule pair. As used herein, the term “coupling molecule pair” refers to a pair of first and second molecules that specifically bind to each other. One member of the coupling pair is conjugated with the affinity molecule while the second member is conjugated with the magnetic particle. As used herein, the term “specific binding” refers to binding of the first member of the binding pair to the second member of the binding pair with greater affinity and specificity than to other molecules.

Exemplary binding pairs include any haptenic or antigenic compound in combination with a corresponding antibody or binding portion or fragment thereof (e.g., digoxigenin and anti-digoxigenin; mouse immunoglobulin and goat anti-mouse immunoglobulin) and nonimmunological binding pairs (e.g., biotin-avidin, biotin-streptavidin, hormone [e.g., thyroxine and cortisol-hormone binding protein, receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

One non-limiting example of using conjugation with a coupling molecule pair is the biotin-sandwich method. See, e.g., Davis et al., 103 PNAS 8155 (2006). The two molecules to be conjugated together are biotinylated and then conjugated together using tetravalent streptavidin. In addition, a peptide can be coupled to the 15-amino acid sequence of an acceptor peptide for biotinylation (referred to as AP; Chen et al., 2 Nat. Methods 99 (2005)). The acceptor peptide sequence allows site-specific biotinylation by the E. Coli enzyme biotin ligase (BirA; Id.). An engineered microbe surface-binding domain can be similarly biotinylated for conjugation with a solid substrate. Many commercial kits are also available for biotinylating proteins. Another example for conjugation to a solid surface would be to use PLP-mediated bioconjugation. See, e.g., Witus et al., 132 JACS 16812 (2010).

In some cases, the target component comprises one member of an affinity binding pair. In such cases, the second member of the binding pair can be conjugated to a magnetic particle as an affinity molecule.

In some embodiments, the magnetic particle is functionalized with two or more different affinity molecules. The two or more different affinity molecules can target the same target component or different target components. For example, a magnetic particle can be functionalized with antibodies and lectins to simultaneously target multiple surface antigens or cell-surface markers. In another example, a magnetic particle can be functionalized with antibodies that target surface antigens or cell-surface markers on different cells, or with lectins, such as mannose-binding lectin, that recognizes surface markers on a wide variety of pathogens.

In some embodiments, the binding/affinity molecule is a ligand that binds to a receptor on the surface of a target cell. Such a ligand can be a naturally occurring molecule, a fragment thereof or a synthetic molecule or fragment thereof. In some embodiments, the ligand is non-natural molecule selected for binding with a target cell. High throughput methods for selecting non-natural cell binding ligands are known in the art and easily available to one of skill in the art. See for example, Anderson, et al., Biomaterial microarrays: rapid, microscale screening of polymer-cell interaction. Biomaterials (2005) 26:4892-4897; Anderson, et al., Nanoliter-scale synthesis of arrayed biomaterials and application to human embryonic stem cells. Nature Biotechnology (2004) 22:863-866; Orner, et al., Arrays for the combinatorial exploration of cell adhesion. Journal of the American Chemical Society (2004) 126:10808-10809; Falsey, et al., Peptide and small molecule microarray for high throughput cell adhesion and functional assays. Bioconjugate Chemistry (2001) 12:346-353; Liu, et al., Biomacromolecules (2001) 2(2): 362-368; and Taurniare, et al., Chem. Comm. (2006): 2118-2120.

In some embodiments, the binding molecule and/or the magnetic particles can be conjugated with a label, such as a fluorescent label or a biotin label. When conjugated with a label, the binding molecule and the magnetic particle are referred to as “labeled binding molecule” and “labeled magnetic particles” respectively. In some embodiments, the binding molecule and the magnetic particles are both independently conjugated with a label, such as a fluorescent label or a biotin label. Without wishing to be bound by theory, such labeling allows one to easily track the efficiency and/or effectiveness of methods to selectively bind the target component in a source fluid. For example, a multi-fluorescence labeling can be used to distinguish between free magnetic particles, free target components and magnetic particle-target component complexes.

As used herein, the term “label” refers to a composition capable of producing a detectable signal indicative of the presence of a target. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means that can be used in the methods and devices described herein. For example, binding molecules and/or magnetic particles can also be labeled with a detectable tag, such as c-Myc, HA, VSV-G, HSV, FLAG, V5, or HIS, which can be detected using an antibody specific to the label, for example, an anti-c-Myc antibody.

Exemplary fluorescent labels include, but are not limited to, Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), Fluor X, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

The degree of magnetic particle binding to a target component is such that the bound target component will move when a magnetic field is applied. It is to be understood that binding of magnetic particle with the target component is mediated through affinity molecules, i.e., the affinity molecule on the surface of the magnetic particle that binds to the target component. Binding of magnetic particles to target components can be determined using methods or assays known to one of skill in the art, such as ligand binding kinetic assays and saturation assays. For example, binding kinetics of a target component and the magnetic particle can be examined under batch conditions to optimize the degree of binding. In another example, the amount of magnetic particles needed to bind a target component can be ascertained by varying the ratio of magnetic particles to target component under batch conditions. The binding efficiency can follow any kinetic relationship, such as a first-order relationship. In some embodiments, binding efficiency follows a Langmuir adsorption model.

The separation efficiency of a microfluidic device described herein can be determined using methods known in the art and easily adaptable for microfluidic devices. For example, magnetic particle conjugated with an affinity molecule and the target components are pre-incubated in the appropriate medium to allow maximum binding before resuspending in a source fluid. The effects of varying electromagnet current on separation efficiency can be analyzed using, for example, target component-magnetic particle complexes suspended in PBS. To test how the viscosity of the collection fluid affected its hydrodynamic interaction with a biological fluid, such as blood, medical grade dextran (40 kDa, Sigma) can be used to vary the viscosity. For example, dextran can be dissolved in PBS at 5, 10 and 20% to produce solutions with viscosities of 2, 3, 11 centipoise at room temperature. Samples can be collected from source inlet, source outlet, and source channels and analyzed by flow cytometry to assess the separation efficiency of magnetic particles and particle bound target components. Efficiency can be calculated as: Efficiency=1−X_{source-out}/X_{source-in}. Source fluid loss can be quantified using an appropriate marker in the source fluid. For example, blood loss can be quantified by measuring the OD600 of red blood cells (Loss=OD_{collection-out}/OD_{source-out}).

The optimal time for binding of magnetic particles to target component can vary depending on the particulars of the device or methods being employed. The optimal mixing and/or incubation time for binding of magnetic particles to a target component can be determined using kinetic assays well known to one of skill in the art. For example, kinetic assays can be performed under conditions that mimic the particulars of the device or methods to be employed, such as volumes, concentrations, how and where the mixing is to be performed, and the like. The rate of binding of magnetic particles to target components can be increased by carrying out mixing within separate microfluidic mixing channels.

As used herein, the term “target component” refers to any molecule, cell or particulate that is to be filtered or separated from a source fluid. Representative examples of target cellular components include, but are not limited to, mammalian cells, viruses, bacteria, fungi, yeast, protozoan, microbes, parasites, and the like. Representative examples of target molecules include, but are not limited to, hormones, cytokines, proteins, peptides, prions, lectins, oligonucleotides, contaminating molecules and particles, molecular and chemical toxins, exosomes, and the like. The target components also include contaminants found in non-biological fluids, such as pathogens or lead in water or in petroleum products. Parasites include organisms within the phyla Protozoa, Platyhelminthes, Aschelminithes, Acanthocephala, and Arthropoda.

As used herein, the term “molecular toxin” refers to a compound produced by an organism which causes or initiates the development of a noxious, poisonous or deleterious effect in a host presented with the toxin. Such deleterious conditions may include fever, nausea, diarrhea, weight loss, neurologic disorders, renal disorders, hemorrhage, and the like. Toxins include, but are not limited to, bacterial toxins, such as cholera toxin, heat-liable and heat-stable toxins of E. coli, toxins A and B of Clostridium difficile, aerolysins, hemolysins, and the like; toxins produced by protozoa, such as Giardia; toxins produced by fungi; and the like. Included within this term are exotoxins, i.e., toxins secreted by an organism as an extracellular product, and enterotoxins, i.e., toxins present in the gut of an organism.

In some embodiments, the target component is a bioparticle/pathogen selected from the group consisting of living or dead cells (prokaryotic and eukaryotic, including mammalian), viruses, bacteria, fungi, yeast, protozoan, microbes, parasites, and the like. As used herein, a pathogen is any disease causing organism or microorganism.

Exemplary mammalian cells include, but are not limited to, stem cells, cancer cells, progenitor cells, immune cells, blood cells, fetal cells, and the like.

Exemplary fungi and yeast include, but are not limited to, Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii (or Pneumocystis carinii), Stachybotrys chartarum, and any combination thereof.

Exemplary bacteria include, but are not limited to: anthrax, campylobacter, cholera, diphtheria, enterotoxigenic E. coli, giardia, gonococcus, Helicobacter pylori, Hemophilus influenza B, Hemophilus influenza non-typable, meningococcus, pertussis, pneumococcus, salmonella, shigella, Streptococcus B, group A Streptococcus, tetanus, Vibrio cholerae, yersinia, Staphylococcus, Pseudomonas species, Clostridia species, Myocobacterium tuberculosis, Mycobacterium leprae, Listeria monocytogenes, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Treponema pallidum, Haemophilus influenzae, Treponema pallidum, Klebsiella pneumoniae, Pseudomonas aeruginosa, Cryptosporidium parvum, Streptococcus pneumoniae, Bordetella pertussis, Neisseria meningitides, and any combination thereof.

Exemplary parasites include, but are not limited to: Entamoeba histolytica; Plasmodium species, Leishmania species, Toxoplasmosis, Helminths, and any combination thereof.

Exemplary viruses include, but are not limited to, HIV-1, HIV-2, hepatitis viruses (including hepatitis B and C), Ebola virus, West Nile virus, and herpes virus such as HSV-2, adenovirus, dengue serotypes 1 to 4, ebola, enterovirus, herpes simplex virus 1 or 2, influenza, Japanese equine encephalitis, Norwalk, papilloma virus, parvovirus B19, rubella, rubeola, vaccinia, varicella, Cytomegalovirus, Epstein-Barr virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, poliovirus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Human T-cell Leukemia virus type-1, Hantavirus, Rubella virus, Simian Immunodeficiency viruses, and any combination thereof.

Exemplary contaminants found in non-biological fluids can include, but are not limited to microorganisms (e.g., Cryptosporidium, Giardia lamblia, bacteria, Legionella, Coliforms, viruses, fungi), bromates, chlorites, haloactic acids, trihalomethanes, chloramines, chlorine, chlorine dioxide, antimony, arsenic, mercury (inorganic), nitrates, nitrites, selenium, thallium, Acrylamide, Alachlor, Atrazine, Benzene, Benzo(a)pyrene (PAHs), Carbofuran, Carbon, etrachloride, Chlordane, Chlorobenzene, 2,4-D, Dalapon, 1,2-Dibromo-3-chloropropane (DBCP), o-Dichlorobenzene, p-Dichlorobenzene, 1,2-Dichloroethane, 1,1-Dichloroethylene, cis-1,2-Dichloroethylene, trans-1,2-Dichloroethylene, Dichloromethane, 1,2-Dichloropropane, Di(2-ethylhexyl) adipate, Di(2-ethylhexyl) phthalate, Dinoseb, Dioxin (2,3,7,8-TCDD), Diquat, Endothall, Endrin, Epichlorohydrin, Ethylbenzene, Ethylene dibromide, Glyphosate, Heptachlor, Heptachlor epoxide, Hexachlorobenzene, Hexachlorocyclopentadiene, Lead, Lindane, Methoxychlor, Oxamyl (Vydate), Polychlorinated, biphenyls (PCBs), Pentachlorophenol, Picloram, Simazine, Styrene, Tetrachloroethylene, Toluene, Toxaphene, 2,4,5-TP (Silvex), 1,2,4-Trichlorobenzene, 1,1,1-Trichloroethane, 1,1,2-Trichloroethane, Trichloroethylene, Vinyl chloride, and Xylenes.

### Exemplary Uses for the Devices

The devices, systems, and methods described herein provide novel advantages for a variety of application including, but not limited to, therapeutic application (e.g., biofiltrations, toxin clearance, pathogen clearance, removal of cytokines or immune modulators), filtrations, enrichment, purifications, diagnostics, and the like.

In some embodiments, the devices, systems, and methods described herein are used to selectively separate target components from source fluids. For a non-limiting example, the devices, systems, and methods provided herein can be used for separating cells, bioparticles, pathogens, molecules and/or toxins from a biological fluid in treating a subject in need thereof.

Separated target components can be utilized for any purpose including, but not limited to, diagnosis, culture, sensitivity testing, drug resistance testing, pathogen typing or sub-typing, PCR, NMR, mass spectroscopy, IR spectroscopy, immunostaining, and immunoassaying.

Identification and typing of pathogens is critical in the clinical management of infectious diseases. Precise identity of a microbe is used not only to differentiate a disease state from a healthy state, but is also fundamental to determining whether and which antibiotics or other antimicrobial therapies are most suitable for treatment. Thus, pathogens separated from a subject's blood can be used for pathogen typing and sub-typing. Methods of pathogen typing are well known in the art and include using a variety of phenotypic features such as growth characteristics; color; cell or colony morphology; antibiotic susceptibility; staining; smell; and reactivity with specific antibodies, and molecular methods such as genotyping by hybridization of specific nucleic acid probes to the DNA or RNA; genome sequencing; RFLP; and PCR fingerprinting.

In PCR finger printing, the size of a fragment generated by PCR is used as an identifier. In this type of assay, the primers are targeted to regions containing variable numbers of tandem repeated sequences (referred to as VNTRs an eukaryotes). The number of repeats, and thus the length of the PCR amplicon, can be characteristic of a given pathogen, and co-amplification of several of these loci in a single reaction can create specific and reproducible fingerprints, allowing discrimination between closely related species. In cases where organisms are very closely related, the target of the amplification may not display a size difference, and the amplified segment must be further probed to achieve more precise identification. This can be accomplished by using the interior of the PCR fragment as a template for a sequence-specific ligation event.

The methods, systems, and devices described herein can also be used to determine if there are different sub-populations of a pathogen or a combination of different pathogens present in an infected subject. The ability to quickly determine subtypes of pathogens can allow comparisons of the clinical outcomes from infection by the different pathogen subtypes, and from infection by multiple types in a single individual. In many cases, a pathogen subtype has been associated with differential efficacy of treatment with a specific drug. For example, HCV type has been associated with differential efficacy of treatment with interferon. Pre-screening of infected individuals for the pathogen subtype type can allow the clinician to make a more accurate diagnosis, and to avoid costly but fruitless drug treatment.

As used herein, removing or separating target components means that the amount of the target component is reduced by at least 5%, 10%, 20%, 30%, 40%, 50%, 60%, 70%, 75%, 80%, 85%, 90%, 95%, 100% (completely reduction) in the source fluid.

Pathogen Clearance from Blood

In some embodiments, the devices, systems, and methods provided herein are used to remove sepsis related target components from the blood of a subject in need thereof. As used herein, sepsis related target components refer to any molecule or bioparticle that can contribute to development of sepsis in a subject.

As used herein, “sepsis” refers to a body or subject's response to a systemic microbial infection. Sepsis is the leading cause of death of immunocompromised patients, and is responsible for over 200,000 deaths per year in the United States. The onset of sepsis occurs when rapidly growing infectious agents saturate the blood and overcome a subject's immunological clearance mechanisms. Most existing therapies are ineffective, and subjects can die because of clot formation, hypoperfusion, shock, and multiple organ failure.

In some embodiments, the devices, systems, and methods provided herein are used to in combination with conventional therapies for treating a subject in need thereof. For example, the devices, systems, and methods provided herein are used in conjunction with conventional therapies for sepsis treatment, such as fungicides. In another example, the devices, systems, and methods described herein are used for treating a subject having a cancer. The method comprising removing cancer cells from a biological fluid obtained from the subject, and providing an additional treatment including, but not limited to, chemotherapy, radiation therapy, steroids, bone marrow transplants, stem cell transplants, growth factor administration, ATRA (all-trans-retinoic acid) administration, histamine dihydrochloride (Ceplene) administration, interleukin-2 (Proleukin) administration, gemtuzumab ozogamicin (Mylotarg) administration, clofarabine administration, farnesyl transferase inhibitor administration, decitabine administration, inhibitor of MDR1 (multidrug-resistance protein) administration, arsenic trioxide administration, rituximab administration, cytarabine (ara-C) administration, anthracycline administration (such as daunorubicin or idarubicin), imatinib administration, dasatanib administration, nilotinib administration, purine analogue (such as fludarabine) administration, alemtuzumab (anti-CD52) administration, (fludarabine with cyclophosphamide), fludarabine administration, cyclophosphamide administration, doxorubicin administration, vincristine administration, prednisolone administration, lenalidomide administration, flavopiridol administration, or any combination therein. In some embodiments, the devices, systems, and methods provided herein are used for treating a subject in need thereof without providing any other therapy to the subject. For example, the devices, systems, and methods provided herein are used for sepsis treatment, pathogen and/or toxin clearance from biological fluids, of a subject in need thereof.

In some embodiments, the devices, systems, and methods described herein are used to purify or enrich a target component from a source fluid. For example, the devices, systems, and methods described herein can be used to purify products of chemical reactions or molecules being produced in a cell culture.

Inventors have already carried out in vivo testing of the microfluidic device for pathogen clearance. In vivo testing of the microfluidic device for pathogen clearance was tested in rabbits injected intravenously with fungal pathogens. The microfluidic device was well tolerated by rabbits even after 30 minutes of continuous blood perfusion (12 mL/hr) through the microfluidic system. In order to reduce the healthy spleen from filtering out majority of the microbes mintues after i.v. injection, a more physiologically relevant sepsis animal model can be used. For example, a rat intra-abdominal sepsis model (Weinstein et al., Infect. Immun., 1974, 10(6): 1250-1255) can be established to determine or demonstrate the efficacy of microfluidic device using broad spectrum opsonins. This model was developed by Dr. Andrew Onderdonk (Onderdonk et al., Infect. Immun., 1974, 10(6): 1255-1259) and has been used in the approval of all major antibiotics since 1979.

Disseminated septicemia is produces by implanting an inoculum of cecal conents from one rat, or a known culture of bacterial or fungal microbes, into the peritoneal cavity of another. The cecal inoculum is complex and contains a mixture of facultative organisms (e.g., E. coli, Enteroccoccus, Steptococcus, and Staphyloccocus), as well as obligate anaerobes (e.g., Bacteroides, Prevotella, Clostridium, and Fusobacterium). The infectious process that occurs in rats is similar to that which would occur in humans following trauma to the large bowel, such as gunshot wounds, knife wounds, bowel rupture following trauma, and accidental peritoneal soilage during colon surgery.

Testing of the microfluidic device can be carried out in the rat model with MBL coated magnetic beads. Pathogen numbers can be quantitated in blood samples taken from animals over time after implantation of the infectious pathogens, and blood cleansing studies can be initiated 24 hours after microbe can be detectable in these samples. Catheters can be surgically placed into the two femoral veins of the rats, and hepranized blood can be reirculated through the biomimetic spleen device using a blood infusion pump (flow rate<100 mL/hr); compatible blood from healthy donor rats can be used to prime the circuit. The blood cleansing efficiency can be determined after passing blood for 3 hours through the device (which is enough time for entire blood volume of the rat to pass multiple times through the system), and also the animal survival can be measured over the following 5 days.

Accordingly, provided herein is blood cleansing device that is robust, portable, capable of handling continuous flow at high rates, and easily inserted within the peripheral vessels of a sick subject, patient, or solider to remove blood-borne pathogens, without having to first identify the source of infection.

Isolation and Enrichment of Rare Populations of Cells from Source Fluids

In some aspects of the invention, the methods, devices, and systems described herein can be used for isolating and enriching for rare cell populations, such as stem cells, progenitor cells, cancer cells, or fetal cells from source fluids. Because the entire blood volume of a patient can be circulated through the device, low frequency populations can be identified using this method. Such populations of cells may represent a small fraction of cells present in a source fluid, and may be otherwise difficult to isolate or enrich for.

A source fluid from which rare populations of cells can be isolated from or enriched for can be any fluid sample in which such cells may be present. In some embodiments, the source fluid is a biological sample that is found naturally in the fluid form, such as whole blood, plasma, serum, amniotic fluid, cord blood, lymph fluid, cerebrospinal fluid, urine, sputum, pleural fluid, tears, breast milk, nipple aspirates, and saliva. In other embodiments, the biofluid sample is a fluid sample prepared from a solid or semi-solid tissue, organ, or other biological sample from which rare cell populations may be isolated or enriched for. In such embodiments, single-cell populations may be prepared from a tissue or organ, and resuspended in a buffer, such as saline solutions containing serum, for use in the methods and devices described herein. Such single-cell suspensions may be prepared using any method known to one of skill in the art, such as manual methods using slides, enzyme treatment, or tissue dissociators. Tissues and organs from which single-cell suspensions may be prepared for use in the methods and devices described herein, include, but are not limited to, bone marrow, thymus, stool, skin sections, spleen tissue, pancreatic tissue, cardiac tissue, lung tissue, adipose tissue, connective tissue, sub-epithelial tissue, epithelial tissue, liver tissue, kidney tissue, uterine tissue, respiratory tissues, gastrointestinal tissue, genitourinary tract tissue and cancerous tissues.

In one or more embodiments of the aspects, rare populations of cells, such as stem cells, can be identified for isolation and enrichment using the methods, devices, and systems described herein by one or more markers, such as cell-surface markers, specific for the rare cell population. Accordingly, in such embodiments, magnetic particles bound to or conjugated to a binding molecule specific for one or more of the markers present on or in the rare cell population can be used. In some embodiments, the affinity molecule is an antibody or antigen-binding fragment specific for a marker. In some embodiments, one or more affinity molecules specific for one or more markers found on or in a rare cell population are conjugated to magnetic particles. For example, one magnetic particle can be conjugated to multiple different affinity molecules, where each affinity molecule is specific for a different marker associated with the rare cell population. In another example, a combination of magnetic particles is used, where each magnetic particle is conjugated or bound to affinity molecules specific for a single cell marker, and a combination of such particles is used to isolate or enrich for a rare cell population. In one or more embodiments, the rare cell population is a stem cell or progenitor cell population.

Exemplary cell markers can include, but are not limited to, one or more of the following markers: c-Myc, CCR4, CD15 (SSEA-1, Lewis X), CD24, CD29 (Integrin β1), CD30, CD49f (Integrin α6), CD9, CDw338 (ABCG2), E-Cadherin, Nanog, Oct3/4, Smad2/3, So72, SSEA-3, SSEA-4, STAT3 (pS727), STAT3 (pY705), STAT3, TRA-1-60, TRA-1-81, CD117 (SCF R, c-kit), CD15 (SSEA-1, Lewis X), VASA (DDX4), CD72, Cytokeratin 7, Trop-2, GFAP, S100B, Nestin, Notch1, CD271 (p75, NGFR/NTR), CD49d (Integrin α4), CD57 (FINK-1), MASH1, Neurogenin 3, CD146 (MCAM, MUC18), CD15s (Sialyl Lewis x), CD184 (CXCR4), CD54 (ICAM-1), CD81 (TAPA-1), CD95 (Fas/APO-1), CDw338 (ABCG2), Ki-67, Noggin, So71, So72, Vimentin, α-Synuclein (pY125), α-Synuclein, CD112, CD56 (NCAM), CD90 (Thy-1), CD90.1 (Thy-1.1), CD90.2 (Thy-1.2), ChAT, Contactin, Doublecortin, GABA A Receptor, Gad65, GAP-43 (Neuromodulin), GluR delta 2, GluR2, GluR5/6/7, Glutamine Synthetase, Jagged1, MAP2 (a+b), MAP2B, mGluR1 alpha, mGluR1, N-Cadherin, Neurofilament NF—H, Neurofilament NF-M, Neuropilin-2, Nicastrin, P-glycoprotein, p150 Glued, Pax-5, PSD-95, Serotonin Receptor 5-HT 2AR, Serotonin Receptor 5-HT 2BR, SMN, Synapsin I, Synaptophysin, Synaptotagmin, Syntaxin, Tau, TrkB, Tubby, Tyrosine Hydroxylase, Vimentin, CD140a (PDGFR α), CD44, CD44H (Pgp-1, H-CAM), CRABP2, Fibronectin, Sca-1 (Ly6A/E), β-Catenin, GATA4, HNF-1β (TCF-2), N-Cadherin, HNF-1α, Tat-SF1, CD49f (Integrin α6), Gad67, Neuropilin-2, CD72, CD31 (PECAM1), CD325 (M-Cadherin), CD34 (Mucosialin, gp 105-120), NF-YA, CD102, CD105 (Endoglin), CD106 (VCAM-1), CD109, CD112, CD116 (GM-CSF Receptor), CD117 (SCF R, c-kit), CD120a (TNF Receptor Type I), CD120b (TNF Receptor Type II), CD121a (IL-1 Receptor, Type I/p80), CD124 (IL-4 Receptor α), CD141 (Thrombomodulin), CD144 (VE-cadherin), CD146 (MCAM, MUC18), CD147 (Neurothelin), CD14, CD151, CD152 (CTLA-4), CD157, CD166 (ALCAM), CD18 (Integrin β2 chain, CR3/CR4), CD192 (CCR2), CD201 (EPCR), CD202b (TIE2) (pY1102), CD202b (TIE2) (pY992), CD202b (TIE2), CD209, CD209a (CIRE, DC-SIGN), CD252 (OX-40 Ligand), CD253 (TRAIL), CD262 (TRAIL-R2, DR5), CD325 (M-Cadherin), CD36, CD45 (Leukocyte Common Antigen, Ly-5), CD45R (B220), CD49d (Integrin α4), CD49e (Integrin α5), CD49f (Integrin α6), CD54 (ICAM-1), CD56 (NCAM), CD62E (E-Selectin), CD62L (L-Selectin), CD62P(P-Selectin), CDw93 (C1qRp), Flk-1 (KDR, VEGF-R2, Ly-73), HIF-1α, IP-10, α-Actinin, Annexin VI, Caveolin-2, Caveolin-3, CD66, CD66c, Connexin-43, Desmin, Myogenin, N-Cadherin, CD325 (E-Cadherin), CD10, CD124 (IL-4 Receptor α), CD127 (IL-7 Receptor α), CD38, HLA-DR, Terminal Transferase (TdT), CD41, CD61 (Integrin β3), CD11c, CD13, CD114 (G-CSF Receptor), CD71 (Transferrin Receptor), PU.1, TER-119/Erythroid cells (Ly-76), CaM Kinase IV, CD164, CD201 (EPCR), CDw338 (ABCG2), CDw93 (C1qRp), MRP1, Notch1, P-glycoprotein, WASP (Wiskott-Aldrich Syndrome Protein), Acrp30 (Adiponectin), CD151, β-Enolase (ENO-3), Actin, CD146 (MCAM, MUC18), MyoD, IGFBP-3, CD271 (p75, NGFR/NTR), CD73 (Ecto-5′-nucleotidase), and TAZ.

As used herein, the terms “isolate” and “methods of isolation,” refers to a process whereby a target component is removed from a source fluid. In reference to isolation of cells, the terms “isolate” and “methods of isolation,” refers to a process whereby a cell or population of cells is removed from a subject or fluid sample in which it was originally found, or a descendant of such a cell or cells. The term “isolated population” with respect to an isolated population of cells, as used herein, refers to a population of cells that has been removed and separated from a source fluid, or a mixed or heterogeneous population of cells found in such a sample. Such a mixed population includes, for example, a population of peripheral blood mononuclear cells obtained from isolated blood, or a cell suspension of a tissue sample, such as a single-cell suspension prepared from the spleen. In one or more embodiments, an isolated population is a substantially pure population of cells as compared to the heterogeneous population from which the cells were isolated or enriched from. In one or more embodiments of this aspect and all aspects described herein, the isolated population is an isolated population of progenitor cells. In one or more embodiments, an isolated cell or cell population, such as a population of progenitor cells, is further cultured in vitro, e.g., in the presence of growth factors or cytokines, to further expand the number of cells in the isolated cell population or substantially pure cell population. Such culture can be performed using any method known to one of skill in the art. In one or more embodiments, the isolated or substantially pure progenitor cell populations obtained by the methods disclosed herein are later introduced into a second subject, or re-introduced into the subject from which the cell population was originally isolated (e.g., allogenic transplantation).

As used herein, the term “substantially pure,” with respect to a particular cell population, refers to a population of cells that is at least about 75%, at least about 80%, at least about 85%, at least about 90%, at least about 95%, at least about 98%, or at least about 99% pure, with respect to the cells making up a total cell population. In other words, the terms “substantially pure” or “essentially purified”, with regard to a population of progenitor cells isolated using the methods as disclosed herein, refers to a population of progenitor cells that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 4%, fewer than about 3%, fewer than about 2%, fewer than about 1%, or less than 1%, of cells that are not progenitor cells as defined by the terms herein.

In some embodiments, rare populations of cells are enriched for using the methods, systems, and devices described herein. The terms “enriching” or “enriched” are used interchangeably herein and mean that the yield (fraction) of cells of one type, such as progenitor cells, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biofluid sample, such as a culture or human whole blood.

Removal of Cancer Cells from Source Fluids

The methods, systems, and devices described herein can also provide novel advantages for use in therapies for cancer treatment, such as removal of cancer cells present in source fluids obtained from a patient or subject at risk for or having a cancer, such as hematological malignancies or metastatic cells from other organ sites. In one or more embodiments, the cancer cell is an ALL, B-CLL, CML, AML cancer cell, or a cancer cells from the breast, lung, kidney, brain, spinal cord, liver, spleen, blood, bronchi, central nervous system, cervix, colon, rectum and appendix, large intestine, small intestine, bladder, testicles, ovaries, pelvis, lymph nodes, esophagus, uterus, bile ducts, pancreas, gall bladder, uvea, retina, upper aerodigestive tract (e.g., lip, oral cavity (mouth), nasal cavity, paranasal sinuses, pharynx, and larynx), ovaries, parathyroid glands, pineal glands, pituitary gland, prostate, connective tissue, skeletal muscle, salivary gland, thyroid gland, thymus gland, urethra, or vulva.

As used herein, “hematological malignancies” refers to those types of cancer that affect blood, bone marrow, and lymph nodes. As the three are intimately connected through the immune system, a disease affecting one of the three will often affect the others as well: although lymphoma is technically a disease of the lymph nodes, it often spreads to the bone marrow, affecting the blood and occasionally produces a paraprotein.

Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are conditions that arise from the lymphoid line, while acute and chronic myelogenous leukemia, myelodysplastic syndromes and myeloproliferative diseases involve cancer cells that are myeloid in origin.

In some embodiments of the aspects, subject having or at risk for a cancer, such as ALL, B-CLL, CML or AML, is treated using the methods, devices, and systems described herein. In such embodiments, the methods, devices, and systems described herein are used to remove cancer cells from a source fluid obtained from a subject having or at risk for a cancer. some embodiments, the source fluid is a biological fluid such as blood or bone marrow obtained from the subject.

In some embodiments, binding molecules specific for one or more markers, such as cell-surface markers, specific for the cancer cell population are used to remove cancer cells from a source fluid obtained from a subject. Accordingly, in such embodiments, magnetic particles bound to or conjugated to binding molecules specific for one or more of the markers present on or in the cancer cell population can be used. In some embodiments, the binding molecule is an antibody or antigen-binding fragment specific for a marker present on or in the cancer cell population. For example, in some embodiments, a monoclonal antibody specific for a B cell light chain present only on CLL cells can be bound to or conjugated to magnetic particles, and such conjugated magnetic particles can be contacted with a fluid sample from a subject having CLL to remove CLL cells, using the methods, devices, and systems described herein.

In some embodiments, one or more binding molecules specific for one or more markers found on or in a cancer cell population are conjugated to magnetic particles. For example, one magnetic particle can be conjugated to multiple different affinity molecules, where each binding molecule is specific for a different marker associated with the cancer cell population. In another example, a combination of magnetic particles is used, where each magnetic particle is conjugated or bound to one type of binding molecule, such as an antibody specific for a cancer cell surface marker, and a combination of such particles is used to isolate or enrich for the cancer cell population.

Exemplary cancer markers include, but are not limited to, CD19, CD20, CD22, CD33, CD52, monotypic surface IgM, CD10, Bcl-6, CD79a, CD5, CD23, and Terminal deoxytransferase (TdT). Any additional markers that are identified as being unique to or increased upon cancer cells, such as leukemias, are also included within the scope of the methods, devices, and systems described herein.

Other cancer antigens useful within the scope of the methods, devices, and systems described herein, include, for example PSA, Her-2, Mic-1, CEA, PSMA, mini-MUC, MUC-1, HER2 receptor, mammoglobulin, labyrinthine, SCP-1, NY-ESO-1, SSX-2, N-terminal blocked soluble cytokeratin, 43 kD human cancer antigens, PRAT, TUAN, Lb antigen, carcinoembryonic antigen, polyadenylate polymerase, p53, mdm-2, p21, CA15-3, oncoprotein 18/stathmin, and human glandular kallikrein), melanoma antigens, and the like.

In other embodiments of the aspects described herein, the methods and systems comprise removing target cancer cells from a source fluid obtained from a subject having or at risk for cancer and further comprise subjecting the removed cancer cells to genetic analyses to identify the cause or nature of the cancer. Such identification can enable enhanced treatment modalities and efficacy. Without wishing to be bound by theory, this can further allow the methods, devices and systems described herein to be used in personalized medicine treatments. For example, such genetic analyses on the removed cells can be used to identify which of the causal chromosomal translocation events involved in AML predisposition is causing a subject's AML, such as identifying that the translocation is occurring between chromosome 10 and 11.

As used herein, “cancer” refers to any of various malignant neoplasms characterized by the proliferation of neoplastic cells that tend to invade surrounding tissue and metastasize to new body sites and also refers to the pathological condition characterized by such malignant neoplastic growths. The blood vessels provide conduits to metastasize and spread elsewhere in the body. Upon arrival at the metastatic site, the cancer cells then work on establishing a new blood supply network. Encompassed in the methods disclosed herein are subjects that are treated for cancer, including but not limited to all types of carcinomas and sarcomas, such as those found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. The types of carcinomas include papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

The methods, devices and systems described herein are also useful in determining patient specific and general response of cancer patients to therapies (radiation or chemical). For example, circulating tumor cells from a subject can be isolated and analyzed before and after onset of a treatment regime. The methods, devices and systems described herein can also be used to determine cancer staging and/or early diagnosis of malignancy. For example, the magnetic particles can be tagged with a label for easy detection of free and cell bound particles. Separated cells can also analyzed for stage specific markers. The stage of a cancer is a descriptor (usually numbers Ito IV) of how much the cancer has spread. The stage often takes into account the size of a tumor, how deeply it has penetrated, whether it has invaded adjacent organs, how many lymph nodes it has metastasized to (if any), and whether it has spread to distant organs. Staging of cancer is important because the stage at diagnosis is the most powerful predictor of survival, and treatments are often changed based on the stage. Correct staging is critical because treatment is directly related to disease stage. Incorrect staging can lead to improper treatment, and material diminution of patient survivability. Oversight of one cell can mean mistagging and lead to serious, unexpected spread of cancer.

As used herein, the terms “treat” or “treatment” or “treating” refer to both therapeutic treatment and prophylactic or preventative measures, wherein the object is to prevent or slow the development of the disease. Without wishing to be limited by examples, if the disease is cancer, the slowing of the development of a tumor, the spread of cancer, or reducing at least one effect or symptom of a condition, disease or disorder associated with inappropriate proliferation or a cell mass, for example cancer would be considered a treatment. Treatment is generally “effective” if one or more symptoms or clinical markers are reduced as that term is defined herein.

Alternatively, treatment is “effective” if the progression of a disease is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer, as well as those likely to develop secondary tumors due to metastasis.

In some aspects, the methods, devices, and systems described herein can be used for analysis and for detecting the presence of target components in a source fluid. After separation form the source fluid, the target component can be analyzed using any method known in the art for detection of such a target component. For example, the target component can be tagged with a label such as dyes, antibodies, molecules which bind with the target component and easily detectable, or molecules which bind with the target component and are conjugated with a label. Alternatively, other methods such as optical techniques, e.g., microscopy, phase contrast imaging, etc. can be employed for detection of target components.

The collection fluid can be analyzed while the collection fluid is still in the collection microchannel or a portion of the collection fluid removed and the removed portion analyzed for presence of the target component. In some embodiments, magnetic particles from the collection fluid can be separated from the collection fluid and analyzed for presence of bound target components. In some embodiments, the outlet port of the collection channel can be connected to an inline or on-chip diagnostic device, used to analyze the target components. In this embodiment, the inline or on-chip diagnostic device can use magnetic field gradients to control the movement of the magnetically bound target components in order to subject them to inline analysis and testing and, for example, to provide detection of detection of low concentrations of pathogens in relatively small volumes of biofluids. For example, magnetic field gradients can be used to separate or isolate the magnetically bound target components from the collection fluid and then analyzed using one or more of dyes, antibodies, non-labeled optical or solid-state detection techniques.

Using an embodiment of the microfluidic device, comprising a central body fabricated from aluminum, inventors were able to isolate 1 μm magnetic bead bound C. albicans from blood with ˜90% isolation efficiency at 418 mL/h. Additionally, using two microfluidic devices in parallel, inventors were able to isolate 1 μm WT-MBL magnetic bead bound C. albicans from blood with over 85% isolation efficiency at 418 mL/h.

In one or more embodiments of the aspects described herein, a multiplexed device of the present invention was capable of over 85% cleansing of living fungal pathogens from a whole blood without inducing blood coagulation or causing significant loss of other blood cellular or molecular components. In some such embodiments, whole blood can flow at a rate of 836 mL. The results clearly demonstrate that the novel multiplexed microfluidic-micromagnetic cell separation designs described herein provide much higher volume throughput while maintaining target component separation efficiencies, and thus, confirm their value for clinical applications such as blood cleansing.

Innovations of the present design over previous designs for microfluidic-micromagnetic cell separators include that it uses neither (a) a second continually flowing stream of collection fluid (e.g., saline), nor (b) maintenance of a stable boundary between two laminar flow streams (which are central elements in the microfluidic devices described previously in US 2009-0078614 and US 2009-0220932) to remove particles. Thus, the present system is improved by its simplicity and robustness; blood also cannot be lost or diluted due an imbalance of hydrodynamics between blood and saline solutions. This biomimetic design emulates the sinus of the spleen where blood flow rate is relatively slow and episodic, and opsonized pathogens are retained. Saline in the collection channels is then used to periodically flush out the “sinus”, and this emulates the percolating flow of waste and lymph fluids through the lymphoid follicles.

### Fluid Cleaning

FIG. 20 shows a flow chart of a method for processing a fluid to remove target components bound to magnetic beads using a microfluidic device described herein. As shown in FIG. 20, at 2002, the collection fluid can be pumped into the collection channels and fill some or all of the transfer channels and the source channels. At 2004, the source fluid can be combined, such as by mixing, with the magnetic beads. The magnetic bead can be include an affinity coating that enables target components in the source fluid to bind to the magnetic beads. At 2006, the magnetic field gradient can be applied to the source channel, such as by applying power to an electromagnet or positioning permanent magnets at a predefined location with respect to the source channel. At 2008, the source fluid is pumped into and through the source channel, exposing the magnetic beads (and any target components bound thereto) to the magnet field gradient. At 2010, the magnetic bead and target components migrate through the transfer channels to the collection channels. At 2012, the system checks to determine whether a defined amount of magnetic beads have accumulated in the collection channel and the collection channel needs to be flushed. This can be after a predefined volume of source fluid flow or after a predefined period of time or based on a signal from a sensor, collection fluid can be allowed to flow into the collection channel, flushing the collection channels and magnetic beads out of the collection channels. During the flushing process, the source fluid flow can be reduced or stopped for the duration of the flushing process. If enough magnetic beads have not accumulated in the collection channel, the process returns to 2008 and the source fluid continues to flow into the source channel.

Generally, the method comprises first passing a source fluid through a source fluid channel within a microfluidic device, where the source fluid contains magnetic particles attached to target components; placing a collection fluid in a collection fluid channel within the microfluidic device, such that the collection fluid channel is in communication with the source fluid channel via one or more discrete transfer channels; and applying a magnetic field gradient to the source fluid, such that the magnetic field gradient causes the magnetic particles and the magnetic particle bound target components to migrate from the source fluid channel into the collection fluid channel via the at least one discrete transfer channel.

The affinity/binding molecule coated magnetic particles can be added into the source fluid prior to the source fluid being supplied to the source fluid channel. In some embodiments, semi-batch mixing processes are provided that allow longer bead-pathogen incubation periods while maintaining continuous source fluid, e.g., blood, flow. Such processes also enable integration into conventional continuous veno-venous hemafiltration units, which use hemaconcentrators, blood warmers and oxygenation technologies. In some further embodiments, additional safety features such as ultra-high-efficiency magnetic traps are also be added to the devices described herein to remove all remaining magnetic particles before the cleansed biological fluid is returned to the biological system, such as a septic patient.

After removal of the desired target component, the “cleansed” source fluid and/or the collection fluid containing the target components can be transferred for further processing, such as detection or analysis. In some embodiments of the invention, the cleansed fluid can be returned to the source. In the case of biological fluids, the cleansed biological fluid can be returned to the originating biological system, or to another subject or to a culture medium, biological scaffold, bioreactor, or the like. In some embodiments, it can be desirable to subject the cleansed biological fluid to post processing, for example, further treatment, filtering or a (blood) warming process prior to being returned to the originating biological system. Further, if desired, at least a portion of the “cleansed” source fluid can be recirculated back into the source fluid channel.

One can also collect at least a portion of the collection fluid and magnetic particles from the collection channel. The magnetic particles can be separated from the collection fluid prior to detecting whether any of the magnetic particles contain a target component. The separated magnetic particles can be analyzed to quantify the amount of target components attached to the magnetic particles.

The method can further comprise initiating flow for a selected amount of time, where the magnetic particles in the collection fluid are removed from the microfluidic device. The passing of the collection fluid can further comprise intermittently passing the collection fluid through the collection fluid channel at irregular or periodic intervals.

In one or more embodiments of this aspect, the source fluid is selected from one or more in a group comprising blood, cord blood, serum, plasma, urine, liquefied stool sample, cerebrospinal fluid, amniotic fluid, lymph, mucus, tears, tracheal aspirate, sputum, saline, a buffer, a physiological salt solution or a cell culture medium.

In one or more embodiments of this aspect, the collection fluid is isotonic saline.

In one or more embodiments of this aspect, the target components are selected from the group consisting of a pathogen, a stem cell, a cancer cell, a fetal cell, a blood cell or an immune cell, a cytokine, a hormone, an antibody, a blood protein, or a molecular or chemical toxin.

The various aspect disclosed herein can be described by one or more of the following numbered paragraphs:
1. A microfluidic device comprising:
   (i) a central body comprising
      a. on a first outer surface, a source channel connected between a source inlet and a source outlet;
      b. on a second outer surface, a collection channel connected between a collection inlet and a collection outlet; and
      c. at least one transfer channel connecting the source channel and the collection channel;
   (ii) a first laminating layer in contact with the first outer surface of the central body, wherein the source inlet is in communication with a source inlet port on an outer surface of the first laminating layer and the source outlet is in communication with a source outlet port on the outer surface of the first laminating layer, and the first laminating layer and the first outer surface of the central body defining the source channel;
   (iii) a second laminating layer in contact with the second outer surface of the central body, wherein the collection inlet is in communication with a collection inlet port on an outer surface of the second laminating layer and the collection outlet is in communication with a collection outlet port on the outer surface of the second laminating layer, and the second laminating layer and second outer surface of the central body defining the collection channel; and
   (iv) one or more magnetic field gradient sources disposed adjacent to the collection channel and configured to apply a magnetic field gradient to a fluid flowing in the source channel and to cause target components in the source channel to migrate into the at least one transfer channel or the collection channel.
2. The microfluidic device according to paragraph 1, further comprising:
   (i) a fluid source connected to the source inlet port for delivering a source fluid to the source channel, the source fluid including target components to be removed from the source fluid; and
   (ii) a collection fluid source connected to the collection inlet port for delivering a collection fluid to the collection channel to fill the collection channel and the at least one transfer channel.
3. The microfluidic device according to any of paragraphs 1-2, wherein at least one fluid contacting surface, of the source channel, the collection channel, or the at least one transfer channel is an anti-coagulant surface.
4. The microfluidic device according to paragraph 3, wherein the fluid contacting surface is a slippery liquid-infused porous surface (SLIPS).
5. The microfluidic device according to paragraph 3 or 4, wherein the fluid contacting surface is coated with an anti-coagulant agent.
6. The microfluidic device according to any of paragraphs 1-5, wherein the first laminating layer has a thickness of about 0.01 mm to about 10 mm.
7. The microfluidic device according to paragraph 6, wherein the first laminating layer has a thickness of about 0.07 mm about 0.1 mm.
8. The microfluidic device according to any of paragraphs 1-7, wherein the second laminating layer has a thickness of about 0.01 mm to about 10 mm.
9. The microfluidic device according to paragraph 6, wherein the second laminating layer has a thickness of about 0.07 mm to about 0.1 mm.
10. The microfluidic device according to any of paragraphs 1-9, further comprising an inline mixer device connected to the source inlet and adapted to deliver a plurality of magnetic particles to the source fluid.
11. The microfluidic device according to any of paragraphs 1-10, further comprising an inline bubble-trapping device connected directly or indirectly to:
   a. the source inlet; or
   b. the source outlet.
12. The microfluidic device according to any of paragraphs 1-11, wherein the distance between the source channel and the collection channel is from about 10 μm to about 10 mm.
13. The microfluidic device according to paragraph 12, wherein the distance between the source channel and the collection channel is about 500 μm.
14. The microfluidic device according to any of paragraphs 1-13, wherein the source channel and the collection channel independently have a length of about 1 mm to about 10 cm, a width of about 0.1 mm to about 100 mm and a depth of about 0.1 mm to about 20 mm.
15. The microfluidic device according to any of paragraphs 1-14, wherein the source channel and the collection channel have substantially similar dimensions.
16. The microfluidic device according to any of paragraphs 1-15, wherein the source channel has a length of about 25 mm, a width of about 2 mm, and depth of about 0.6 mm.
17. The microfluidic device according to any of paragraphs 1-16, wherein the collection channel has a length of about 25 mm, a width of about 2 mm, and depth of about 0.6 mm.
18. The microfluidic device according to any of paragraphs 1-17, wherein the at least one transfer channel has cross-sectional dimensions of about 200 μm×10 mm to about 1 mm×100 mm.
19. The microfluidic device according to paragraph 18, wherein the at least one transfer has cross-sectional dimensions of about 400 μm×2 mm.
20. The microfluidic device according to any of paragraphs 1-19, wherein spacing between the transfer channels is about 10 μm to about 5 mm.
21. The microfluidic device according to paragraph 20, wherein spacing between the transfer channels is about 3 mm.
22. The microfluidic device according to any of paragraphs 1-21, wherein the device has a length of about 2 cm to about 100 cm, a width of about 2 cm to about 100 cm, and a width of about 2 cm to about 100 cm.
23. The microfluidic device according to any of paragraphs 1-22, wherein the device has a length of about 128 mm, a width of about 57 mm, and a depth of about 2 mm.
24. The microfluidic device according to any of paragraphs 1-23, wherein the device has a length of about 128 mm, a width of about 57 mm, and a depth of about 2 mm; wherein the source channel has a length of about 25 mm, a width of about 2 mm, and depth of about 0.6 mm; wherein the collection channel has a length of about 25 mm, a width of about 2 mm, and depth of about 0.6 mm; wherein the at least one transfer has cross-sectional dimensions of about 400 μm×2 mm; and wherein spacing between the transfer channels is about 3 mm.
25. The microfluidic device according to any of paragraphs 1-24, wherein at least one of the transfer channels is oriented at an angle of less than 90 degrees to the source channel.
26. The microfluidic device according to any of paragraphs 1-25, wherein the central body, the first laminating layer, or the second laminating layer are fabricated from a biocompatible material.
27. The microfluidic device according to any of paragraphs 1-26, wherein the central body, the first laminating layer, or the second laminating layer are fabricated from an FDA-approved blood-compatible material.
28. The microfluidic device according to any of paragraphs 1-27, wherein the central body, the first laminating layer, or the second laminating layer are fabricated from a material selected from the group consisting of aluminum, polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidine fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), stainless steels, titanium, platinum, alloys, ceramics and glasses non-magnetic metals, and any combination thereof
29. The microfluidic device according to any of paragraphs 1-28, wherein the magnetic field gradient is sufficient to cause the target components in the source channel to migrate into the at least one collection channel.
30. The microfluidic device according to any of paragraphs 1-29, wherein the source fluid is a biological fluid selected from the group consisting of blood, plasma, serum, lactation products, milk, amniotic fluids, peritoneal fluid, sputum, saliva, urine, semen, cerebrospinal fluid, bronchial aspirate, perspiration, mucus, liquefied stool sample, synovial fluid, lymphatic fluid, tears, tracheal aspirate, and any mixtures thereof.
31. The microfluidic device according to any of paragraphs 1-30, wherein the source fluid is a non-biological fluid selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof
32. The microfluidic device according to any of paragraphs 1-31, wherein the collection fluid is selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof
33. The microfluidic device according to paragraph 32, wherein the collection fluid is isotonic saline, a biological fluid, a biocompatible fluid or a biological fluid substitute.
34. The microfluidic device according to any of paragraphs 1-33, further comprising an inline diagnostic device connected to the collection outlet adapted to analyze the target components in the collection fluid.
35. The microfluidic device according to paragraph 34, wherein the inline diagnostic device includes a magnetic field gradient source, adjacent to a collection chamber, adapted to cause the target components in the collection fluid to collect in the collection chamber.
36. The microfluidic device according to any of paragraphs 1-35, wherein
   a. the source fluid flows at a rate of 1 mL/hr to 2000 mL/hr through the source channel; and
   b. the collection fluid flows at a rate of 1 mL/hr to 2000 mL/hr through the collection channel.
37. The microfluidic device according to any of paragraphs 1-36, wherein the target component is attracted or repelled by a magnetic field gradient.
38. The microfluidic device according to any of paragraphs 1-37, wherein the target component is bound to a particle that is attracted or repelled by a magnetic field gradient.
39. The microfluidic device according to any of paragraphs 1-38, wherein the target component is bound to a binding/affinity molecule that is bound to a particle that is attracted or repelled by a magnetic field gradient.
40. The microfluidic device according to paragraph 39, wherein the binding/affinity molecule is selected from the group consisting of antibodies, antigens, proteins, peptides, nucleic acids, receptor molecules, ligands for receptors, lectins, carbohydrates, lipids, one member of an affinity binding pair, and any combination thereof
41. The microfluidic device according to paragraph 39 or 40, wherein the binding/affinity molecule is selected from the group consisting of MBL (mannose binding lectin), FcMBL (IgG Fc fused to mannose binding lectin), AKT-FcMBL (IgG Fc fused to mannose binding lectin with the N-terminal amino acid tripeptide of sequence AKT (alanine, lysine, threonine)), and any combination thereof.
42. The microfluidic device according to any of paragraphs 39-41, wherein the binding/affinity molecule comprises an amino acid sequence selected from SEQ ID NO. 1, SEQ ID NO. 2, SEQ ID NO. 3, SEQ ID NO. 4, SEQ ID NO. 5, SEQ ID NO. 6, SEQ ID NO. 6, SEQ ID NO. 7, SEQ ID NO. 8, and any combination thereof.
43. The microfluidic device according to any of paragraphs 38-42, wherein the particle is paramagnetic.
44. The microfluidic device according to any of paragraphs 38-43, wherein the particle is of size in range from 0.1 nm to 500 μm.
45. The microfluidic device according to any of paragraphs 38-44, wherein the particle is spherical, rod, elliptical, cylindrical, or disc shaped.
46. The microfluidic device according to any of paragraphs 1-45, wherein the target component is a bioparticle/pathogen selected from the group consisting of living or dead cells (prokaryotic or eukaryotic), viruses, bacteria, fungi, yeast, protozoan, microbes, parasites, and the like.
47. The microfluidic device according to paragraph 46, wherein the target component is:
   a. fungi or yeast selected from the group consisting Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii (or Pneumocystis carinii), Stachybotrys chartarum, and any combination thereof;
   b. bacteria selected from the group consisting of anthrax, campylobacter, cholera, diphtheria, enterotoxigenic E. coli, giardia, gonococcus, Helicobacter pylori, Hemophilus influenza B, Hemophilus influenza non-typable, meningococcus, pertussis, pneumococcus, salmonella, shigella, Streptococcus B, group A Streptococcus, tetanus, Vibrio cholerae, Yersinia, Staphylococcus, Pseudomonas species, Clostridia species, Myocobacterium tuberculosis, Mycobacterium leprae, Listeria monocytogenes, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Treponema pallidum, Haemophilus influenzae, Treptonema pallidum, Klebsiella pneumoniae, Pseudomonas aeruginosa, Cryptosporidium parvum, Streptococcus pneumoniae, Bordetella pertussis, Neisseria meningitides, and any combination thereof;
   c. parasite selected from the group consisting of Entamoeba histolytica; Plasmodium species, Leishmania species, Toxoplasmosis, Helminths, and any combination thereof;
   d. virus selected from the group consisting of HIV-1, HIV-2, hepatitis viruses (including hepatitis B and C), Ebola virus, West Nile virus, and herpes virus such as HSV-2, adenovirus, dengue serotypes 1 to 4, ebola, enterovirus, herpes simplex virus 1 or 2, influenza, Japanese equine encephalitis, Norwalk, papilloma virus, parvovirus B19, rubella, rubeola, vaccinia, varicella, Cytomegalovirus, Epstein-Barr virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, poliovirus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Human T-cell Leukemia virus type-1, Hantavirus, Rubella virus, Simian Immunodeficiency viruses, and any combination thereof; or
   e. any combination of (a)-(d).
48. The microfluidic device according to paragraph 46, wherein the target component is a cell selected from the group consisting of stem cells, cancer cells, progenitor cells, immune cells, blood cells, fetal cells, and the like.
49. The microfluidic device according to any of paragraphs 1-48, wherein the target component is selected from the group consisting of hormones, cytokines, proteins, peptides, prions, lectins, oligonucleotides, molecular or chemical toxins, and any combination thereof.
50. A system comprising:
   (i) a microfluidic device according to any of paragraphs 1-49;
   (ii) a fluid source connected to the source channel and delivering a source fluid to the source channel, the source fluid including target components to be removed from the source fluid;
   (iii) a source pump, connected to the source channel, and adapted to pump the source fluid into the source channel;
   (iv) a source mixer, connected to the source channel and the fluid source, and adapted to mix the source fluid with magnetic particles;
   (v) a collection fluid source connected to the collection inlet and adapted to deliver a collection fluid to the first collection channel and to draw the target components from the at least one transfer channel into the collection channel and flush the target components from the collection channel;
   (vi) a collection pump, connected to the collection inlet and the collection fluid source, and adapted to pump the collection fluid into the collection channel; and
   (vii) a controller, having a processor and associated memory, and being coupled to
      a. the source pump to control the flow of source fluid through the source channel, and
      b. the collection pump to control the flow of the collection fluid through the collection channel.
51. The system according to paragraph 50, further comprising an inline diagnostic device, connected to the collection outlet and adapted to analyze the target component in the collection fluid.
52. The system according to paragraph 51, wherein the inline diagnostic device includes a magnetic field gradient source, adjacent to a collection chamber, adapted to cause the target components in the first collection fluid to collect in the collection chamber.
53. The system according to any of paragraphs 51-52, wherein the inline diagnostic device uses one or more of dyes, antibodies, non-labeled optical techniques, or solid-state detection techniques to analyze the target components.
54. The system according to any of paragraphs 50-53, wherein the magnetic field gradient is sufficient to cause the target components in the source channel to migrate into the collection channel.
55. A method of cleansing a source fluid, the method comprising:
   i. providing a microfluidic device according to any of paragraphs 1-50;
   ii. causing a source fluid to flow thru the source channel, wherein the source fluid includes a target component to be removed/separated from the source fluid;
   iii. providing a collection fluid in the collection channel;
   iv. applying a magnetic field gradient to the source fluid in the source channel, whereby the target components migrate into one of the at least one transfer channel.
56. The method according to paragraph 55, further comprising causing the collection fluid to flow thru the collection channel, wherein the target components in the collection fluid are removed from the collection channel.
57. The method according to paragraph 55 or 56, further comprising causing the collection fluid to flow continuously thru the collection channel, wherein the target components in the collection fluid are removed from the collection channel.
58. The method according to any of paragraphs 56 or 57, further comprising causing the collection fluid to flow at periodic intervals thru the collection channel, wherein the target components in the collection fluid are removed from the collection channel.
59. The method according to any of paragraphs 55-58, wherein the source fluid is a biological fluid selected from the group consisting of blood, plasma, serum, lactation products, milk, amniotic fluids, peritoneal fluids sputum, saliva, urine, semen, cerebrospinal fluid, bronchial aspirate, perspiration, mucus, liquefied stool sample, synovial fluid, lymphatic fluid, tears, tracheal aspirate, and any mixtures thereof.
60. The method according to any of paragraphs 55-58, wherein the source fluid is a non-biological fluid selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof
61. The method according to any of paragraphs 55-60, wherein the collection fluid is selected from the group consisting of water, organic solvents, saline solutions, sugar solutions, carbohydrate solutions, lipid solutions, nucleic acid solutions, hydrocarbons, acids, gasoline, petroleum, liquefied foods, gases, and any mixtures thereof
62. The method according to any of paragraphs 55-61, wherein the collection fluid is isotonic saline, a biological fluid, a biocompatible fluid or a biological fluid substitute.
63. The method according to any of paragraphs 55-62, wherein the target component is attracted or repelled by a magnetic field gradient.
64. The method according to any of paragraphs 55-63, wherein the target component is bound to a particle that is attracted or repelled by a magnetic field gradient.
65. The method according to any of paragraphs 55-64, wherein the target component is bound to a binding/affinity molecule that is bound to a particle that is attracted or repelled by a magnetic field gradient.
66. The method according to paragraph 65, wherein the binding/affinity molecule is selected from the group consisting of antibodies, antigens, proteins, peptides, nucleic acids, receptor molecules, ligands for receptors, lectins, carbohydrates, lipids, one member of an affinity binding pair, and any combination thereof
67. The method according to paragraph 65 or 66, wherein the binding/affinity molecule is selected from the group consisting of MBL (mannose binding lectin), FcMBL (IgG Fc fused to mannose binding lectin), AKT-FcMBL (IgG Fc fused to mannose binding lectin with the N-terminal amino acid tripeptide of sequence AKT (alanine, lysine, threonine)), and any combination thereof
68. The method according to any of paragraphs 65-67, wherein the binding/affinity molecule comprises an amino acid sequence selected from SEQ ID NO. 1, SEQ ID NO. 2, SEQ ID NO. 3, SEQ ID NO. 4, SEQ ID NO. 5, SEQ ID NO. 6, SEQ ID NO. 6, SEQ ID NO. 7, SEQ ID NO. 8, and any combination thereof.
69. The method according to any of paragraphs 64-68, wherein the particle is paramagnetic.
70. The method of any of paragraphs 64-69, wherein the particle is of size in range from 0.1 nm to 1 mm.
71. The method according to any of paragraphs 64-70, wherein the particle is spherical, rod, elliptical, cylindrical, or disc shaped.
72. The method according to any of paragraphs 55-71, wherein the target component is a bioparticle/pathogen selected from the group consisting of living or dead cells (prokaryotic or eukaryotic), viruses, bacteria, fungi, yeast, protozoan, microbes, parasites, and the like.
73. The method according to paragraph 72, wherein the target component is:
   a. fungi or yeast selected from the group consisting Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii (or Pneumocystis carinii), Stachybotrys chartarum, and any combination thereof;
   b. bacteria selected from the group consisting of anthrax, campylobacter, cholera, diphtheria, enterotoxigenic E. coli, giardia, gonococcus, Helicobacter pylori, Hemophilus influenza B, Hemophilus influenza non-typable, meningococcus, pertussis, pneumococcus, salmonella, shigella, Streptococcus B, group A Streptococcus, tetanus, Vibrio cholerae, Yersinia, Staphylococcus, Pseudomonas species, Clostridia species, Myocobacterium tuberculosis, Mycobacterium leprae, Listeria monocytogenes, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Treponema pallidum, Haemophilus influenzae, Treptonema pallidum, Klebsiella pneumoniae, Pseudomonas aeruginosa, Cryptosporidium parvum, Streptococcus pneumoniae, Bordetella pertussis, Neisseria meningitides, and any combination thereof;
   c. parasite selected from the group consisting of Entamoeba histolytica; Plasmodium species, Leishmania species, Toxoplasmosis, Helminths, and any combination thereof;
   d. virus selected from the group consisting of HIV-1, HIV-2, hepatitis viruses (including hepatitis B and C), Ebola virus, West Nile virus, and herpes virus such as HSV-2, adenovirus, dengue serotypes 1 to 4, ebola, enterovirus, herpes simplex virus 1 or 2, influenza, Japanese equine encephalitis, Norwalk, papilloma virus, parvovirus B19, rubella, rubeola, vaccinia, varicella, Cytomegalovirus, Epstein-Barr virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, poliovirus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Human T-cell Leukemia virus type-1, Hantavirus, Rubella virus, Simian Immunodeficiency viruses, and any combination thereof; or
   e. any combination of (a)-(d).
74. The method according to paragraph 72, wherein the target component is a cell selected from the group consisting of stem cells, cancer cells, progenitor cells, immune cells, blood cells, fetal cells, and the like.
75. The method according to any of paragraphs 55-71, wherein the target component is selected from the group consisting of hormones, cytokines, proteins, peptides, prions, lectins, oligonucleotides, molecular or chemical toxins, exosomes, and any combination thereof
76. The method according to any of paragraphs 64-75, further comprising adding the particle into the source fluid before initiating flow of the source fluid thru the source channel.
77. The method according to any of paragraphs 64-75, further comprising adding the particles into the source fluid after initiating flow of the source fluid thru the source channel.
78. The method according to any of paragraphs 55-77, further comprising collecting at least a portion of the collection fluid from the collection channel.
79. The method according to any of paragraphs 55-78, further comprising recycling a portion of the source fluid for a second pass thru the source channel for further separation of target components.
80. The method according to any of paragraphs 55-79, wherein at least 10% of the target components are removed from the source fluid.
81. The method according to any of paragraphs 55-80, wherein the source fluid flows at rate of 1 mL/hr to 2000 mL/hr thru the source channel.
82. The method according to any of paragraphs 55-81, wherein the collection fluid flows at a rate of 1 mL/hr to 2000 mL/hr thru the collection channel.
83. The method according to any of paragraphs 55-82, wherein the flow rate thru the collection channel is intermittent.
84. The method according to paragraph 83, wherein the collection fluid flow is off until a predefined volume of source fluid has passed through the source channel and then the collection fluid flow is turned on for a predefined time at a predefined flow rate.
85. The method according to paragraph 84, wherein the flow through the source channel is stopped while the collection fluid flows through the collection channel.
86. The method according to any of paragraphs 55-85, further comprising collecting the collection fluid containing the target component in a collection fluid collector, removing at least one target component from the collection fluid collector and analyzing the removed target component using one or more of the processes from the group including immuno-staining, culturing, PCR, mass spectrometry and antibiotic sensitivity testing.
87. The method according to any of paragraphs 55-86, further comprising providing an inline diagnostic device connected to the collection outlet adapted to analyze the target components in the collection fluid.
88. The method according to paragraph 87, wherein the inline diagnostic device includes a magnetic field gradient source adjacent to a collection chamber adapted to cause the target components in the collection fluid to collect in the collection chamber.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. The mammal can be a human, non-human primate, mouse, rat, rabbit, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with sepsis, inflammatory diseases, or infections.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### High-Flow Microfluidics

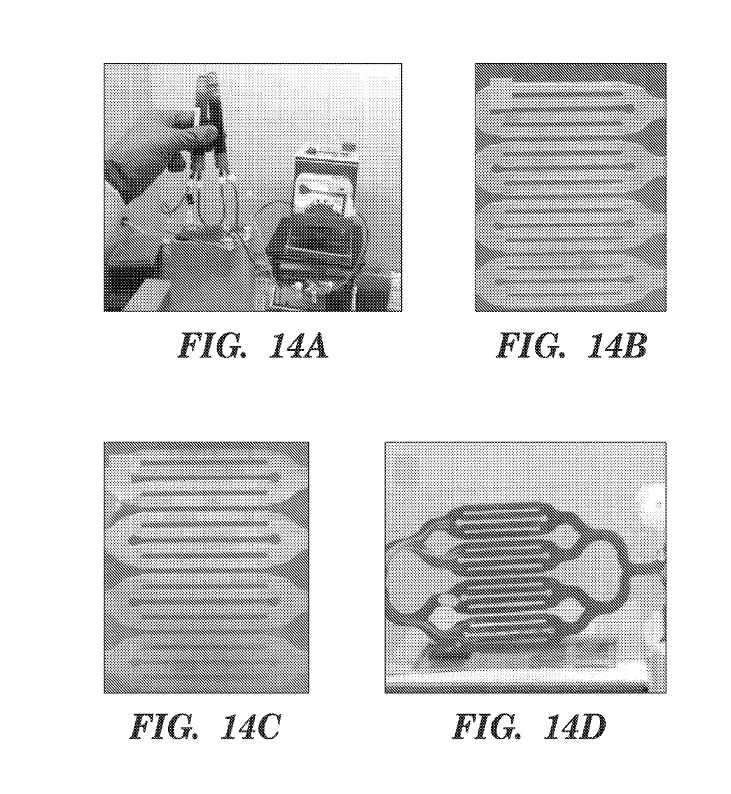

Microfluidic devices were fabricated from polysulfone, an FDA-approved blood compatible material. The devices were laminated by an optically clear film covered with adhesive on one side. Previously, the inventors had examined the capability of the devices at high flow rates at up to 360 mL/h; however, blood was infused for a short period of time. Accordingly, the inventors circulated heparinized human whole blood collected from healthy human donors at flow rates of 100 and 200 mL/h for 2 hours (FIG. 14) After circulating blood through the devices, blood remaining in the channels was washed by PBS buffer. No blood clots were formed by shear stress in the devices. However, when circulating non-heparinized human whole blood through the devices for 2 hours, the inventors found several large blood clots that adhered to the channel surface. Applying anti-coagulant surfaces, e.g., SLIPS, to the devices can solve this issue.

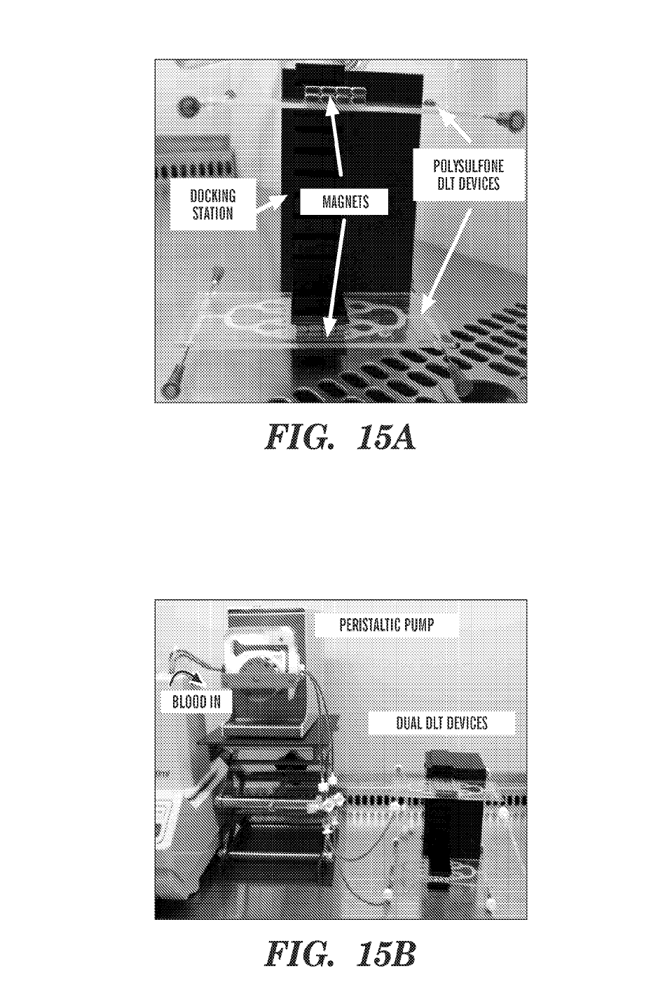

Moreover, inventors have connected two polysulfone based microfluidic devices in parallel to dramatically increase throughput (836 mL/h in total, 418 mL/h in each device). The inventors successfully demonstrated that blood (CPDA-1 added) bifurcated into the two microfluidic devices linked in parallel, where difference in flows between two devices was determined less than 5% at a flow rate of 836 mL/h in total. (FIGS. 15A and 15B). This shows that one can integrate multiple microfluidic devices in parallel so that microfluidic devices of the invention can be used for processing and cleansing a large blood volume of septic patients.

The microfluidic devices for obtaining anticoagulant SLIP surface are treated by a succession of physicochemical processes which operate in extreme conditions requiring tolerance to high temperature and mechanical stress. Thus, the inventors also made the microfluidic device using aluminum (FIG. 6). Aluminum provides an easy fabrication and capability to tolerate many surface modification processes, including chemical vapor deposition, chemical cleansing processes, polymer deposition at high temperatures. The aluminum devices were also laminated by an optically clear film and then the inventors infused human blood (1 unit of CPDA-1 added) through the device at 418 mL/h for 5 min. This data showed that the aluminum DLT device did not cause any blood clot formation for a short period of time even at high flow rate (418 mL/h in a single device).

### Example 2

### Sepsis Animal Model

Inventors have improved upon the previous microfluidic device designs to enhance isolation efficiency of 1 μm MBL conjugated magnetic bead bound pathogens. To leverage high magnetic flux density gradients across the device to pull the magnetic bead bound pathogens, the inventors replaced the top and bottom polysulfone layers with a thin polymer film coated with adhesive on a side, which reduces a distance between a stationary magnet and the blood channel on the bottom where the magnetic beads bound pathogens flow through. Because the magnetic flux density gradient decreases dramatically as the distance from a magnet increases, this improved fabrication method allows us to utilize the extremely strong magnetic force nearby a magnet surface. Moreover, computational simulation studies to estimate magnetic fields around magnets more accurately revealed that we can improve the magnetic forces by modifying the geometry of magnets. As shown in FIGS. 5A-5C, the magnetic flux density gradients in the new design were estimated to be around at most ˜10³ times larger than the previous magnet setup. This theoretical estimation was proved by comparing the isolation efficiency obtained from those two experimental setups; a single magnet (4″×1″×⅛″, NdFeB N42) and assembled magnets (2″×¼″×⅛″, NdFeB N42, magnetized through thickness).

Moreover, the inventors changed the shape of transfers channels in the microfluidic device through which the magnetic bead bound pathogens are pulled by magnetic forces and dragged from the source channel into the collection channel. In the previous design, the magnetic bead bound pathogens were most likely stuck on the channel wall in between arrays of circular through-holes, which can prevent one from retrieving the isolated pathogens. Thus, the inventors modified the shape of transfer channels. The inventors made transfer channels or slits of cross-section 2 mm×400 μm (29 slits in each channel, 16 branched-channels in the device) in the middle of the channels to ensure that all magnetic beads and bead bound pathogens can be pulled into the saline channel through the slits and no bead-bound pathogens can be stuck on the wall of the DLT device. This new feature also enabled that the pathogens magnetically isolated can be retrieved after cleansing blood.

The inventors quantified the number of pathogens isolated in the DLT device by collecting magnetic bead-bound pathogens from the device and then plating them on the potato dextrose plates. The results revealed that one can collect the isolated pathogens from the DLT devices. In contrast, the previous devices with circular transfer channels was not capable of retrieving the isolated pathogens from the collection channels which is most likely attributed to the bead-bound pathogens stuck on the wall of the lower blood channel network in the device. This improved design with slits can enable one to carry out quantitative and qualitative analysis of the pathogens captured from blood of septic patients, which further offer clinicians additional information to treat the septic patients with more appropriate antibiotics that might avoid side effects.

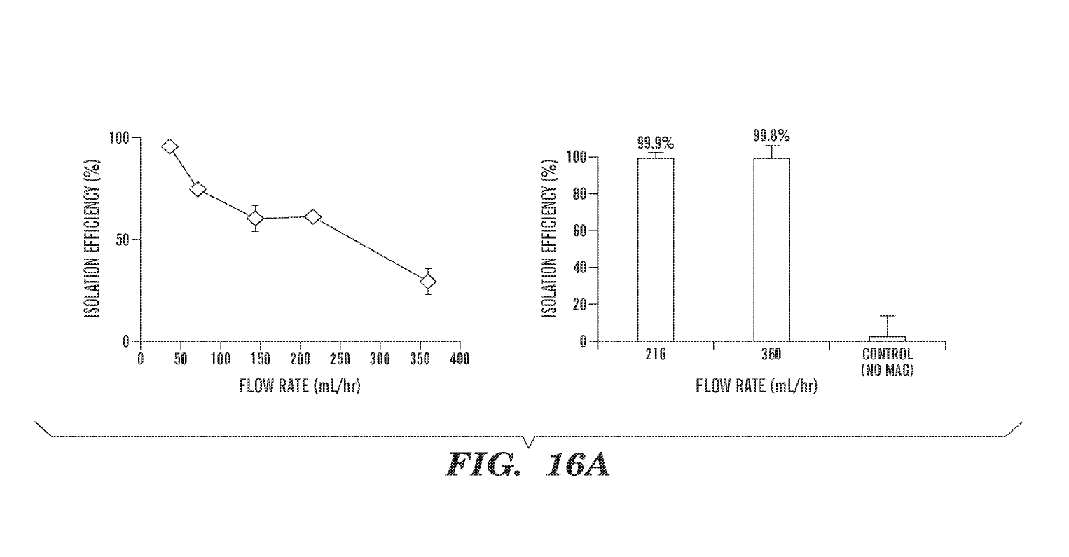

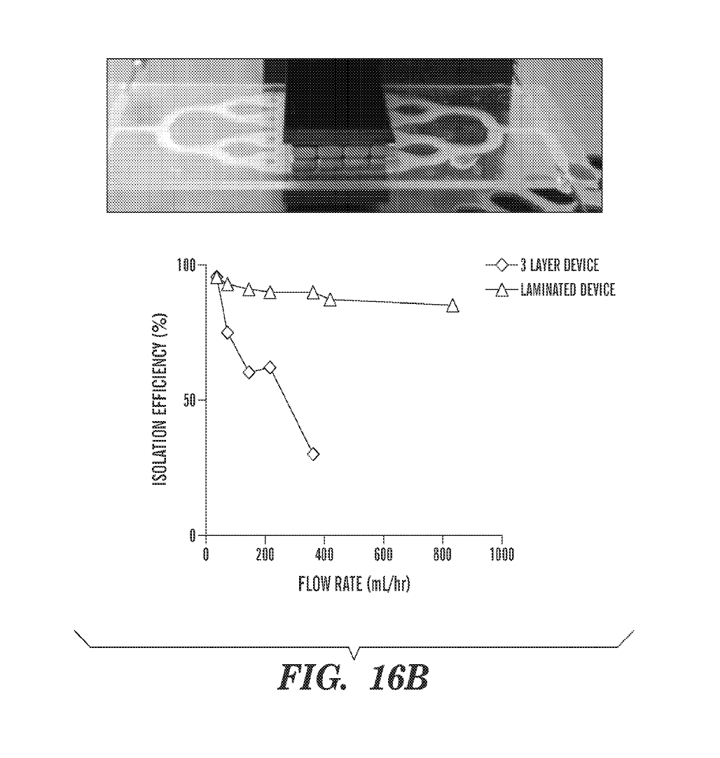

Combining these improved designs all together led to significantly improved isolation capability and increased throughput as shown in FIG. 16. Inventors quantified the isolation efficiency of the new design of the device. C. albicans that were bound to each 1 μm akt Fc MBL bead and 1 μm wild type MBL beads were spiked into human blood (CPDA-1) and removed from blood using the our improved DLT devices with efficiencies of above 90% even at 418 mL/h. As discussed in Example 1, 1 two devices linked in parallel produced comparable result (85% of isolation efficiency) even at a flow rate of 836 mL/h, where the inventors spiked 1 μm WT-MBL magnetic bead bound C. albicans into human blood (CPDA-1). The two DLT devices that ran in parallel produced similar isolation results (84.9% from the top DLT device and 85.6% from the DLT device on the bottom in FIG. 15), which cross-checks that blood was equally distributed into each DLT device. Moreover, this improved design utilizing enhanced magnetic forces can further permit efficiency isolation of bacteria using magnetic nanoparticles (114 nm in diameter) to capture them more efficiently. As a control experiment, the inventors flowed blood containing 1 μm magnetic bead bound C. albicans through the DLT device without the applied magnetic field, and no pathogen separation was observed.

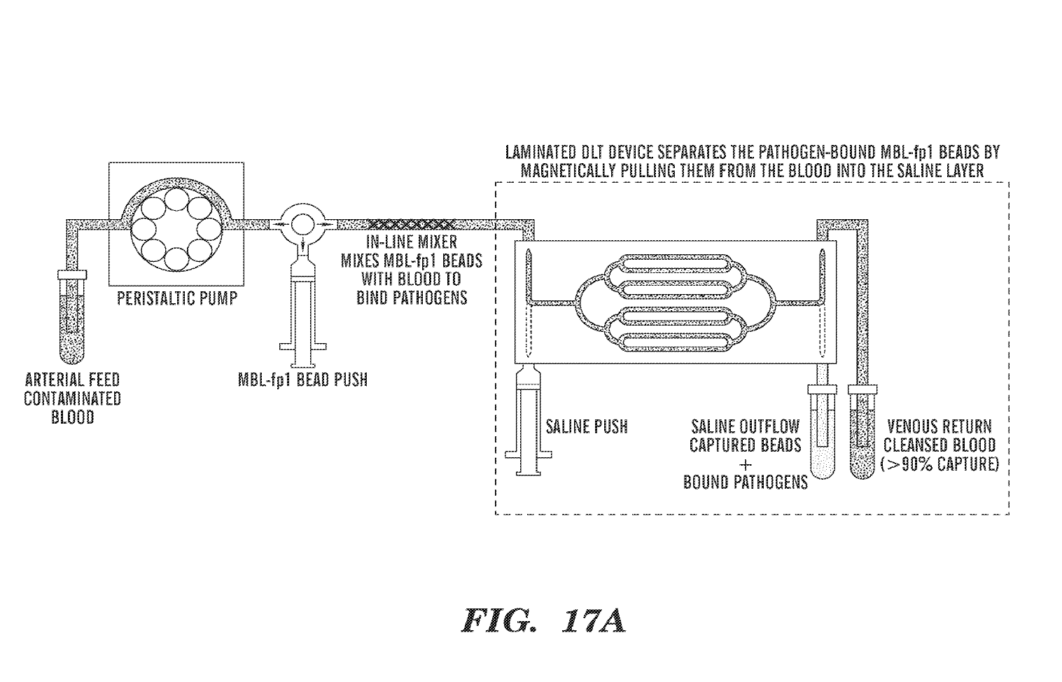

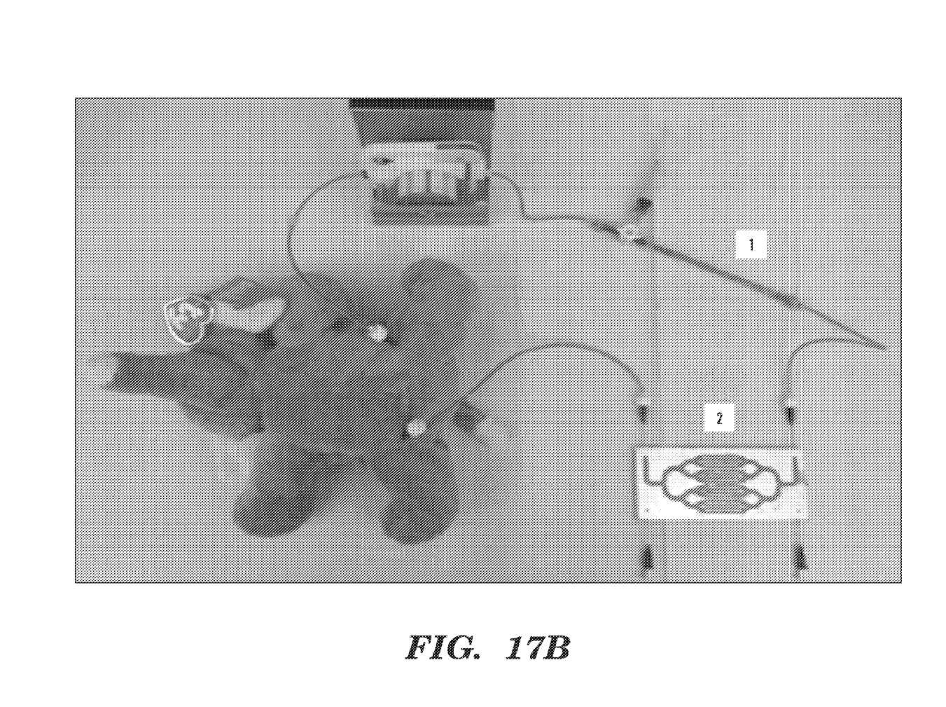

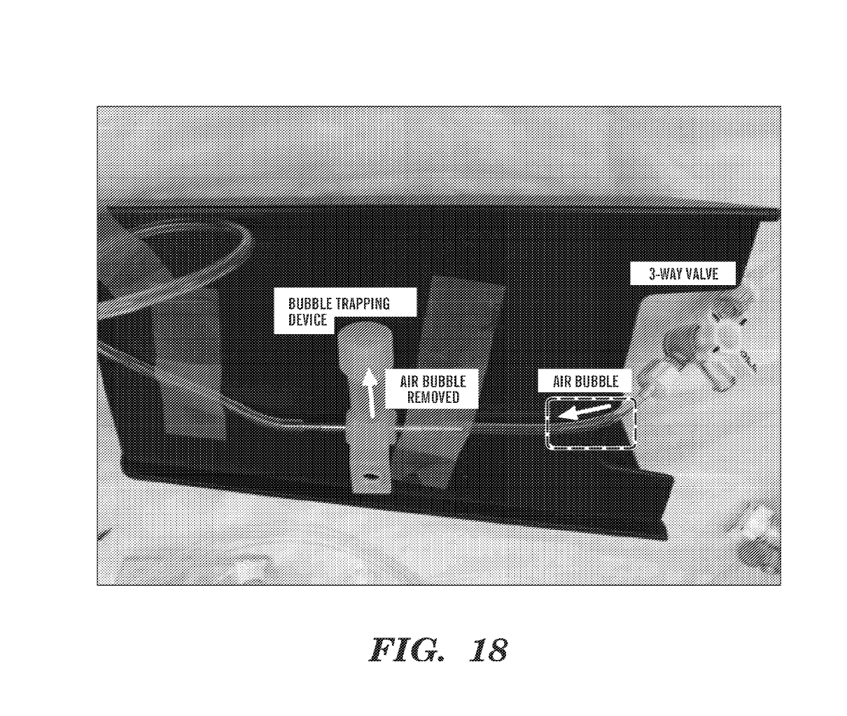

In addition, the inventors also integrated an in-line mixer into the DLT tubing to determine pathogen removal efficiency from blood that contains free pathogens, which mimics more realistic experimental conditions of cleansing septic blood (FIG. 17). The disposable in-line mixer that has been developed for mixing high viscous solution (OMEGA Engineering Inc., CT) consists of a series of mixing elements which have spiral baffles in a polymer tubing. The magnetic beads (1 μm akt Fc MBL, 3.5×10⁸ beads/mL) were introduced into the tubing at a flow rate of 7.1 μL/min where blood containing the spiked C. albicans flows through and then, blood and magnetic beads were mixed together in the in-line mixer placed in between the peristaltic pump and the DLT device. Assuming a flow rate (10 mL/h) of the DLT system in this condition, based on a previous study describing the blood flow rate in a femoral vein of a male Wistar rat (18 mL/h), and operating the DLT system on the rat sepsis model can further reveal an optimal flow rate at which the extracorporeal DLT system can circulate blood. With the given conditions (10 mL/h, 50 cm-long tubing), the blood sample (CPDA-1, 5 mM CaCl₂, spiked C. albicans) was mixed with the beads for ˜5 min, flowing through the DLT system and then, ˜88% of the spiked C. albicans were cleared from blood.

Finally, as described in Example 1, the inventors also made the DLT devices from aluminum to explore more options to build SLIPS surface on the DLT device channel networks. The aluminum DLT device has the same design parameters as the polysulfone DLT device. The inventors confirmed that the aluminum DLT device can isolate 1 μm magnetic bead bound C. albicans from blood with comparable isolation efficiency (−90%) at 418 mL/h.

### Example 3

### Rat Sepsis Model

The inventors modified the microfluidic device and the tubing setup to adjust the microfluidic system to the rat sepsis model. Small blood volume in rats enabled a reduction in the volume of the device and the tubing to prime with crystalloid solution to minimize dilution effect of blood in rats. The improved design of device has 1.2 mL of the blood channel network and 1 mL of the tubing whereas the previous device enabled 2.5 mL to prime the blood channel network. Moreover, because air bubbles in blood stream can cause lethal air embolism in in vivo models, the inventors also integrated a bubble trapping device (#25014, www.restek.com) with the DLT system (FIG. 18) to completely eliminate air bubbles in the microfluidic system. The air bubbles incidentally generated in the tubing can be completely removed. If an excessive amount of air bubbles comes in through the tubing, one can remove those bubbles through the 3-way valve prior to the bubble trapping device.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

To the extent not already indicated, it will be understood by those of ordinary skill in the art that any one of the various embodiments herein described and illustrated can be further modified to incorporate features shown in any of the other embodiments disclosed herein.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A microfluidic device comprising:
(i) a central body comprising
a. on a first outer surface, a source channel connected between a source inlet and a source outlet;
b. on a second outer surface, a collection channel connected between a collection inlet and a collection outlet; and
c. at least one transfer channel connecting the source channel and the collection channel;
(ii) a first laminating layer in contact with the first outer surface of the central body, wherein the source inlet is in communication with a source inlet port on an outer surface of the first laminating layer and the source outlet is in communication with a source outlet port on the outer surface of the first laminating layer, and the first laminating layer and the first outer surface of the central body defining the source channel;
(iii) a second laminating layer in contact with the second outer surface of the central body, wherein the collection inlet is in communication with a collection inlet port on an outer surface of the second laminating layer and the collection outlet is in communication with a collection outlet port on the outer surface of the second laminating layer, and the second laminating layer and second outer surface of the central body defining the collection channel; and
(iv) one or more magnetic field gradient sources disposed adjacent to the collection channel and configured to apply a magnetic field gradient to a fluid flowing in the source channel and to cause target components in the source channel to migrate into the at least one transfer channel or the collection channel.

2. (canceled)

3. The microfluidic device according to claim 1, wherein at least one fluid contacting surface, of the source channel, the collection channel, or the at least one transfer channel is an anti-coagulant surface.

4. The microfluidic device according to claim 3, wherein the fluid contacting surface is a slippery liquid-infused porous surface (SLIPS).

5-9. (canceled)

10. The microfluidic device according to claim 1, further comprising an inline mixer device connected to the source inlet and adapted to deliver a plurality of magnetic particles to the source fluid.

11. The microfluidic device according to claim 1, further comprising an inline bubble-trapping device connected directly or indirectly to:
a. the source inlet; or
b. the source outlet.

12-14. (canceled)

15. The microfluidic device according to claim 1, wherein the source channel and the collection channel have substantially similar dimensions.

16-24. (canceled)

25. The microfluidic device according to claim 1, wherein at least one of the transfer channels is oriented at an angle of less than 90 degrees to the source channel.

26. The microfluidic device according to claim 1, wherein the central body, the first laminating layer, or the second laminating layer are fabricated from a biocompatible material.

27-28. (canceled)

29. The microfluidic device according to claim 1, wherein the magnetic field gradient is sufficient to cause the target components in the source channel to migrate into the at least one collection channel.

30-33. (canceled)

34. The microfluidic device according to claim 1, further comprising an inline diagnostic device connected to the collection outlet adapted to analyze the target components in the collection fluid.

35. The microfluidic device according to claim 34, wherein the inline diagnostic device includes a magnetic field gradient source, adjacent to a collection chamber, adapted to cause the target components in the collection fluid to collect in the collection chamber.

36-37. (canceled)

38. The microfluidic device according to claim 1, wherein the target component is bound to a particle that is attracted or repelled by a magnetic field gradient.

39. The microfluidic device according to claim 1, wherein the target component is bound to a binding/affinity molecule that is bound to a particle that is attracted or repelled by a magnetic field gradient.

40-45. (canceled)

46. The microfluidic device according to claim 1, wherein the target component is a bioparticle/pathogen selected from the group consisting of living or dead cells (prokaryotic or eukaryotic), viruses, bacteria, fungi, yeast, protozoan, microbes, parasites, and the like.

47-49. (canceled)

50. A system comprising:
(i) a microfluidic device according to claim 1;
(ii) a fluid source connected to the source channel and delivering a source fluid to the source channel, the source fluid including target components to be removed from the source fluid;
(iii) a source pump, connected to the source channel, and adapted to pump the source fluid into the source channel;
(iv) a source mixer, connected to the source channel and the fluid source, and adapted to mix the source fluid with magnetic particles;
(v) a collection fluid source connected to the collection inlet and adapted to deliver a collection fluid to the first collection channel and to draw the target components from the at least one transfer channel into the collection channel and flush the target components from the collection channel;
(vi) a collection pump, connected to the collection inlet and the collection fluid source, and adapted to pump the collection fluid into the collection channel; and
(vii) a controller, having a processor and associated memory, and being coupled to
a. the source pump to control the flow of source fluid through the source channel, and
b. the collection pump to control the flow of the collection fluid through the collection channel.

51. The system according to claim 50, further comprising an inline diagnostic device, connected to the collection outlet and adapted to analyze the target component in the collection fluid.

52. The system according to claim 51, wherein the inline diagnostic device includes a magnetic field gradient source, adjacent to a collection chamber, adapted to cause the target components in the first collection fluid to collect in the collection chamber.

53-54. (canceled)

55. A method of cleansing a source fluid, the method comprising:
i. providing a microfluidic device according to claim 1;
ii. causing a source fluid to flow thru the source channel, wherein the source fluid includes a target component to be removed/separated from the source fluid;
iii. providing a collection fluid in the collection channel;
iv. applying a magnetic field gradient to the source fluid in the source channel, whereby the target components migrate into one of the at least one transfer channel.

56. The method according to claim 55, further comprising causing the collection fluid to flow thru the collection channel, wherein the target components in the collection fluid are removed from the collection channel.

57-88. (canceled)


SEQUENCE LISTING (8 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 380 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Description of Artificial Sequence Synthetic polypeptide
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 351 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI